(19) United States
(12) Patent Application Publication
(10) Pub. No.: US 2021/0008206 A1
(43) Pub. Date: Jan. 14, 2021

(54) ADENOSINE PATHWAY INHIBITORS FOR CANCER TREATMENT

(71) Applicant: Corvus Pharmaceuticals, Inc., Burlingame, CA (US)

(72) Inventors: Richard A. Miller, Portola Valley, CA (US); Ian Mccaffery, Oakland, CA (US); Andrew Hotson, Burlingame, CA (US)

(21) Appl. No.: 16/761,749

(22) PCT Filed: Nov. 6, 2018
(86) PCT No.: PCT/US2018/059480

§ 371 Date: May 5, 2020

Related U.S. Application Data
(60) Provisional application No. 62/582,246, filed on Nov. 6, 2017.

(51) Int. Cl. A61K 45/00; G01N 33/50; C07K 16/28; C07D 271/00; A61P 35/00

(52) U.S. Cl. CPC A61K 45/05; G01N 33/5094; A61P 35/00; C07D 271/00; C07K 16/2818

(57) ABSTRACT

Provided herein are, inter alia, methods for treating cancer in subjects expressing elevated levels of adenosine A2A receptors, and optionally further expressing elevated levels of CD73 and/or PD-L1, by administering adenosine pathway inhibitors.

### CROSS-REFERENCE TO RELATED APPLICATIONS

This application claims priority to U.S. Application No. 62/582,246 filed Nov. 6, 2017, the disclosure of which is incorporated by reference herein in its entirety.

### BACKGROUND

The goal of immunotherapy is to drive cytotoxic T-cell responses to eradicate cancer.

To prevent reaction to self-antigens, or overreaction, multiple inhibitory checkpoint signals exist including PD1/2, CTLA4, and adenosine. Extracellular adenosine, a purine nucleoside, is produced during acute, inflammatory processes by conversion from adenosine triphosphate (ATP) through ectonucleotidases CD73 and CD39 expressed on the cell surface of multiple tissue types. Adenosine is normally upregulated to protect a host from over-injury in response to such stimuli as infection or ischemia by binding its extracellular, G-protein coupled receptors on target cells and begin healing.

There is a need in the art for effective cancer treatments. The methods and compositions provided herein are directed to this, as well as other, important ends.

### BRIEF SUMMARY

Provided here are methods to treat cancer in patients by administering therapeutically effective amounts of adenosine pathway inhibitors when the patient has elevated levels of adenosine A2A receptors and, optionally, elevated levels of CD73 and/or elevated levels of PD-L1. Provided here are methods to treat cancer in patients by administering therapeutically effective amounts of adenosine pathway inhibitors when the patient has elevated levels of both adenosine A2A receptors and CD73.

The disclosure provides methods of treating cancer in a subject in need thereof by administering a therapeutically effective amount of an adenosine pathway inhibitor to the subject to treat the cancer; wherein the subject has an elevated level of adenosine A2A receptors when compared to a control. In embodiments, the adenosine pathway inhibitor is an adenosine A2A receptor antagonist, an anti-CD73 compound, an anti-CD39 compound, or a combination of two or more thereof. In embodiments, the adenosine pathway inhibitor is an adenosine A2A receptor antagonist. In embodiments, the adenosine A2A receptor antagonist is a compound of Formula (I), Formula (I), Formula (II), Formula (III), Formula (IIIA), Formula (IIIB), or a pharmaceutically acceptable salt thereof. In embodiments, the subject has been previously treated with PD-1 pathway inhibitor therapy, such as a PD-1 inhibitor and/or a PD-L1 inhibitor. In embodiments, the subject is an anti-PD-1 refractory subject or an anti-PD-1 resistant subject. In embodiments, the method of treating cancer is: (i) a method of increasing CD8-positive cells relative to the amount of regulatory T cells; (ii) a method of decreasing tumor volume; (iii) a method of enhancing anti-tumor immune memory; (iv) a method of treating a cancer tumor; or (v) two or more of the foregoing. In embodiments, the cancer is lung cancer, melanoma, breast cancer, colorectal cancer, bladder cancer, head and neck cancer, renal cell cancer, or prostate cancer. In embodiments, the subject is further administered a therapeutically effective amount of a chemotherapeutic agent, a therapeutically effective amount of an anti-CD73 antibody, or a combination thereof to treat the cancer.

The disclosure provides methods of treating cancer in a subject in need thereof by administering a therapeutically effective amount of an adenosine pathway inhibitor to the subject to treat the cancer; wherein the subject has: (i) an elevated level of adenosine A2A receptors when compared to a control; and (ii) an elevated level of CD73 when compared to a control. In embodiments, the adenosine pathway inhibitor is an adenosine A2A receptor antagonist, an anti-CD73 compound, an anti-CD39 compound, or a combination of two or more thereof. In embodiments, the adenosine pathway inhibitor is an adenosine A2A receptor antagonist. In embodiments, the adenosine A2A receptor antagonist is a compound of Formula (I), Formula (II), Formula (III), Formula (IIIA), Formula (IIIB), or a pharmaceutically acceptable salt thereof. In embodiments, the subject has been previously treated with PD-1 pathway inhibitor therapy, such as a PD-1 inhibitor and/or a PD-L1 inhibitor. In embodiments, the subject is an anti-PD-1 refractory subject or an anti-PD-1 resistant subject. In embodiments, the method of treating cancer is: (i) a method of increasing CD8-positive cells relative to the amount of regulatory T cells; (ii) a method of decreasing tumor volume; (iii) a method of enhancing anti-tumor immune memory; (iv) a method of treating a cancer tumor; or (v) two or more of the foregoing. In embodiments, the cancer is lung cancer, melanoma, breast cancer, colorectal cancer, bladder cancer, head and neck cancer, renal cell cancer, or prostate cancer. In embodiments, the subject is further administered a therapeutically effective amount of a chemotherapeutic agent, a therapeutically effective amount of an anti-CD73 antibody, or a combination thereof to treat the cancer.

The disclosure provides methods of treating cancer in a subject in need thereof by administering a therapeutically effective amount of an adenosine pathway inhibitor to the subject to treat the cancer; wherein the subject has: (i) an elevated level of adenosine A2A receptors when compared to a control; (ii) an elevated level of CD73 when compared to a control; and (iii) an elevated level of PD-L1 when compared to a control. In embodiments, the adenosine pathway inhibitor is an adenosine A2A receptor antagonist, an anti-CD73 compound, an anti-CD39 compound, or a combination of two or more thereof. In embodiments, the adenosine pathway inhibitor is an adenosine A2A receptor antagonist. In embodiments, the adenosine A2A receptor antagonist is a compound of Formula (I), Formula (II), Formula (III), Formula (IIIA), Formula (IIIB), or a pharmaceutically acceptable salt thereof. In embodiments, the subject has been previously treated with PD-1 pathway inhibitor therapy, such as a PD-1 inhibitor and/or a PD-L1 inhibitor. In embodiments, the subject is an anti-PD-1 refractory subject or an anti-PD-1 resistant subject. In embodiments, the method of treating cancer is: (i) a method of increasing CD8-positive cells relative to the amount of regulatory T cells; (ii) a method of decreasing tumor volume; (iii) a method of enhancing anti-tumor immune memory; (iv) a method of treating a cancer tumor; or (v) two or more of the foregoing. In embodiments, the cancer is lung cancer, melanoma, breast cancer, colorectal cancer, bladder cancer, head and neck cancer, renal cell cancer, or prostate cancer. In embodiments, the subject is further administered a therapeutically effective amount of a chemotherapeutic agent, a therapeutically effective amount of an anti-CD73 antibody, or a combination thereof to treat the cancer.

The disclosure provides methods of treating cancer in a subject in need thereof by administering a therapeutically effective amount of an adenosine pathway inhibitor to the subject to treat the cancer; wherein the subject has: (i) an elevated level of adenosine A2A receptors when compared to a control; and (ii) an elevated level of PD-L1 when compared to a control. In embodiments, the adenosine pathway inhibitor is an adenosine A2A receptor antagonist, an anti-CD73 compound, an anti-CD39 compound, or a combination of two or more thereof. In embodiments, the adenosine pathway inhibitor is an adenosine A2A receptor antagonist. In embodiments, the adenosine A2A receptor antagonist is a compound of Formula (I), Formula (II), Formula (III), Formula (IIIA), Formula (IIIB), or a pharmaceutically acceptable salt thereof. In embodiments, the subject has been previously treated with PD-1 pathway inhibitor therapy, such as a PD-1 inhibitor and/or a PD-L1 inhibitor. In embodiments, the subject is an anti-PD-1 refractory subject or an anti-PD-1 resistant subject. In embodiments, the method of treating cancer is: (i) a method of increasing CD8-positive cells relative to the amount of regulatory T cells; (ii) a method of decreasing tumor volume; (iii) a method of enhancing anti-tumor immune memory; (iv) a method of treating a cancer tumor; or (v) two or more of the foregoing. In embodiments, the cancer is lung cancer, melanoma, breast cancer, colorectal cancer, bladder cancer, head and neck cancer, renal cell cancer, or prostate cancer. In embodiments, the subject is further administered a therapeutically effective amount of a chemotherapeutic agent, a therapeutically effective amount of an anti-CD73 antibody, or a combination thereof to treat the cancer.

Provided herein are methods of treating cancer in a subject by: (i) measuring an adenosine A2A receptor level in a biological sample obtained from the subject, and (ii) administering a therapeutically effective amount of an adenosine pathway inhibitor to the subject to treat the cancer. In embodiments, the methods of treating cancer in a subject comprise: (i) measuring an adenosine A2A receptor level and a CD73 level in a biological sample obtained from the subject, and (ii) administering a therapeutically effective amount of an adenosine pathway inhibitor to the subject to treat the cancer. In embodiments, the methods of treating cancer in a subject comprise: (i) measuring an adenosine A2A receptor level, a CD73 level, and a PD-L1 level in a biological sample obtained from the subject, and (ii) administering a therapeutically effective amount of an adenosine pathway inhibitor to the subject to treat the cancer. In embodiments, the methods of treating cancer in a subject comprise: (i) measuring an adenosine A2A receptor level and a PD-L1 level in a biological sample obtained from the subject, and (ii) administering a therapeutically effective amount of an adenosine pathway inhibitor to the subject to treat the cancer. In embodiments, the biological sample is a tumor sample or a blood sample. In embodiments, the subject has been previously treated with PD-1 pathway inhibitor therapy, such as a PD-1 inhibitor and/or a PD-L1 inhibitor. In embodiments, the subject is an anti-PD-1 refractory subject or an anti-PD-1 resistant subject. In embodiments, the adenosine pathway inhibitor is an adenosine A2A receptor antagonist, an anti-CD73 compound, an anti-CD39 compound, or a combination of two or more thereof. In embodiments, the adenosine pathway inhibitor is an adenosine A2A receptor antagonist. In embodiments, the adenosine A2A receptor antagonist is a compound of Formula (I), Formula (II), Formula (III), Formula (IIIA), Formula (IIIB), or a pharmaceutically acceptable salt thereof. In embodiments, the method of treating cancer is: (i) a method of increasing CD8-positive cells relative to the amount of regulatory T cells; (ii) a method of decreasing tumor volume; (iii) a method of enhancing anti-tumor immune memory; (iv) a method of treating a cancer tumor; or (v) two or more of the foregoing. In embodiments, the cancer is lung cancer, melanoma, breast cancer, colorectal cancer, bladder cancer, head and neck cancer, renal cell cancer, or prostate cancer. In embodiments, the subject is further administered a therapeutically effective amount of a chemotherapeutic agent, a therapeutically effective amount of an anti-CD73 antibody, or a combination thereof to treat the cancer.

Provided here are methods to identify subjects who will be responsive to an adenosine pathway inhibitor, where the method comprises (i) obtaining a biological sample from the patient; and (ii) measuring an adenosine A2A receptor level in the biological sample; wherein if the adenosine A2A receptor level is elevated when compared to a control, the subject is identified as responsive to the adenosine pathway inhibitor. In embodiments, the methods to identify subjects who will be responsive to an adenosine pathway inhibitor comprise: (i) obtaining a biological sample from the patient; and (ii) measuring an adenosine A2A receptor level and a CD73 level in the biological sample; wherein if the adenosine A2A receptor level and the CD73 level are elevated when compared to a control, the subject is identified as responsive to the adenosine pathway inhibitor. In embodiments, the methods to identify subjects who will be responsive to an adenosine pathway inhibitor comprise: (i) obtaining a biological sample from the patient; and (ii) measuring an adenosine A2A receptor level, a CD73 level, and a PD-L1 level in the biological sample; wherein if the adenosine A2A receptor level, the CD73 level, and the PD-L1 level are elevated when compared to a control, the subject is identified as responsive to the adenosine pathway inhibitor. In embodiments, the methods to identify subjects who will be responsive to an adenosine pathway inhibitor comprise: (i) obtaining a biological sample from the patient; and (ii) measuring an adenosine A2A receptor level and a PD-L1 level in the biological sample; wherein if the adenosine A2A receptor level and the PD-L1 level are elevated when compared to a control, the subject is identified as responsive to the adenosine pathway inhibitor. In embodiments, the biological sample is a tumor sample or a blood sample. In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the adenosine pathway inhibitor is an adenosine A2A receptor antagonist, an anti-CD73 compound, an anti-CD39 compound, or a combination of two or more thereof. In embodiments, the adenosine pathway inhibitor is an adenosine A2A receptor antagonist. In embodiments, the adenosine A2A receptor antagonist is a compound of Formula (I), Formula (II), Formula (III), Formula (IIIA), Formula (IIIB), or a pharmaceutically acceptable salt thereof. In embodiments, the subject has cancer. In embodiments, the cancer is lung cancer, melanoma, breast cancer, colorectal cancer, bladder cancer, head and neck cancer, renal cell cancer, or prostate cancer.

Provided here are methods to select subjects for treatment with an adenosine pathway inhibitor, where the method comprises (i) obtaining a biological sample from the patient; and (ii) measuring an adenosine A2A receptor level in the biological sample; wherein if the adenosine A2A receptor level is elevated when compared to a control, the subject is selected for treatment with the adenosine pathway inhibitor. In embodiments, the method further comprises administering a therapeutically effective amount of the adenosine pathway inhibitor to treat cancer. In embodiments, the methods to select subjects for treatment with an adenosine pathway inhibitor comprise: (i) obtaining a biological sample from the patient; and (ii) measuring an adenosine A2A receptor level and a CD73 level in the biological sample; wherein if the adenosine A2A receptor level and the CD73 level are elevated when compared to a control, the subject is selected for treatment with the adenosine pathway inhibitor. In embodiments, the method further comprises administering a therapeutically effective amount of the adenosine pathway inhibitor to treat cancer. In embodiments, the methods to select subjects for treatment with an adenosine pathway inhibitor comprise: (i) obtaining a biological sample from the patient; and (ii) measuring an adenosine A2A receptor level, a CD73 level, and a PD-L1 level in the biological sample; wherein if the adenosine A2A receptor level, the CD73 level, and the PD-L1 level are elevated when compared to a control, the subject is selected for treatment with the adenosine pathway inhibitor. In embodiments, the method further comprises administering a therapeutically effective amount of the adenosine pathway inhibitor to treat cancer. In embodiments, the methods to select subjects for treatment with an adenosine pathway inhibitor comprise: (i) obtaining a biological sample from the patient; and (ii) measuring an adenosine A2A receptor level and a PD-L1 level in the biological sample; wherein if the adenosine A2A receptor level and the PD-L1 level are elevated when compared to a control, the subject is selected for treatment with the adenosine pathway inhibitor. In embodiments, the method further comprises administering a therapeutically effective amount of the adenosine pathway inhibitor to treat cancer. In embodiments, the biological sample is a tumor sample or a blood sample. In embodiments, the subject has been previously treated with PD-1 pathway inhibitor therapy, such as a PD-1 inhibitor and/or a PD-L1 inhibitor. In embodiments, the subject is an anti-PD-1 refractory subject or an anti-PD-1 resistant subject. In embodiments, the adenosine pathway inhibitor is an adenosine A2A receptor antagonist, an anti-CD73 compound, an anti-CD39 compound, or a combination of two or more thereof. In embodiments, the adenosine pathway inhibitor is an adenosine A2A receptor antagonist. In embodiments, the adenosine A2A receptor antagonist is a compound of Formula (I), Formula (II), Formula (III), Formula (IIIA), Formula (IIIB), or a pharmaceutically acceptable salt thereof. In embodiments, the subject has cancer. In embodiments, the cancer is lung cancer, melanoma, breast cancer, colorectal cancer, bladder cancer, head and neck cancer, renal cell cancer, or prostate cancer. In embodiments, the methods further comprise administering a therapeutically effective amount of a chemotherapeutic agent, a therapeutically effective amount of an anti-CD73 antibody, or a combination thereof to treat the cancer.

Provided herein are methods to determine whether a cancer patient expresses high adenosine A2A receptor levels by: (i) obtaining a biological sample from the patient; and (ii) measuring the adenosine A2A receptor levels in the biological sample. The method may further comprise administering a therapeutically effective amount of an adenosine pathway inhibitor. Provided herein are methods to determine whether a cancer patient expresses high adenosine A2A receptor levels and high CD73 levels by: (i) obtaining a biological sample from the patient; and (ii) measuring the adenosine A2A receptor levels and the CD73 levels in the biological sample. The method may further comprise administering a therapeutically effective amount of an adenosine pathway inhibitor. Provided herein are methods to determine whether a cancer patient expresses high adenosine A2A receptor levels, high CD73 levels, and high PD-L1 levels by: (i) obtaining a biological sample from the patient; and (ii) measuring the adenosine A2A receptor levels, the CD73 levels, and the PD-L1 levels in the biological sample. The method may further comprise administering a therapeutically effective amount of an adenosine pathway inhibitor. Provided herein are methods to determine whether a cancer patient expresses high adenosine A2A receptor levels and high PD-L1 levels by: (i) obtaining a biological sample from the patient; and (ii) measuring the adenosine A2A receptor levels and the PD-L1 levels in the biological sample. The method may further comprise administering a therapeutically effective amount of an adenosine pathway inhibitor. In embodiments, the biological sample is a tumor sample or a blood sample. In embodiments, the subject has been previously treated with PD-1 pathway inhibitor therapy, such as a PD-1 inhibitor and/or a PD-L1 inhibitor. In embodiments, the subject is an anti-PD-1 refractory subject or an anti-PD-1 resistant subject. In embodiments, the adenosine pathway inhibitor is an adenosine A2A receptor antagonist, an anti-CD73 compound, an anti-CD39 compound, or a combination of two or more thereof. In embodiments, the adenosine pathway inhibitor is an adenosine A2A receptor antagonist. In embodiments, the adenosine A2A receptor antagonist is a compound of Formula (I) or a pharmaceutically acceptable salt thereof. In embodiments, the adenosine A2A receptor antagonist is a compound of Formula (II) or a pharmaceutically Formula (I), Formula (II), Formula (III), Formula (IIIA), Formula (IIIB), or a pharmaceutically acceptable salt thereof. In embodiments, the subject has cancer. In embodiments, the cancer is lung cancer, melanoma, breast cancer, colorectal cancer, bladder cancer, head and neck cancer, renal cell cancer, or prostate cancer.

These and other embodiments of the disclosure are provided in more detail herein.

### BRIEF DESCRIPTION OF THE DRAWINGS

FIG. 1A-1C show the levels of adenosine A2A receptor expression (FIG. 1A), levels of CD73 expression (FIG. 1i), and levels of CD39 expression (FIG. 1C) in patients who were naïve to treatment with PD-1 pathway inhibitors, patients who were anti-PD-1 refractory, and patients who were anti-PD-1 resistant prior to treatment with the compound of Formula (III).

FIGS. 2A-2C show the levels of adenosine A2A receptor expression (FIG. 2A), levels of CD73 expression (FIG. 3B), and levels of CD39 expression (FIG. 2C) in patients who had renal cell cancer (RCC), non-small cell lung cancer (NSCLC), and other cancers (including bladder cancer, colorectal cancer, triple-negative breast cancer, melanoma, and prostate cancer) prior to treatment with the compound of Formula (III).

FIG. 3 is a graph showing tumor response to treatment with the compound of Formula (III) in patients expressing low levels of adenosine A2A receptors and patients expressing high levels of adenosine A2A receptors. RNA was extracted from pre-treatment tumor samples, and Nanostring was performed to arrive at the values described herein. The disease control rate was 0 of 11 patients who expressed low levels of adenosine A2A receptors, and was 9 of 26 patients who expressed high levels of adenosine A2A receptors. The objective response rate was 0 of II patients who expressed low levels of adenosine A2A receptors, and 1 of 26 patients who expressed high levels of adenosine A2A receptors. The graph shows the mean+/−standard deviation.

FIG. 4 is a graph showing tumor response to treatment with the compound of Formula (III) in patients expressing low levels of CD73 and patients expressing high levels of CD73. RNA was extracted from pre-treatment tumor samples, and Nanostring was performed to arrive at the values described herein. The disease control rate was 1 of 9 patients who expressed low levels of CD73, and was 8 of 28 patients who expressed high levels of CD73. The objective response rate was 0 of 9 patients who expressed low levels of CD73, and was 1 of 28 patients who expressed high levels of CD73. The graph shows the mean+/−standard deviation.

FIG. 5 is a graph showing tumor response to treatment with the compound of Formula (III) in patients expressing low levels of adenosine A2A receptors and/or CD73 and patients expressing high levels of both adenosine A2A receptors and CD73. RNA was extracted from pre-treatment tumor samples, and Nanostring was performed to arrive at the values described herein. The disease control rate was 8 of 21 patients who expressed low levels of adenosine A2A receptors and/or CD73, and was 1 of 16 patients who expressed high levels of both adenosine A2A receptors and CD73. The objective response rate was 1 of 21 patients who expressed low levels of adenosine A2A receptors and/or CD73, and was 0 of 16 patients who expressed high levels of both adenosine A2A receptors and CD73. The graph shows the mean+/−standard deviation.

FIG. 6 is a graph showing tumor response to treatment with the compound of Formula (III) in patients expressing low levels of PD-L1 mRNA and patients expressing high levels of PD-L1 mRNA. RNA was extracted from pre-treatment tumor samples, and Nanostring was performed to arrive at the values described herein. The disease control rate was 3 of 17 patients who expressed low levels of PD-L1 mRNA, and was 6 of 20 patients who expressed high levels of PD-L1 mRNA. The objective response rate was 0 of 17 patients who expressed low levels of PD-L1 mRNA, and was 1 of 20 patients who expressed high levels of PD-L1 mRNA. The graph shows the mean+/−standard deviation.

FIG. 7 is a graph showing tumor response to treatment with the compound of Formula (III) in patients having low levels of PD-L1 and patients having high levels of PD-L1. PD-L1 staining on immune cells was determined by immunohistochemistry using the SP142 antibody that detects PD-L1. The disease control rate was 7 of 24 patients who expressed low levels of PD-L1, and was 2 of 13 patients who expressed high levels of PD-L1. The objective response rate was 0 of 24 patients who expressed low levels of PD-L1, and was 1 of 13 patients who expressed high levels of PD-L1. The graph shows the mean+/−standard deviation.

FIGS. 8A-8H show log₂-fold changes in immunohistochemistry and gene expression (i.e., mRNA) by Nanostring on on-treatment biopsies compared to pre-treatment biopsies and identifies pharmacodynamics changes that are observed in CD73-high tumors treated with a compound of Formula (III). FIG. 8A compares CD8 infiltration, measured by IHC, in patients expressing low levels and high levels of CD73 (where the p=0.005) after treatment with the compound of Formula (III). FIG. 8B compares PD-L1 inflammation infiltration, measured by IHC, in patients expressing low levels and high levels of CD73 (p=0.15) after treatment with the compound of Formula (III). FIG. 8C compares CXCL9 (chemokine C-X-C motif ligand 9) gene expression in patients expressing low levels and high levels of CD73 (p=0.02) after treatment with the compound of Formula (III). FIG. 8D compares CXCL10 (chemokine C-X-C motif ligand 10) gene expression in patients expressing low levels and high levels of CD73 (p=0.07) after treatment with the compound of Formula (III). FIG. 8E compares GZMA (granzyme A) gene expression in patients expressing low levels and high levels of CD73 (p=0.07) after treatment with the compound of Formula (III). FIG. 8F compares GZMB (granzyme B) gene expression in patients expressing low levels and high levels of CD73 (p=0.04) after treatment with the compound of Formula (III). FIG. 8G compares IDO1 (indoleamine 2,3-dioxygenase 1) gene expression in patients expressing low levels and high levels of CD73 (p=0.01) after treatment with the compound of Formula (III). FIG. 8H compares LAG3 (lymphocyte activation gene 3) gene expression in patients expressing low levels and high levels of CD73 (p=0.16) after treatment with the compound of Formula (III).

### DETAILED DESCRIPTION

### Definitions

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

“Disease control rate” refers to the patients that had a reduction in tumor size or volume; no change in tumor size or volume; and a confirmed tumor growth of <20% in the sum of the longest dimensions of their assessed target tumor lesions.

“Objective response rate” refers to the patients that had greater than 30% reduction in the sum of the longest dimensions of their assessed target tumor lesions.

“Partial response” refers to at least a 30% reduction in tumor size or volume (e.g., in the sum of the longest dimensions of their assessed target tumor lesions).

The terms “adenosine A2A receptor” or “A2A receptor” or “A2A adenosine receptor” include any of the recombinant or naturally-occurring forms of the adenosine A2A receptor (ADORA2A) or variants or homologs thereof that maintain ADORA2A protein activity (e.g. within at least 50%, 80%, 90%, 95%, 96%, 97%, 98%, 99% or 100% activity compared to ADORA2A). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ADORA2A polypeptide. In embodiments, ADORA2A is the protein as identified by the NCBI sequence reference GI:5921992, homolog or functional fragment thereof.

An “adenosine A2A receptor antagonist” or “A2A receptor antagonist” refer to a substance capable of detectably lowering expression or activity level of an adenosine A2A receptor compared to a control. The inhibited expression or activity of the A2A receptor can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. An “antagonist” is a compound or small molecule that inhibits an A2A receptor e.g., by binding, partially or totally blocking stimulation, decrease, prevent, or delay activation, or inactivate, desensitize, or down-regulate signal transduction, gene expression or enzymatic activity necessary for A2A activity. In embodiments, the A2A receptor antagonist is a compound or a small molecule. In embodiments, the A2A receptor antagonist is an antibody. In embodiments, the adenosine pathway inhibitor is a compound of Formula (I), Formula (II), Formula (III), Formula (IIIA), Formula (IIIB), or a pharmaceutically acceptable salt of any of the foregoing.

A “compound of Formula (I)” is an adenosine A2A receptor antagonist and refers to a compound having the following structure:

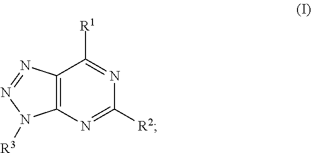

wherein the substituents R¹, R², and R³ are as defined herein.

A “compound of Formula (II)” is an adenosine A2A receptor antagonist and refers to a compound having the following structure:

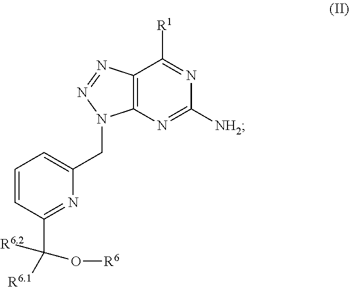

wherein the substituents R, R⁶, R^{6.1}, and R^{6.2} are as defined herein.

A “compound of Formula (III),” also known as CPI-444, is an adenosine A2A receptor antagonist and refers to a compound having the following structure:

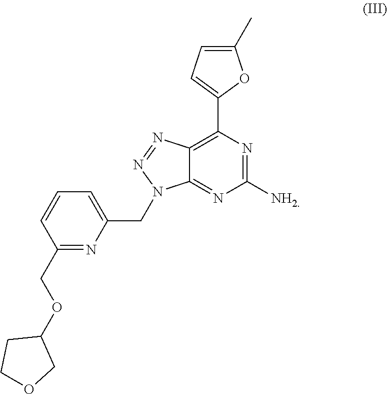 [SMILES: Cc1ccc(-c2nc(N)nc3c2nnn3Cc2cccc(COC3CCOC3)n2)o1]

In embodiments, the compound of Formula (III) is a compound of Formula (IIIA). In embodiments, the compound of Formula (III) is a compound of Formula (III). In embodiments, the compound of Formula (III) is a mixture of the compounds of Formula (IIIA) and (IIIB).

A “compound of Formula (IIIA)” refers to a compound having the following structure:

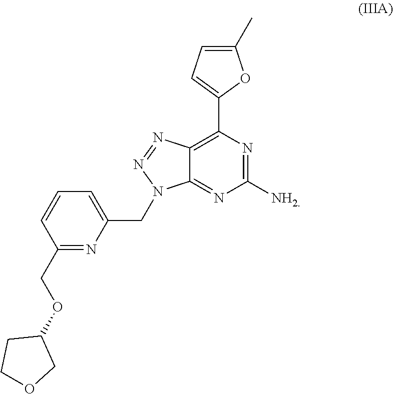

A “compound of Formula (IIIB)” refers to a compound having the following structure:

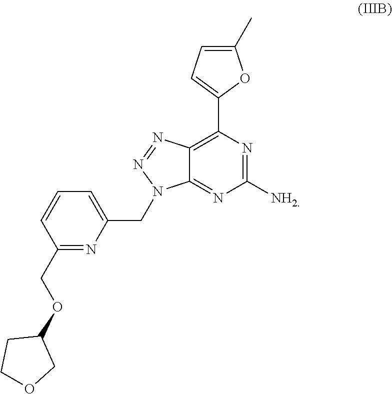

“Adenosine A2A receptor levels” as referred to herein is the level of adenosine A2A receptors expressed by a tumor. The levels can be measured by genes, mRNA, or proteins in a biological sample.

“An elevated level of adenosine A2A receptors” as referred to herein is an elevated level of adenosine A2A receptor genes expressed by a tumor in a subject when compared to a control. Adenosne A2A receptor levels can be measured from biological samples, such as a tumor sample (e.g., resected, biopsy) or a blood sample (e.g., peripheral blood), obtained from a subject. A tumor can be a primary tumor or a metastasic tumor. A tumor as provided herein is a cellular mass including cancer cells and non-cancer cells. The non-cancer cells forming part of a tumor may be stromal cells, and immune cells (e.g., T cells, dendritic cells, B cells, macrophages). Thus, the elevated level of adenosine may be expressed by a non-cancer cell (e.g., a stromal cell) or a cancer cell (e.g., a malignant T cell). The term is further defined herein.

“Adenosine pathway inhibitor” as provided herein refers to a substance capable of detectably lowering expression of or activity level of the adenosine signaling pathway compared to a control. The inhibited expression or activity of the adenosine signaling pathway can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. An “inhibitor” is a compound or small molecule that inhibits the adenosine signaling pathway e.g., by binding, partially or totally blocking stimulation of the adenosine signaling pathway, decrease, prevent, or delay activation of the adenosine signaling pathway, or inactivate, desensitize, or down-regulate signal transduction, gene expression or enzymatic activity of the adenosine signaling pathway. In embodiments, the adenosine pathway inhibitor inhibits adenosine activity or expression. In embodiments, the adenosine pathway inhibitor is a compound or a small molecule. In embodiments, the adenosine pathway inhibitor is an antibody. In embodiments, the adenosine pathway inhibitor is an anti-CD73 compound. In embodiments, the adenosine pathway inhibitor is an anti-CD39 compound. In embodiments, the adenosine pathway inhibitor comprises an anti-CD73 compound and an anti-CD39 compound. In embodiments, the adenosine pathway inhibitor comprises an adenosine A2A receptor antagonist and an anti-CD73 compound. In embodiments, the adenosine pathway inhibitor comprises an adenosine A2A receptor antagonist and an anti-CD39 compound. In embodiments, the adenosine pathway inhibitor comprises an adenosine A2A receptor antagonist, an anti-CD73 compound, and an anti-CD39 compound. In embodiments, the adenosine pathway inhibitor is a adenosine receptor antagonist. In embodiments, the adenosine pathway inhibitor is a adenosine A2A receptor antagonist. In embodiments, the adenosine pathway inhibitor is a compound of Formula (I), Formula (II), Formula (III), Formula (IIIA), Formula (IIIB), or a pharmaceutically acceptable salt of any of the foregoing.

“Subject responsive to an adenosine pathway inhibitor” refers to a subject that responds to treatment when administered an adenosine pathway inhibitor. “Responsive” and “responds” indicate that: (i) the subject has an increase over baseline of one or more of CD8+ cell infiltration, T cell activation, interferon-gamma pathway gene expression, and T cell clone expansion; (ii) a cancerous tumor does not grow in size or volume over time; (iii) a cancerous tumor decreases in size or volume over time; (iv) a cancerous tumor does not metastasize; or (v) a combination of two or more of the foregoing. In embodiments, a subject responsive to an adenosine pathway inhibitor has an increase over baseline of one or more of CD8+ cell infiltration, T cell activation, interferon-gamma pathway gene expression, T cell clone expansion, or a combination thereof, where such increase is at least 1.5-fold, or at least 2-fold, or at least 2.5-fold over baseline or a control. In embodiments, a subject responsive to an adenosine pathway inhibitor shows a decrease (i.e., reduction) in tumor size or volume after treatment compared to baseline or a control. In embodiments, a subject responsive to an adenosine pathway inhibitor shows at least a 5% reduction in tumor size or volume after treatment compared to baseline or a control. In embodiments, a subject responsive to an adenosine pathway inhibitor shows at least a 10% reduction in tumor size or volume after treatment compared to baseline or a control. In embodiments, a subject responsive to an adenosine pathway inhibitor shows at least a 20% reduction in tumor size or volume after treatment compared to baseline or a control. In embodiments, a subject responsive to an adenosine pathway inhibitor shows at least a 25% reduction in tumor size or volume after treatment compared to baseline or a control. In embodiments, a subject responsive to an adenosine pathway inhibitor shows at least a 30% reduction in tumor size or volume after treatment compared to baseline or a control. In embodiments, a subject responsive to an adenosine pathway inhibitor shows at least a 35% reduction in tumor size or volume after treatment compared to baseline or a control. In embodiments, a subject responsive to an adenosine pathway inhibitor shows at least a 40% reduction in tumor size or volume after treatment compared to baseline or a control. In embodiments, a subject responsive to an adenosine pathway inhibitor shows at least a 45% reduction in tumor size or volume after treatment compared to baseline or a control. In embodiments, a subject responsive to an adenosine pathway inhibitor shows at least a 50% reduction in tumor size or volume after treatment compared to baseline or a control. In embodiments, a subject responsive to an adenosine pathway inhibitor shows at least a 55% reduction in tumor size or volume after treatment compared to baseline or a control. In embodiments, a subject responsive to an adenosine pathway inhibitor shows at least a 60% reduction in tumor size or volume after treatment compared to baseline or a control.

A “CD73 protein” or “CD73 antigen” as referred to herein includes any of the recombinant or naturally-occurring forms of the Cluster of Differentiation 73 (CD73) also known as 5′-nucleotidase (5′-NT) or ecto-5′-nucleotidase or variants or homologs thereof that maintain CD73 nucleotidase activity (e.g. within at least 50%, 80%, 90%, 95%, 96%, 97%, 98%, 99% or 100% activity compared to CD73). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD73 protein. In embodiments, the CD73 protein is substantially identical to the protein identified by the UniProt reference number 21589 or a variant or homolog having substantial identity thereto. In embodiments, the CD73 protein is substantially identical to the protein identified by the UniProt reference number Q61503 or a variant or homolog having substantial identity thereto.

“CD73 levels” as referred to herein is the level of CD73 expressed by a tumor.

“An elevated level of CD73” as referred to herein is an elevated level of CD73 expressed (e.g., mRNA, proteins) by a tumor in a subject when compared to a control. CD73 levels can be measured from biological samples, such as a tumor sample (e.g., resected, biopsy) or a blood sample (e.g., peripheral blood), obtained from a subject. A tumor can be a primary tumor or a metastasis. A tumor as provided herein is a cellular mass including cancer cells and non-cancer cells. The non-cancer cells forming part of a tumor may be stromal cells, and immune cells (e.g., T cells, dendritic cells, B cells, macrophages). Thus, the elevated level of CD73 may be expressed by a non-cancer cell (e.g., a stromal cell) or a cancer cell (e.g., a malignant T cell). The term is further defined herein.

An “anti-CD73 compound” refers to any compound (e.g., small molecule, peptide, protein, antibody) capable of binding to CD73 or otherwise inhibiting the ability of CD73 to perform normal functions in the adenosine pathway. In embodiments, the anti-CD73 compound is an anti-CD73 antibody. Exemplary anti-CD73 antibodies include 1E9 antibodies, IgG1 antibodies, humanized 1E9 antibodies, humanized IgG1 antibodies, and the like.

An “anti-CD39 compound” refers to any compound (e.g., small molecule, peptide, protein, antibody) capable of binding to CD39 or otherwise inhibiting the ability of CD39 to perform normal functions in the adenosine pathway. In embodiments, the anti-CD39 compound is an anti-CD39 antibody.

A “PD-1 protein” or “PD-1” as referred to herein includes any of the recombinant or naturally-occurring forms of the programmed cell death protein 1 (PD-1) also known as cluster of differentiation 279 (CD 279) or variants or homologs thereof that maintain PD-1 protein activity (e.g. within at least 50%, 80%, 90%, 95%, 96%, 97%, 98%, 99% or 100% activity compared to PD-1 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-1 protein. In embodiments, the PD-1 protein is substantially identical to the protein identified by the UniProt reference number Q15116 or a variant or homolog having substantial identity thereto. In embodiments, the PD-1 protein is substantially identical to the protein identified by the UniProt reference number Q02242 or a variant or homolog having substantial identity thereto.

A “PD-L1 protein” or “PD-L” as referred to herein includes any of the recombinant or naturally-occurring forms of the programmed death-ligand 1 (PD-L1) also known as cluster of differentiation 274 (CD 274) or variants or homologs thereof that maintain PD-L1 protein activity (e.g. within at least 50%, 80%, 90%, 95%, 96%, 97%, 98%, 99% or 100% activity compared to PD-L1 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-L1 protein. In embodiments, the PD-L1 protein is substantially identical to the protein identified by the UniProt reference number Q9NZQ7 or a variant or homolog having substantial identity thereto. In embodiments, the PD-L1 protein is substantially identical to the protein identified by the UniProt reference number Q9EP73 or a variant or homolog having substantial identity thereto.

“PD-1 pathway inhibitor” as provided herein refers to a substance capable of detectably lowering expression of or activity level of the PD-1 signaling pathway compared to a control. The inhibited expression or activity of the PD-1 signaling pathway can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. An “inhibitor” is a compound or small molecule that inhibits the PD-1 signaling pathway e.g., by binding, partially or totally blocking stimulation of the PD-1 pathway, decrease, prevent, or delay activation of the PD-1 pathway, or inactivate, desensitize, or down-regulate signal transduction, gene expression or enzymatic activity of the PD-1 pathway. In embodiments, the PD-1 pathway inhibitor inhibits PD-1 activity or expression. In embodiments, the PD-1 pathway inhibitor is a compound or a small molecule. In embodiments, the PD-1 pathway inhibitor is an antibody.

In embodiments, the PD-1 pathway inhibitor is a programmed death-ligand 1 (PD-L1) inhibitor or a PD-1 inhibitor. A PD-L1 inhibitor as provided herein is a substance that, at least in part, partially or totally blocks stimulation, decreases, prevents, or delays activation, or inactivates, desensitizes, or down-regulates signal transduction of PD-1. A PD-1 inhibitor as provided herein is a substance that, at least in part, partially or totally blocks stimulation, decreases, prevents, or delays activation, or inactivates, desensitizes, or down-regulates signal transduction of PD-1.

The term “inhibitor,” “inhibition,” “inhibit,” “inhibiting” and the like in reference to a protein-inhibitor (e.g., an adenosine pathway inhibitor, a PD-1 pathway inhibitor) interaction means negatively affecting (e.g., decreasing) the activity or function of the protein (e.g., decreasing the activity of an A2A receptor, CD73, a PD-1 protein or PD-L1 protein) relative to the activity or function of the protein in the absence of the inhibitor (e.g., an adenosine pathway inhibitor, a PD-1 pathway inhibitor). In some embodiments, inhibition refers to reduction of a disease or symptoms of disease (e.g., cancer). Thus, inhibition includes, at least in part, partially or totally blocking stimulation, decreasing, preventing, or delaying activation, or inactivating, desensitizing, or down-regulating signal transduction or enzymatic activity or the amount of a protein (e.g., an A2A receptor, CD73, a PD-1 protein, PD-L1 protein). Similarly an “inhibitor” is a compound or protein that inhibits an A2A receptor or a PD-1 protein or PD-L1 protein, e.g., by binding, partially or totally blocking, decreasing, preventing, delaying, inactivating, desensitizing, or down-regulating activity (e.g., an A2A receptor activity, CD73, a PD-1 protein activity, a PD-L1 protein activity).

“Previously treated with PD-1 pathway inhibitor therapy” refers to a subject that had been treated with PD-1 pathway inhibitor therapy in the past or treated with PD-1 pathway inhibitor therapy prior to treatment with an adenosine pathway inhibitor. In embodiments, the subject previously treated with PD-1 pathway inhibitor therapy was previously treated with a PD-1 inhibitor. In embodiments, the subject previously treated with PD-1 pathway inhibitor therapy was previously treated with a PD-L1 inhibitor. In embodiments, the subject previously treated with PD-1 pathway inhibitor therapy was previously treated with a PD-1 inhibitor and a PD-L1 inhibitor. In embodiments, the subject previously treated with PD-1 pathway inhibitor therapy is an “anti-PD-1 refractory subject” or a “refractory subject;” wherein the PD-1 pathway inhibitor therapy was a PD-1 inhibitor, a PD-L1 inhibitor, or a combination thereof. In embodiments, the subject previously treated with PD-1 pathway inhibitor therapy is an “anti-PD-1 resistant subject” or a “resistant subject;” wherein the PD-1 pathway inhibitor therapy was a PD-1 inhibitor, a PD-L1 inhibitor, or a combination thereof. In embodiments, the subject was responsive to prior PD-1 pathway inhibitor therapy, wherein the PD-1 pathway inhibitor was a PD-1 inhibitor, a PD-L1 inhibitor, or a combination thereof.

“Anti-PD-1 refractory subject” or “refractory subject” or “O-refractory subject” refer to cancer patients who are unresponsive to PD-1 pathway inhibitor therapy, such as treatment with PD-1 inhibitors and/or PD-L1 inhibitors. Generally, a refractory subject has been treated with a PD-1 pathway inhibitor (e.g., PD-1 inhibitor, PD-L1 inhibitor) for one month, two months, or three months, and was unresponsive to treatment with the PD-1 pathway inhibitor. In aspects, an anti-PD-1 refractory subject is an anti-PD-L1 refractory subject. Where the cancer patient is unresponsive to PD-1 pathway inhibitor therapy the patient shows less than 20% reduction in tumor size or volume after administration of PD-1 pathway inhibitor relative to a control. In embodiments, an anti-PD-1 refractory subject shows less than 10% reduction in tumor size or volume after administration of PD-1 pathway inhibitor relative to a control. In embodiments, an anti-PD-1 refractory subject shows less than 5% reduction in tumor size or volume after administration of PD-1 pathway inhibitor relative to a control. In embodiments, an anti-PD-1 refractory subject shows less than 1% reduction in tumor size or volume after administration of PD-1 pathway inhibitor relative to a control. In embodiments, an anti-PD-1 refractory subject shows less than 0.5% reduction in tumor size or volume after administration of PD-1 pathway inhibitor relative to a control. In embodiments, an anti-PD-1 refractory subject shows less than 0.1% reduction in tumor size or volume after administration of PD-1 pathway inhibitor relative to a control. In embodiments, an anti-PD-1 refractory subject shows no reduction in tumor size or volume after administration of PD-1 pathway inhibitor relative to a control. In embodiments, an anti-PD-1 refractory subject shows an increase in tumor size or volume after administration of PD-1 pathway inhibitor relative to a control.

“Anti-PD-1 resistant subject” or “resistant subject” or “IO-resistant subject” refer to cancer patients who are initially responsive to PD-1 pathway inhibitor therapy, but then became resistant to PD-1 pathway inhibitor therapy (e.g., PD-1 inhibitor, PD-L1 inhibitor). “Resistant subjects” have been treated with PD-1 pathway inhibitor therapy for more than three months. The “anti-PD-1 resistant subject” initially showed some benefits from the PD-1 pathway inhibitor therapy, where the benefits could have been: (i) an increase over baseline of one or more of CD8+ cell infiltration, T cell activation, interferon-gamma pathway gene expression, and T cell clone expansion; (ii) a cancerous tumor that did not grow in size or volume; (iii) a cancerous tumor that decreased in size or volume; (iv) a cancerous tumor that did not metastasize; or (v) a combination of two or more of the foregoing. After initially showing a benefit to treatment, the “resistant subject” then became unresponsive to the PD-1 pathway inhibitor therapy, such as treatment with PD-1 inhibitors and/or PD-L1 inhibitors. In aspects, an anti-PD-1 resistant subject is an anti-PD-L1 resistant subject. Where the cancer patient is unresponsive to PD-1 pathway inhibitor therapy, the patient shows less than 20% reduction in tumor size or volume after administration of PD-1 pathway inhibitor relative to a control. Thus, in embodiments, an anti-PD-1 resistant subject shows less than 20% reduction in tumor size or volume after administration of PD-1 pathway inhibitor relative to a control. In embodiments, an anti-PD-1 resistant subject shows less than 10% reduction in tumor size or volume after administration of PD-1 pathway inhibitor relative to a control. In embodiments, an anti-PD-1 resistant subject shows less than 5% reduction in tumor size or volume after administration of PD-1 pathway inhibitor relative to a control. In embodiments, an anti-PD-1 resistant subject shows less than 1% reduction in tumor size or volume after administration of PD-1 pathway inhibitor relative to a control. In embodiments, an anti-PD-1 resistant subject shows less than 0.5% reduction in tumor size or volume after administration of PD-1 pathway inhibitor relative to a control. In embodiments, an anti-PD-1 resistant subject shows less than 0.1% reduction in tumor size or volume after administration of PD-1 pathway inhibitor relative to a control. In embodiments, an anti-PD-1 resistant subject shows no reduction in tumor size or volume after administration of PD-1 pathway inhibitor relative to a control. In embodiments, an anti-PD-1 resistant subject shows an increase in tumor size or volume after administration of PD-1 pathway inhibitor relative to a control.

“Subject naïve to PD-1 pathway inhibitor therapy” or “IO-naïve” refer to a subject that had not previously been treated with PD-1 pathway inhibitor therapy, such as PD-1 inhibitors or PD-L1 inhibitors.

“Subject responsive to prior PD-1 pathway inhibitor therapy” refers to a subject that had been treated with PD-1 pathway inhibitor therapy prior to the initiation of treatment with the adenosine pathway inhibitor, wherein the subject had been responsive to treatment with the PD-1 pathway inhibitor therapy. “Responsive” and “responds” indicate that: (i) the cancerous tumor had not grown in size or volume over time; (iii) the cancerous tumor had decreased in size or volume over time; (iv) the cancerous tumor had not metastasize; or (v) a combination of two or more of the foregoing. In embodiments, a “subject responsive to prior PD-1 pathway inhibitor therapy” had shown a decrease (i.e., reduction) in tumor size or volume during/after treatment compared to baseline or a control. In embodiments, a subject responsive to prior PD-1 pathway inhibitor therapy had shown at least a 20% reduction or at least a 25% reduction in tumor size or volume during/after treatment compared to baseline or a control. In embodiments, a subject responsive to prior PD-1 pathway inhibitor therapy had shown at least a 30% reduction or at least a 35% reduction in tumor size or volume during/after treatment compared to baseline or a control. In embodiments, a subject responsive to prior PD-1 pathway inhibitor therapy had shown at least a 40% reduction or at least a 45% reduction in tumor size or volume during/after treatment compared to baseline or a control. In embodiments, a subject responsive to prior PD-1 pathway inhibitor had shown at least a 50% reduction or at least a 60% reduction in tumor size or volume during/after treatment compared to baseline or a control.

“Biological sample” refers to any biological sample taken from a subject. Biological samples include blood, plasma, serum, tumors, tissue, cells, and the like. In embodiments, the biological sample is a blood sample. In embodiments, the biological sample is a peripheral blood sample. In embodiments, the biological sample is a tumor sample. In embodiments, the biological sample is a primary tumor sample. In embodiments, the biological sample is a metastatic tumor sample. In embodiments, the biological sample is a resected tumor sample. In embodiments, the biological sample is a tumor biopsy sample. In embodiments, the biological sample is a resected tumor sample from a primary tumor. In embodiments, the biological sample is a resected tumor sample from a metastisic tumor. In embodiments, the biological sample is a tumor biopsy sample from a primary tumor. In embodiments, the biological sample is a tumor biopsy sample from a metastisic tumor. Biological samples can be taken from a subject by methods known in the art, and can be analyzed by methods known in the art.

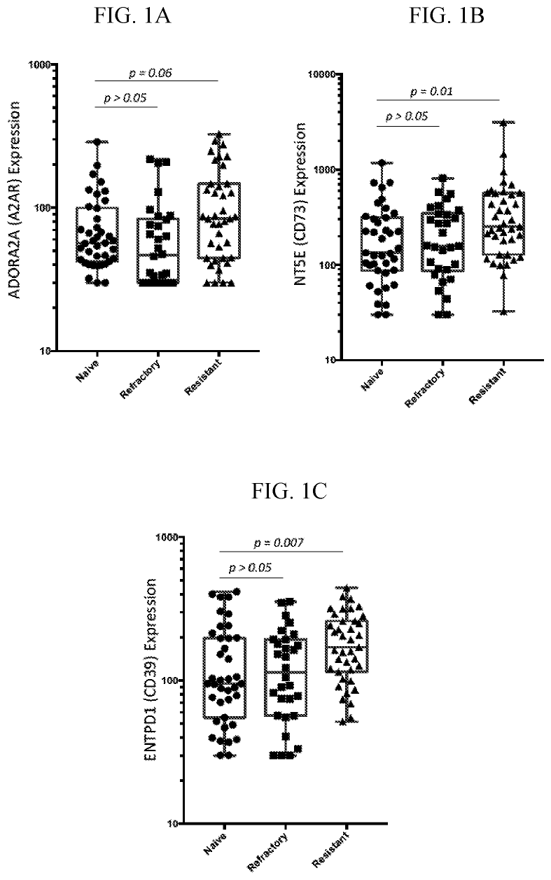

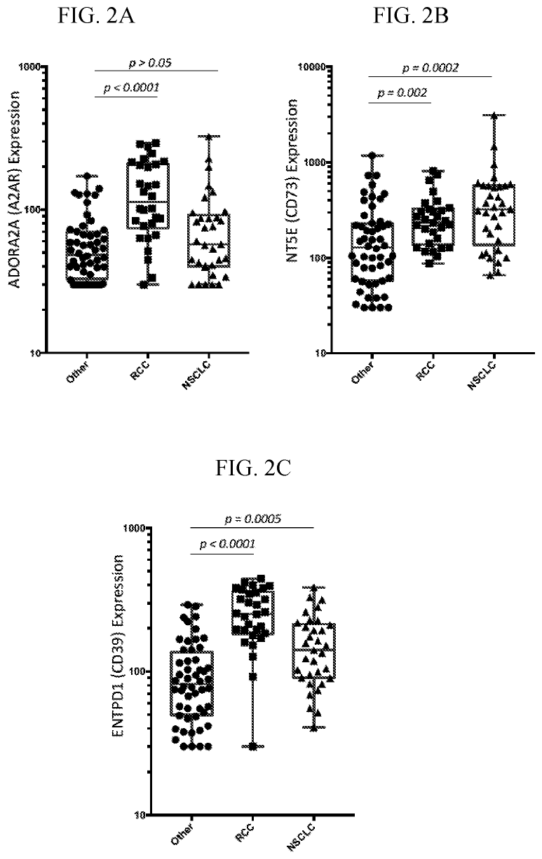

A “control” sample or value refers to a sample that serves as a reference, usually a known reference, for comparison to a test sample. For example, a test sample can be taken from a patient suspected of having a given disease (cancer) and compared to samples from a known cancer patient, or a known normal (non-disease) individual. A control can also represent an average value gathered from a population of similar individuals, e.g., cancer patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters. In embodiments, a control is a negative control. In embodiments, such as some embodiments relating to detecting the level of expression or infiltration, a control comprises the average amount of expression (e.g., protein or mRNA) of infiltration (e.g., number or percentage of cells in a population of cells) in a population of subjects (e.g., with cancer) or in a healthy or general population. In embodiments, the control comprises an average amount (e.g. percentage or number of infiltrating cells or amount of expression) in a population in which the number of subjects (n) is 5 or more, 6 or more, 7 or more, 8 or more, 9 or more, 10 or more, 25 of more, 50 or more, 100 or more, 1000 or more, 5000 or more, or 10000 or more. In embodiments, the control is a standard control. In embodiments, the control is a population of cancer subjects who are anti-PD-1 resistant or anti-PD-1 refractory. In embodiments, the control is a tumor sample from a population of cancer subjects who are anti-PD-1 resistant or anti-PD-1 refractory. In embodiments, the control is the results shown in FIGS. 1A-1C. In embodiments, the control is the results shown in FIGS. 2A-2C. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

A “CD8+T lymphocyte” or “CD8 T cell” or “CD8-positive T cell” and the like as referred to herein is a lymphocyte that expresses the CD8 glycoprotein on its surface. Examples of CD8 T cells include cytotoxic T cells and natural killer cells. In one embodiment, a CD8 T cell is a cytotoxic T cell. In embodiments, a CD8 T cell is a suppressor T cell. CD8 comprises an alpha-chain and a beta-chain. The term “CD8a” as provided herein refers to the alpha-chain of CD8, and includes homologues and isoforms thereof. Non-limiting amino acid sequences for CD8a include NCBI Accession Nos. AAH25715.1, NP_001759.3, and NP_741969.1, which are all incorporated herein by reference. Non-limiting nucleotide sequences for CD8a include NCBI Accession Nos. NR_027353.1, NM_001768.6, NM_171827.3, and NM_001145873.1, which are all incorporated herein by reference. In embodiments, a CD8a protein is a protein having amino acids in the sequence of one of the NCBI Accession numbers for CD8a disclosed herein, or an isoform or homologue thereof. In embodiments, a CD8a protein includes any protein having amino acids in the sequence of any one of one of the NCBI Accession numbers for CD8a disclosed herein, or an isoform or homologue thereof.

A “memory T cell” is a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen. In embodiments, the memory T cell is a CD45RA-negative CD4 T cell. In embodiments, the memory T cell is a CD45RA-negative CD8 T cell.

The term “CD45RA” as provided herein refers to the CD45 Receptor antigen also known as Protein tyrosine phosphatase, receptor type, C (PTPRC). Non-limiting amino acid sequences for CD45RA include GENBANK® Accession Nos. NP_002829.3, NP_563578.2, NP_563578.2, and NP_002829.3, which are all incorporated herein by reference. CD45RA is expressed on naïve T cells, as well as on CD8- and CD4-expressing effector cells. After antigen interaction, T cells gain expression of CD45RO and lose expression of CD45RA. Thus, either CD45RA or CD45RO is used to generally differentiate the naïve from memory T cell populations. Thus, a “CD45RA-negative CD8 T cell” as provided herein is a CD8 T cell which lacks expression of detectable amounts of CD45RA. In embodiments, the CD45RA-negative CD8 T cell is a memory T cell. A “CD45RA-negative CD4 T cell” as provided herein is a CD4 T cell which lacks expression of detectable amounts of CD45RA. In embodiments, the CD45RA-negative CD4 T cell is a memory T cell. In embodiments, the CD45RA-negative CD8 T cell is a memory T cell.

A “regulatory T cell” or “suppressor T cell” is a lymphocyte which modulates the immune system, maintains tolerance to self-antigens, and prevents autoimmune disease. Regulatory T cells express the CD4, FOXP3, and CD25 and are thought to be derived from the same lineage as naïve CD4 cells.

The term “anti-tumor immune memory” as provided herein refers to the ability of the immune system of a subject to recognize (memorize) previously encountered tumor antigen. Once the tumor antigen has been recognized, the immune system reproduces (e.g., through T cell activation and proliferation) and can mount a faster and stronger immune response than the first time it responded to the same tumor antigen.

The term “global immune activation” as provided herein refers to the activation of immune cells of the adaptive immune system in a subject. Examples of immune cells activated during global immune activation are without limitation, antigen presenting cells (macrophages, dendritic cells), B cells and T cells. The activation may occur through recognition of a previously encountered antigen (tumor antigen) or it may occur through encounter of a novel (not previously encountered) antigen (tumor antigen).

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety. The terms “peptidyl” and “peptidyl moiety” means a monovalent peptide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M). (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

As used herein “treating a cancer tumor” means preventing an increase in size or volume of the cancer tumor. In embodiments, the cancer tumor is a solid tumor. In embodiments, treating a cancer tumor includes decreasing the size of volume of a cancer tumor. In embodiments, treating a cancer tumor includes eliminating the cancer tumor altogether. In embodiments, a cancer tumor is eliminated when it is not detectable by an imaging test such as magnetic resonance imaging (MRI), a positron emission tomography (PET) scan, X-ray computed tomography (CT), ultrasound, or single-photon emission computed tomography (SPECT). In embodiments, treating a cancer tumor further comprises reducing or preventing metastasis of the cancer tumor.

The terms “disease” or “condition” refer to a state of being or health status of a patient or subject capable of being treated with a compound, pharmaceutical composition, or method provided herein. In embodiments, the disease is cancer, such as lung cancer (e.g., non-small cell lung cancer), melanoma (e.g., malignant melanoma), renal cell cancer, breast cancer (e.g., triple negative breast cancer), colorectal cancer (e.g., microsatellite instable colorectal cancer), bladder cancer, prostate cancer (e.g., metastatic castration resistant prostrate cancer, castration resistant prostrate cancer), or a head and neck cancer.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer (e.g., squamous cell carcinoma of the head and neck), kidney cancer (e.g., renal cell carcinoma), myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiform, glioma, melanoma, prostate cancer, castration-resistant prostate cancer, metastatic castration resistant prostate cancer, breast cancer, triple negative breast cancer, glioblastoma, ovarian cancer, lung cancer, squamous cell carcinoma (e.g., head, neck, or esophagus), colorectal cancer (e.g., microsatellite instable colorectal cancer), leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiform, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

As used herein, the terms “metastasis,” “metastatic,” “metastatic tumor,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, the anti-cancer agent is not a PD-1 pathway inhibitor, i.e., the anti-cancer agent is not a PD-1 inhibitor or a PD-L1 inhibitor. In embodiments, an anti-cancer agent is a chemotherapeutic. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds or platinum containing agents (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (GLEEVEC™), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. paclitaxel), Taxotere™, compounds comprising the taxane skeleton, Erbulozole (i.e. R-55104), Dolastatin 10 (i.e. DLS-10 and NSC-376128), mivobulin isethionate (i.e. as CI-980), vincristine, NSC-639829, discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), Vincristine sulfate, Cryptophycin 52 (i.e. LY-355703), Vitilevuamide, Tubulysin A, Canadensol, Centaureidin (i.e. NSC-106969), Oncocidin A1 (i.e. BTO-956 and DIME), Fijianolide B, Laulimalide, Narcosine (also known as NSC-5366), Nascapine, Hemiasterlin, Vanadocene acetylacetonate, Monsatrol, lnanocine (i.e. NSC-698666), Eleutherobins (such as Desmethyleleutherobin, Desaetyleleutherobin, lsoeleutherobin A, and Z-Eleutherobin), Caribaeoside, Caribaeolin, Halichondrin B, Diazonamide A, Taccalonolide A, Diozostatin, (−)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In ⁹⁰Y or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (IRESSA™), erlotinib (TARCEVA™), cetuximab (ERBUTUX™) lapatinib (TYKERB™), panitumumab (VECTIBIX™), vandetanib (CAPRELSA™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, hormonal therapies, or the like.

A “patient” or “subject” includes both humans and other animals, particularly mammals. Thus, the methods are applicable to both human therapy and veterinary applications. In embodiments, the patient is a mammal. In embodiments, the patient is a companion animal, such as a dog or a cat. In embodiments, the patient is human.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O—is equivalent to —OCH₂—.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the disclosure. “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched non-cyclic chain, or combinations thereof, including at least one carbon atom and at least one heteroatom (e.g. O, N, P, Si, and S), and wherein the nitrogen and sulfur atoms may optionally be oxidized, and the nitrogen heteroatom may optionally be quaternized. The heteroatom(s) (e.g. O, N, P, S, and Si) may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, 1H-1,2,4-triazolyl-5(4H)-one, 4H-1,2,4-triazolyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. A heterocycloalkyl moiety may include one ring heteroatom (e.g., O, N, S, Si, or P). A heterocycloalkyl moiety may include two optionally different ring heteroatoms (e.g., O, N, S, Si, or P). A heterocycloalkyl moiety may include three optionally different ring heteroatoms (e.g., O, N, S, Si, or P). A heterocycloalkyl moiety may include four optionally different ring heteroatoms (e.g., O, N, S, Si, or P). A heterocycloalkyl moiety may include five optionally different ring heteroatoms (e.g., O, N, S, Si, or P). A heterocycloalkyl moiety may include up to 8 optionally different ring heteroatoms (e.g., O, N, S, Si, or P).

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “alkylsulfonyl,” as used herein, means a moiety having the formula —S(O)₂R′—, where R′ is a substituted or unsubstituted alkyl group as defined above. R′ may have a specified number of carbons (e.g., “C₁-C₄ alkylsulfonyl”).

Each of the above terms (e.g., “alkyl”, “heteroalkyl”, “cycloalkyl”, “heterocycloalkyl”, “aryl”, and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″ —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R′R″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ-X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties: (A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and (B) alkyl, heteroalkyl, cycloalkyl, heterocycloalkyl, aryl, heteroaryl, substituted with at least one substituent selected from: (i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, heteroaryl, substituted with at least one substituent selected from: (a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₅ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

The term “pharmaceutically acceptable salts” is meant to include salts of the active compounds that are prepared with relatively nontoxic acids or bases, depending on the particular substituents found on the compounds described herein. When compounds of the disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the disclosure contain both basic and acidic functionalities that allow the compounds to be converted into either base or acid addition salts. Other pharmaceutically acceptable carriers known to those of skill in the art are suitable for the disclosure. Salts tend to be more soluble in aqueous or other protonic solvents than are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the disclosure may exist as salts, such as with pharmaceutically acceptable acids. The disclosure includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Provided herein are agents (e.g. compounds, drugs, therapeutic agents) that may be in a prodrug form. Prodrugs of the compounds described herein are those compounds that readily undergo chemical changes under select physiological conditions to provide the final agents (e.g. compounds, drugs, therapeutic agents). Additionally, prodrugs can be converted to agents (e.g. compounds, drugs, therapeutic agents) by chemical or biochemical methods in an ex vivo environment. Prodrugs described herein include compounds that readily undergo chemical changes under select physiological conditions to provide agents (e.g. compounds, drugs, therapeutic agents) to a biological system (e.g. in a subject).

Certain compounds of the disclosure can exist in unsolvated forms as well as solvated forms, including hydrated forms. In general, the solvated forms are equivalent to unsolvated forms and are encompassed within the scope of the disclosure. Certain compounds of the disclosure may exist in multiple crystalline or amorphous forms. In general, all physical forms are equivalent for the uses contemplated by the disclosure and are intended to be within the scope of the disclosure.

As used herein, the term “salt” refers to acid or base salts of the compounds used in the methods of the disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the disclosure. The compounds of the disclosure do not include those which are known in art to be too unstable to synthesize and/or isolate. The disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another. It will be apparent to one skilled in the art that certain compounds may exist in tautomeric forms, and all such tautomeric forms are within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the disclosure, whether radioactive or not, are encompassed within the scope of the disclosure.

The symbol “ [symbol] ” denotes the point of attachment of a chemical moiety to the remainder of a molecule or chemical formula.

In embodiments, a compound as described herein may include multiple instances of R² and/or other variables. In such embodiments, each variable may optional be different and be appropriately labeled to distinguish each group for greater clarity. For example, where each R² is different, they may be referred to, for example, as R^{2.1}, R^{2.2}, R^{2.3}, and/or R^{2.4} respectively, wherein the definition of R² is assumed by R^{2.1}, R^{2.2}, R^{2.3}, and/or R^{2.4}. The variables used within a definition of R² and/or other variables that appear at multiple instances and are different may similarly be appropriately labeled to distinguish each group for greater clarity. In some embodiments, the compound is a compound described herein (e.g., in an aspect, embodiment, example, claim, table, scheme, drawing, or figure).

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. For example, where a moiety herein is R¹²-substituted or unsubstituted alkyl, a plurality of R¹² substituents may be attached to the alkyl moiety wherein each R¹² substituent is optionally different. Where an R-substituted moiety is substituted with a plurality R substituents, each of the R-substituents may be differentiated herein using a prime symbol (′) such as R′, R″, etc. For example, where a moiety is R¹²-substituted or unsubstituted alkyl, and the moiety is substituted with a plurality of R¹² substituents, the plurality of R¹² substituents may be differentiated as R^{12′}, R¹²″, R¹²′″, etc. In embodiments, the plurality of R substituents is 3. In embodiments, the plurality of R substituents is 2.

In embodiments, a compound as described herein may include multiple instances of R¹, R², R³, R⁴, R⁵, R⁶, R⁷, R⁹, R¹⁰, R¹¹, R¹², R¹³, R¹⁴ and/or other variables. In such embodiments, each variable may optional be different and be appropriately labeled to distinguish each group for greater clarity. For example, where each R¹, R², R³, R⁴, R⁵, R, R⁷, R⁹, R¹⁰, R, R¹², R¹³, and/or R¹⁴, is different, they may be referred to, for example, as R¹, R^{1.2}, R^{1.3}, R^{1.4}, R², R^{2.2}, R^{2.3}, R^{2.4}, R^{3.1}, R^{3.2}, R^{3.3}, R^{3.4}, R^{4.1}, R^{4.2}, R^{4.3}, R^{4.4}, R^{5.1}, R^{5.2}, R^{5.3}, R^{5.4}, R^{6.1}, R^{6.2}, R^{6.3}, R^{6.4}, R^{7.1}, R^{7.2}, R^{7.3}, R^{7.4}, R^{9.1}, R^{9.2}, R^{9.3}, R^{9.4}, R^{10.1}, R^{10.2}, R^{10.3}, R^{10.4}, R^{11.1}, R^{11.2}, R^{11.3}, R^{11.4}, R^{12.1}, R^{12.2}, R^{12.3}, R^{12.4}, R^{13.1}, R^{13.2}, R^{13.3}, R^{13.4}, R^{14.1}, R^{14.2}, R^{14.3}, and/or R^{14.4}, respectively, wherein the definition of R¹ is assumed by R^{1.1}, R^{1.2}, R^{1.3}, and/or R^{1.4}, the definition of R² is assumed by R^{2.1}, R^{2.2}, R^{2.3}, and/or R^{2.4}, the definition of R³ is assumed by R^{3.1}, R^{3.2}, R^{3.3}, and/or R^{3.4}, the definition of R⁴ is assumed by R⁴, R⁴², R^{4.3}, and/or R⁴⁴, the definition of R⁵ is assumed by R^{5.1}, R^{5.2}, R^{5.3}, and/or R^{5.4}, the definition of R⁶ is assumed by R^{6.1}, R^{6.2}, R^{6.3}, and/or R^{6.4}, the definition of R⁷ is assumed by R^{7.1}, R^{7.2}, R^{7.3}, and/or R^{7.4}, the definition of R⁹ is assumed by R^{9.1}, R^{9.2}, R^{9.3}, and/or R^{9.4}, the definition of R¹⁰ is assumed by R^{10.1}, R^{10.2}, R^{10.3}, and/or R^{10.4}, the definition of R¹¹ is assumed by R^{11.1}, R^{11.2}, R^{11.3}, and/or R^{11.4}, the definition of R¹² is assumed by R^{12.1}, R^{12.2}, R^{12.3}, and/or R^{12.4}, the definition of R¹³ is assumed by R^{13.1}, R^{13.2}, R^{13.3}, and/or R^{13.4}, the definition of R¹⁴ is assumed by R^{14.1}, R^{14.2}, R^{14.3}, and/or R^{14.4}. The variables used within a definition of R¹, R², R³, R⁴, R⁵, R⁶, R⁷, R⁹, R¹⁰, R¹¹, R¹², R¹³ and/or R¹⁴, and/or other variables that appear at multiple instances and are different may similarly be appropriately labeled to distinguish each group for greater clarity.

Descriptions of compounds of the disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

In embodiments, the adenosine pathway inhibitor is a purine receptor antagonist. In embodiments, the adenosine pathway inhibitor is an adenosine A2A receptor antagonist. In embodiments, the adenosine pathway inhibitor is a thienopyrimidine compound. In embodiments, the adenosine pathway inhibitor is any one of the compounds disclosed in U.S. Pat. Nos. 9,120,807, 8,450,328 and 8,354,415, which are incorporated by reference herein in their entirety.

In embodiments, the A2A receptor antagonist is a compound of Formula (I) or a pharmaceutically acceptable salt thereof:

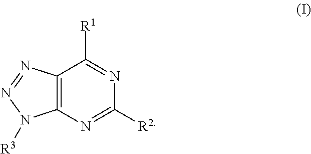

In formula (I), R¹ is independently hydrogen, halogen, —CX^{a}₃, —CN, —SO₂Cl, —SOₙ₁R⁹, —SOᵥ₁NR⁹R¹⁰, —NHNH₂, —ONR⁹R¹⁰, —NHC═(O)NHNH₂, —NHC═(O)NR⁹R¹⁰, —N(O)ₘ₁, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is independently hydrogen, halogen, —CX^{b}₃, —CN, —SO₂Cl, —SOₙ₂R¹¹, —SOᵥ₂NR¹¹R¹², —NHNH₂, —ONR¹¹R¹², —NHC═(O)NHNH₂, —NHC═(O)NR¹¹R¹², —N(O)ₘ₂, —NR¹¹R¹², —NH—O—R¹¹, —C(O)R¹¹, —C(O)—OR¹¹, —C(O)NR¹¹R¹², —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is independently hydrogen, halogen, —CX^{c}₃, —CN, —SO₂Cl, —SOₙ₃R¹³, —SOᵥ₃NR¹³R¹⁴, —NHNH₂, —ONR¹³R¹⁴, —NHC═(O)NHNH₂, —NHC═(O)NR¹³R¹⁴, —N(O)ₘ₃, —NR¹³R¹⁴, —NH—O—R¹³, —C(O)R¹³, —C(O)—OR¹³, —C(O)NR¹³R¹⁴, —OR¹³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁹, R¹⁰, R¹¹, R¹², R¹³ and R¹⁴ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁹, R¹⁰, R¹¹, R¹², R¹³ and R¹⁴ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X^{a}, X^{b} and X^{c} are independently —F, —Cl, —Br, or —I.

The symbols n₁, n₂ and n₃ are independently an integer from 0 to 4. In embodiments, n₁ is 0. In embodiments, n₁ is 1. In embodiments, n₁ is 3. In embodiments, n₁ is 4. In embodiments, n₂ is 0. In embodiments, n₂ is 1. In embodiments, n₂ is 3. In embodiments, n₂ is 4. In embodiments, n₃ is 0. In embodiments, n₃ is 1. In embodiments, n₃ is 3. In embodiments, n₃ is 4.

The symbols m₁, m₂ and m₃ are independently an integer from 1 to 2. In embodiments, m₁ is 0. In embodiments, m₁ is 1. In embodiments, m₁ is 2. In embodiments, m₂ is 0. In embodiments, m₂ is 1. In embodiments, m₂ is 2. In embodiments, m₃ is 0. In embodiments, m₃ is 1. In embodiments, m₂ is 2.

The symbols v₁, v₂ and v₃ are independently an integer from 1 to 2. In embodiments, v₁ is 0. In embodiments, v₁ is 1. In embodiments, v₁ is 2. In embodiments, v₂ is 0. In embodiments, v₂ is 1. In embodiments, v₂ is 2. In embodiments, v₃ is 0. In embodiments, v₃ is 1. In embodiments, v₃ is 2.

In embodiments, R¹ is independently hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1A}-substituted or unsubstituted alkyl, R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl. R¹ may be R^{1A}-substituted or unsubstituted (e.g., C₁-C₂₀ or C₁-C₆) alkyl, R^{1A}-substituted or unsubstituted (e.g., 2 to 20 membered or 2 to 6 membered) heteroalkyl, R^{1A}-substituted or unsubstituted (e.g., C₃-C₈ or C₅-C₇) cycloalkyl, R^{1A}-substituted or unsubstituted (e.g., 3 to 8 membered or 3 to 6 membered) heterocycloalkyl, R^{1A}-substituted or unsubstituted (e.g., C₅-C₁₀ or C₅-C₆) aryl, or R^{1A}-substituted or unsubstituted (e.g., 5 to 10 membered or 5 to 6 membered) heteroaryl.

In embodiments, R^{1A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl, or R^{1B}-substituted or unsubstituted heteroaryl. R^{1A} may be R^{1B}-substituted or unsubstituted (e.g., C₁-C₂₀ or C₁-C₆) alkyl, R^{1B}-substituted or unsubstituted (e.g., 2 to 20 membered or 2 to 6 membered) heteroalkyl, R^{1B}-substituted or unsubstituted (e.g., C₃-C₈ or C₅-C₇) cycloalkyl, R^{1B}-substituted or unsubstituted (e.g., 3 to 8 membered or 3 to 6 membered) heterocycloalkyl, R^{1B}-substituted or unsubstituted (e.g., C₅-C₁₀ or C₅-C₆) aryl, or R^{1B}-substituted or unsubstituted (e.g., 5 to 10 membered or 5 to 6 membered) heteroaryl.

In embodiments, R^{1B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl, or R^{1C}-substituted or unsubstituted heteroaryl. R^{1B} may be R^{1C}-substituted or unsubstituted (e.g., C₁-C₂₀ or C₁-C₆) alkyl, R^{1C}-substituted or unsubstituted (e.g., 2 to 20 membered or 2 to 6 membered) heteroalkyl, R^{1C}-substituted or unsubstituted (e.g., C₃-C₈ or C₅-C₇) cycloalkyl, R^{1C}-substituted or unsubstituted (e.g., 3 to 8 membered or 3 to 6 membered) heterocycloalkyl, R^{1C}-substituted or unsubstituted (e.g., C₅-C₁₀ or C₅-C₆) aryl, or R^{1C}-substituted or unsubstituted (e.g., 5 to 10 membered or 5 to 6 membered) heteroaryl.

R^{1C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. R^{1C} may be independently unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

In embodiments, R¹ is independently R^{1A}-substituted or unsubstituted alkyl, R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl. In embodiments, R¹ is R^{1A}-substituted or unsubstituted (e.g., 5 to 10 membered or 5 to 6 membered) heteroaryl. In embodiments, R¹ is unsubstituted 5 to 6 membered heteroaryl. In embodiments, R¹ is R^{1A}-substituted 5 to 6 membered heteroaryl. In embodiments, R¹ is unsubstituted 5 membered heteroaryl. In embodiments, R¹ is R^{1A}-substituted 5 membered heteroaryl. In embodiments, R¹ is R^{1A}-substituted furanyl.

In embodiments, R^{1A} is R^{1B}-substituted or unsubstituted (e.g., C₁-C₂₀ or C₁-C₆) alkyl. In embodiments, R^{1A} is R^{1B}-substituted C₁-C₆ alkyl. In embodiments, R^{1A} is unsubstituted C₁-C₆ alkyl. In embodiments, R^{1A} is R^{1B}-substituted C₁-C₄ alkyl. In embodiments, R^{1A} is unsubstituted C₁-C₄ alkyl. In embodiments, R^{1A} is R^{1B}-substituted C₁-C₃ alkyl. In embodiments, R^{1A} is unsubstituted C₁-C₃ alkyl. In embodiments, R^{1A} is methyl.

In embodiments, R² is independently hydrogen, halogen, —CX^{b}₃, —CN, —SO₂Cl, —SOₙ₂R¹¹, —SOᵥ₂NR¹¹R¹², —NHNH₂, —ONR¹¹R¹², —NHC═(O)NHNH₂, —NHC═(O)NR¹¹R¹², —N(O)ₘ₂, —NR¹¹R¹², —NH—O—R¹¹, —C(O)R¹¹, —C(O)—OR¹¹, —C(O)NR¹¹R¹², or —OR¹¹. In embodiments of the methods provided herein, R² is independently hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In embodiments, R² is —NR¹¹R¹². In embodiments, R¹¹ and R¹² are independently hydrogen or substituted or unsubstituted (e.g., C₁-C₂₀ or C₁-C₆) alkyl. In embodiments, R¹¹ and R¹² are independently substituted or unsubstituted C₁-C₆ alkyl. In embodiments, R¹¹ and R¹² are independently substituted or unsubstituted C₁-C₄ alkyl. In embodiments, R¹¹ and R¹² are independently substituted or unsubstituted C₁-C₃ alkyl. In embodiments, R¹¹ and R¹² are independently unsubstituted C₁-C₆ alkyl. In embodiments, R¹¹ and R¹² are independently substituted or unsubstituted C₁-C₄ alkyl. In embodiments, R¹¹ and R¹² are independently unsubstituted C₁-C₃ alkyl. In embodiments, R¹¹ and R¹² are independently hydrogen.

In embodiments, R³ is independently hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁴-substituted or unsubstituted alkyl, R⁴-substituted or unsubstituted heteroalkyl, R⁴-substituted or unsubstituted cycloalkyl, R⁴-substituted or unsubstituted heterocycloalkyl, R⁴-substituted or unsubstituted aryl, or R⁴-substituted or unsubstituted heteroaryl. R³ may be R⁴-substituted or unsubstituted (e.g., C₁-C₂₀ or C₁-C₆) alkyl, R⁴-substituted or unsubstituted (e.g., 2 to 20 membered or 2 to 6 membered) heteroalkyl, R⁴-substituted or unsubstituted (e.g., C₃-C₈ or C₅-C₇) cycloalkyl, R⁴-substituted or unsubstituted (e.g., 3 to 8 membered or 3 to 6 membered) heterocycloalkyl, R⁴-substituted or unsubstituted (e.g., C₅-C₁₀ or C₅-C₆) aryl, or R⁴-substituted or unsubstituted (e.g., 5 to 10 membered or 5 to 6 membered) heteroaryl.

R⁴ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁵-substituted or unsubstituted alkyl, R⁵-substituted or unsubstituted heteroalkyl, R⁵-substituted or unsubstituted cycloalkyl, R⁵-substituted or unsubstituted heterocycloalkyl, R⁵-substituted or unsubstituted aryl, or R⁵-substituted or unsubstituted heteroaryl. R⁴ may be R⁵-substituted or unsubstituted (e.g., C₁-C₂₀ or C₁-C₆) alkyl, R⁵-substituted or unsubstituted (e.g., 2 to 20 membered or 2 to 6 membered) heteroalkyl, R⁵-substituted or unsubstituted (e.g., C₃-C₈ or C₅-C₇) cycloalkyl, R⁵-substituted or unsubstituted (e.g., 3 to 8 membered or 3 to 6 membered) heterocycloalkyl, R⁵-substituted or unsubstituted (e.g., C₅-C₁₀ or C₅-C₆) aryl, or R⁵-substituted or unsubstituted (e.g., 5 to 10 membered or 5 to 6 membered) heteroaryl.

R⁵ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁶-substituted or unsubstituted alkyl, R⁶-substituted or unsubstituted heteroalkyl, R⁶-substituted or unsubstituted cycloalkyl, R⁶-substituted or unsubstituted heterocycloalkyl, R⁶-substituted or unsubstituted aryl, or R⁶-substituted or unsubstituted heteroaryl. R⁵ may be R⁶-substituted or unsubstituted (e.g., C₁-C₂₀ or C₁-C₆) alkyl, R⁶-substituted or unsubstituted (e.g., 2 to 20 membered or 2 to 6 membered) heteroalkyl, R⁶-substituted or unsubstituted (e.g., C₃-C₈ or C₅-C₇) cycloalkyl, R⁶-substituted or unsubstituted (e.g., 3 to 8 membered or 3 to 6 membered) heterocycloalkyl, R⁶-substituted or unsubstituted (e.g., C₅-C₁₀ or C₅-C₆) aryl, or R⁶-substituted or unsubstituted (e.g., 5 to 10 membered or 5 to 6 membered) heteroaryl.

R⁶ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁷-substituted or unsubstituted alkyl, R⁷-substituted or unsubstituted heteroalkyl, R⁷-substituted or unsubstituted cycloalkyl, R⁷-substituted or unsubstituted heterocycloalkyl, R⁷-substituted or unsubstituted aryl, or R⁷-substituted or unsubstituted heteroaryl. R⁶ may be R⁷-substituted or unsubstituted (e.g., C₁-C₂₀ or C₁-C₆) alkyl, R⁷-substituted or unsubstituted (e.g., 2 to 20 membered or 2 to 6 membered) heteroalkyl, R⁷-substituted or unsubstituted (e.g., C₃-C₈ or C₅-C₇) cycloalkyl, R⁷-substituted or unsubstituted (e.g., 3 to 8 membered or 3 to 6 membered) heterocycloalkyl, R⁷-substituted or unsubstituted (e.g., C₅-C₁₀ or C₅-C₆) aryl, or R⁷-substituted or unsubstituted (e.g., 5 to 10 membered or 5 to 6 membered) heteroaryl.

In embodiments, R³ is independently hydrogen, halogen, R⁴-substituted or unsubstituted alkyl, R⁴-substituted or unsubstituted heteroalkyl, R⁴-substituted or unsubstituted cycloalkyl, R⁴-substituted or unsubstituted heterocycloalkyl, R⁴-substituted or unsubstituted aryl, or R⁴-substituted or unsubstituted heteroaryl. In embodiments, R³ is independently R⁴-substituted or unsubstituted (e.g., C₁-C₂₀ or C₁-C₆) alkyl. In embodiments, R³ is independently R⁴-substituted or unsubstituted C₁-C₆ alkyl. In embodiments, R³ is independently R⁴-substituted or unsubstituted C₁-C₅ alkyl. In embodiments, R³ is independently R⁴-substituted or unsubstituted C₁-C₄ alkyl. In embodiments, R³ is independently R⁴-substituted or unsubstituted C₁-C₃ alkyl. In embodiments, R³ is independently unsubstituted C₁-C₆ alkyl. In embodiments, R³ is independently unsubstituted C₁-C₅ alkyl. In embodiments, R³ is independently R⁴-unsubstituted C₁-C₄ alkyl. In embodiments, R³ is independently unsubstituted C₁-C₃ alkyl. In embodiments, R³ is independently R⁴-substituted C₁-C₆ alkyl. In embodiments, R³ is independently R⁴-substituted C₁-C₅ alkyl. In embodiments, R³ is independently R⁴-substituted C₁-C₄ alkyl. In embodiments, R³ is independently R⁴-substituted C₁-C₃ alkyl. In embodiments, R³ is R⁴-substituted C₁ alkyl.

In embodiments, R⁴ is R⁵-substituted or unsubstituted (e.g., C₁-C₂₀ or C₁-C₆) alkyl, R⁵-substituted or unsubstituted (e.g., 2 to 20 membered or 2 to 6 membered) heteroalkyl, R⁵-substituted or unsubstituted (e.g., C₃-C₈ or C₅-C₇) cycloalkyl, R⁵-substituted or unsubstituted (e.g., 3 to 8 membered or 3 to 6 membered) heterocycloalkyl, R⁵-substituted or unsubstituted (e.g., C₅-C₁₀ or C₅-C₆) aryl, or R⁵-substituted or unsubstituted (e.g., 5 to 10 membered or 5 to 6 membered) heteroaryl. In embodiments, R⁴ is R⁵-substituted or unsubstituted 5 to 6 membered heteroaryl. In embodiments, R⁴ is R⁵-substituted or unsubstituted 6 membered heteroaryl. In embodiments, R⁴ is unsubstituted 6 membered heteroaryl. In embodiments, R⁴ is R⁵-substituted 6 membered heteroaryl. In embodiments, R⁴ is R⁵-substituted pyridinyl.

In embodiments, R⁵ is R⁶-substituted or unsubstituted (e.g., C₁-C₂₀ or C₁-C₆) alkyl, R⁶-substituted or unsubstituted (e.g., 2 to 20 membered or 2 to 6 membered) heteroalkyl, R⁶-substituted or unsubstituted (e.g., C₃-C₈ or C₅-C₇) cycloalkyl, R⁶-substituted or unsubstituted (e.g., 3 to 8 membered or 3 to 6 membered) heterocycloalkyl, R⁶-substituted or unsubstituted (e.g., C₅-C₁₀ or C₅-C₆) aryl, or R⁶-substituted or unsubstituted (e.g., 5 to 10 membered or 5 to 6 membered) heteroaryl. In embodiments, R⁵ is R⁶-substituted or unsubstituted 2 to 6 membered heteroalkyl. In embodiments, R⁵ is R⁶-substituted or unsubstituted 2 to 5 membered heteroalkyl. In embodiments, R⁵ is R⁶-substituted or unsubstituted 2 to 4 membered heteroalkyl. n embodiments, R⁵ is R⁶-substituted or unsubstituted 2 to 3 membered heteroalkyl. In embodiments, R⁵ is R⁶-substituted or unsubstituted 2 membered heteroalkyl. In embodiments, R⁵ is unsubstituted 2 to 6 membered heteroalkyl. In embodiments, R⁵ is unsubstituted 2 to 5 membered heteroalkyl. In embodiments, R⁵ is unsubstituted 2 to 4 membered heteroalkyl. In embodiments, R⁵ unsubstituted 2 to 3 membered heteroalkyl. In embodiments, R⁵ is unsubstituted 2 membered heteroalkyl. In embodiments, R⁵ is R⁶-substituted 2 to 6 membered heteroalkyl. In embodiments, R⁵ is R⁶-substituted 2 to 5 membered heteroalkyl. In embodiments, R⁵ is R⁶-substituted 2 to 4 membered heteroalkyl. In embodiments, R⁵ is R-substituted 2 to 3 membered heteroalkyl. In embodiments, R⁵ is R⁶-substituted 2 membered heteroalkyl.

In embodiments, R⁶ is R⁷-substituted or unsubstituted (e.g., C₁-C₂₀ or C₁-C₆) alkyl, R⁷-substituted or unsubstituted (e.g., 2 to 20 membered or 2 to 6 membered) heteroalkyl, R⁷-substituted or unsubstituted (e.g., C₃-C₅ or C₅-C₇) cycloalkyl, R⁷-substituted or unsubstituted (e.g., 3 to 8 membered or 3 to 6 membered) heterocycloalkyl, R⁷-substituted or unsubstituted (e.g., C₅-C₁₀ or C₅-C₆) aryl, or R⁷-substituted or unsubstituted (e.g., 5 to 10 membered or 5 to 6 membered) heteroaryl. In embodiments, R⁶ is R⁷-substituted or unsubstituted 3 to 6 membered heterocycloalkyl. In embodiments, R⁶ is R⁷-substituted or unsubstituted 5 membered heterocycloalkyl. In embodiments, R⁶ is R⁷-substituted 5 membered heterocycloalkyl. In embodiments, R⁶ is unsubstituted 5 membered heterocycloalkyl. In embodiments, R⁶ is unsubstituted tetrahydrofuranyl.

In embodiments of the methods provided herein, R⁹, R¹⁰, R¹¹, R¹², R¹³ and R¹⁴ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

In embodiments, R¹ is R^{1A}-substituted furanyl. In one further embodiment, R^{1A} is methyl. In another further embodiment, R² is —NR¹¹R¹². In another further embodiment, R¹¹ and R¹² are independently hydrogen. In yet another further embodiment, R³ is R⁴-substituted C₁ alkyl. In another further embodiment, R⁴ is R⁵-substituted pyridinyl. In yet another further embodiment, R⁵ is R⁶-substituted 2 membered heteroalkyl. In another further embodiments, R⁶ is unsubstituted tetrahydrofuranyl.

In embodiments, the A2A receptor antagonist is a compound of Formula (II) or a pharmaceutically acceptable salt thereof:

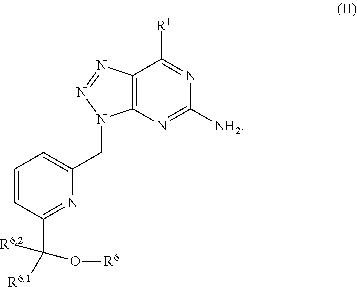

In formula (II), R⁶, R^{6.1} and R^{6.2} are independently hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁶, R^{6.1} and R^{6.2} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{6.1} and R^{6.2} are hydrogen and R6 is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{6.1} and R^{6.2} are hydrogen and R6 is substituted or unsubstituted heterocycloalkyl. In embodiments, R^{6.1} and R^{6.2} are hydrogen and R⁶ is unsubstituted heterocycloalkyl. In embodiments, R¹ is substituted (e.g. with an unsubstituted C₁-C₅ alkyl) or unsubstituted heteroaryl. In embodiments, R¹ is substituted (e.g. with an unsubstituted C₁-C₅ alkyl) or unsubstituted furanyl. In embodiments, R¹ is methyl-substituted furanyl.

In formula (II), R¹ and R⁶ are as described above (e.g., R⁶ may be R⁷-substituted or unsubstituted 3 to 6 membered heterocycloalkyl and R¹ may be R^{1A}-substituted 5 to 6 membered heteroaryl). Thus, in embodiments, R⁶ is unsubstituted tetrahydrofuranyl and R¹ is R^{1A}-substituted furanyl.

In formula (II), R^{6.1} may be independently hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{7.1}-substituted or unsubstituted alkyl, R^{7.1}-substituted or unsubstituted heteroalkyl, R^{7.1}-substituted or unsubstituted cycloalkyl, R^{7.1}-substituted or unsubstituted heterocycloalkyl, R^{7.1}-substituted or unsubstituted aryl, or R^{7.1}-substituted or unsubstituted heteroaryl. R^{6.1} may be R^{7.1}-substituted or unsubstituted (e.g., C₁-C₂₀ or C₁-C₆) alkyl, R^{7.1}-substituted or unsubstituted (e.g., 2 to 20 membered or 2 to 6 membered) heteroalkyl, R⁷¹-substituted or unsubstituted (e.g., C₃-C₈ or C₅-C₇) cycloalkyl, R^{7.1}-substituted or unsubstituted (e.g., 3 to 8 membered or 3 to 6 membered) heterocycloalkyl, R^{7.1}-substituted or unsubstituted (e.g., C₅-C₁₀ or C₅-C₆) aryl, or R^{7.1}-substituted or unsubstituted (e.g., 5 to 10 membered or 5 to 6 membered) heteroaryl. In embodiments, R^{6.1} is R^{7.1}-substituted or unsubstituted C₁-C₆ alkyl. In embodiments, R^{6.1} is R^{7.1}-substituted or unsubstituted C₁-C₅ alkyl. In embodiments, R^{6.1} is R^{7.1}-substituted or unsubstituted C₁-C₄ alkyl. In embodiments, R^{6.1} is R^{7.1}-substituted or unsubstituted C₁-C₃ alkyl. In embodiments, R^{6.1} is R^{7.1}-substituted C₁-C₆ alkyl. In embodiments, R^{6.1} is R^{7.1}-substituted C₁-C₅ alkyl. In embodiments, R^{6.1} is R^{7.1}-substituted C₁-C₄ alkyl. In embodiments, R^{6.1} is R⁷¹-substituted C₁-C₃ alkyl. In embodiments, R^{6.1} is unsubstituted C₁-C₆ alkyl. In embodiments, R^{6.1} is unsubstituted C₁-C₅ alkyl. In embodiments, R^{6.1} is unsubstituted C₁-C₄ alkyl. In embodiments, R^{6.1} is unsubstituted C₁-C₃ alkyl. In embodiments, R^{6.1} is unsubstituted methyl.

R^{6.2} is independently hydrogen, halogen, ═O, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{7.2}-substituted or unsubstituted alkyl, R^{7.2}-substituted or unsubstituted heteroalkyl, R^{7.2}-substituted or unsubstituted cycloalkyl, R^{7.2}-substituted or unsubstituted heterocycloalkyl, R^{7.2}-substituted or unsubstituted aryl, or R^{7.2}-substituted or unsubstituted heteroaryl. R^{6.2} may be R^{7.2}-substituted or unsubstituted (e.g., C₁-C₂₀ or C₁-C₆) alkyl, R^{7.2}-substituted or unsubstituted (e.g., 2 to 20 membered or 2 to 6 membered) heteroalkyl, R^{7.2}-substituted or unsubstituted (e.g., C₃-C₈ or C₅-C₇) cycloalkyl, R^{7.2}-substituted or unsubstituted (e.g., 3 to 8 membered or 3 to 6 membered) heterocycloalkyl, R^{7.2}-substituted or unsubstituted (e.g., C₅-C₁₀ or C₅-C₆) aryl, or R^{7.2}-substituted or unsubstituted (e.g., 5 to 10 membered or 5 to 6 membered) heteroaryl. In embodiments, R^{6.2} is R^{7.2}-substituted or unsubstituted C₁-C₆ alkyl. In embodiments, R^{6.2} is R^{7.2}-substituted or unsubstituted C₁-C₅ alkyl. In embodiments, R^{6.2} is R^{7.2}-substituted or unsubstituted C₁-C₄ alkyl. In embodiments, R^{6.2} is R^{7.2}-substituted or unsubstituted C₁-C₃ alkyl. In embodiments, R^{6.2} is R^{7.2}-substituted C₁-C₆ alkyl. In embodiments, R^{6.2} is R^{7.2}-substituted C₁-C₅ alkyl. In embodiments, R^{6.2} is R^{7.2}-substituted C₁-C₄ alkyl. In embodiments, R^{6.2} is R^{7.2}-substituted C₁-C₃ alkyl. In embodiments, R^{6.2} is unsubstituted C₁-C₆ alkyl. In embodiments, R^{6.2} is unsubstituted C₁-C₅ alkyl. In embodiments, R^{6.2} is unsubstituted C₁-C₄ alkyl. In embodiments, R^{6.2} is unsubstituted C₁-C₃ alkyl. In embodiments, R^{6.2} is unsubstituted methyl.

R⁷, R^{7.1} and R^{7.2} are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. R⁷, R^{7.1} and R^{7.2} may be independently unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

In embodiments, the compound of Formula (I) or the compound of Formula (II) is a compound of Formula (III). The compound of Formula (III) is also known as CPI-444 and has the following structure:

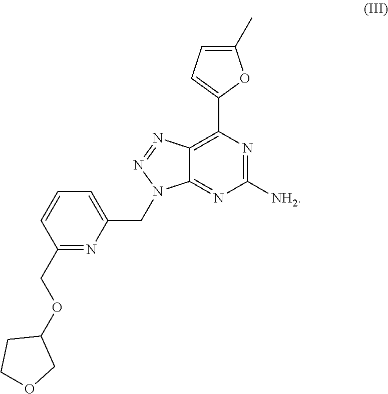 [SMILES: Cc1ccc(-c2nc(N)nc3c2nnn3Cc2cccc(COC3CCOC3)n2)o1]

In embodiments, the compound of Formula (III) is a compound of Formula (IIIA), which has the following structure:

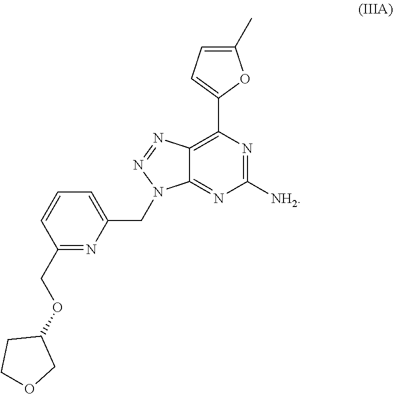

In embodiments, the compound of Formula (III) is a compound of Formula (IIIB), which has the following structure:

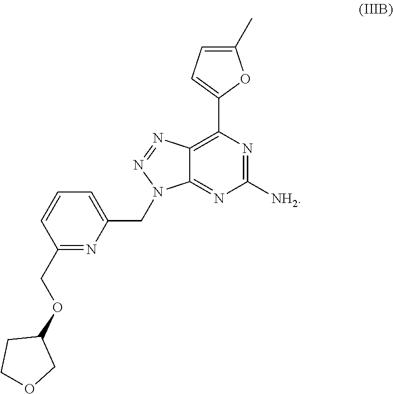

Elevated Levels of Adenosine A2A Receptor Gene Expression

The methods provided herein are particularly useful for the treatment of cancer in subjects who have: (i) an elevated level of adenosine A2A receptors relative to a control; (ii) an elevated level of adenosine A2A receptors relative to a control and an elevated level of CD73 relative to a control; (iii) an elevated level of adenosine A2A receptors relative to a control, an elevated level of CD73 relative to a control, and an elevated level of PD-L1 relative to a control; and (iv) an elevated level of adenosine A2A receptors relative to a control, and an elevated level of PD-L1 relative to a control.

Adenosine A2A receptor levels may be detected at either the protein or gene expression level. Proteins expressed by adenosine A2A receptors can be quantified by immunohistochemistry (IHC) or flow cytometry with an antibody that detects the proteins. Adenosine A2A receptor expression can be quantified by multiple platforms such as real-time polymerase chain reaction (rtPCR), Nanostring, RNAseq, or in situ hybridization. There is a range of adenosine A2A receptor expression across as measured by Nanostring. One skilled in the art will understand the importance of selecting a threshold of adenosine A2A receptor expression that constitutes elevated levels of adenosine A2A receptors. Controls are also valuable for determining the significance of data. For example, if values for a given parameter are widely variant in controls, variation in test samples will not be considered as significant. In some examples of the disclosed methods, when the expression level of adenosine A2A receptor genes is assessed, the adenosine A2A receptor level is compared with a control expression level of adenosine A2A receptor genes. By control expression level is meant the expression level of adenosine A2A receptors from a sample or subject lacking cancer, a sample or subject at a selected stage of cancer or cancer state, or in the absence of a particular variable such as a therapeutic agent. Alternatively, the control level comprises a known amount of adenosine A2A receptor genes. Such a known amount correlates with an average level of subjects lacking cancer, at a selected stage of cancer or cancer state, or in the absence of a particular variable such as a therapeutic agent. A control level also includes the expression level of adenosine A2A receptor genes from one or more selected samples or subjects as described herein. For example, a control level includes an assessment of the expression level of adenosine A2A receptor genes in a sample from a subject that does not have cancer, is at a selected stage of cancer or cancer state, or have cancer but have not yet received treatment for the cancer. Another exemplary control level includes an assessment of the expression level of adenosine A2A receptor genes in samples taken from multiple subjects that do not have cancer, are at a selected stage of cancer, or have cancer but have not yet received treatment for the cancer. In embodiments, the control is multiple subjects who have cancer and who are anti-PD-L1 resistant or anti-PD-L1 refractory. In some embodiments, a threshold for elevated adenosine A2A receptor levels is above the median expression level of a group of control sample, where the control sample is optionally a group of subjects who have cancer and are anti-PD-L1 resistant or anti-PD-L1 refractory. In some embodiments it is above the first quartile of adenosine A2A receptor gene expression in a group of control sample, where the control sample is optionally a group of subjects who have cancer and are anti-PD-L1 resistant or anti-PD-L1 refractory. In some embodiments it is above the third quartile of adenosine A2A receptor gene expression in a group of control sample, where the control sample is optionally a group of subjects who have cancer and are anti-PD-L1 resistant or anti-PD-L1 refractory. In some embodiments it is above the 5th percentile of adenosine A2A receptor gene expression in a group of control sample, where the control sample is optionally a group of subjects who have cancer and are anti-PD-L1 resistant or anti-PD-L1 refractory. In some embodiments it is above the 10th percentile of adenosine A2A receptor gene expression in a group of control sample, where the control sample is optionally a group of subjects who have cancer and are anti-PD-L1 resistant or anti-PD-L1 refractory. In some embodiments it is above the 20th percentile of adenosine A2A receptor gene expression in a group of control sample, where the control sample is optionally a group of subjects who have cancer and are anti-PD-L1 resistant or anti-PD-L1 refractory. In some embodiments it is above the 30th percentile of adenosine A2A receptor gene expression in a group of control sample, where the control sample is optionally a group of subjects who have cancer and are anti-PD-L1 resistant or anti-PD-L1 refractory. In some embodiments it is above the 40th percentile of adenosine A2A receptor gene expression in a group of control sample, where the control sample is optionally a group of subjects who have cancer and are anti-PD-L1 resistant or anti-PD-L1 refractory. In some embodiments it is above the 45th percentile of adenosine A2A receptor gene expression in a group of control sample, where the control sample is optionally a group of subjects who have cancer and are anti-PD-L1 resistant or anti-PD-L1 refractory. In some embodiments it is above the 50th percentile of adenosine A2A receptor gene expression in a group of control sample, where the control sample is optionally a group of subjects who have cancer and are anti-PD-L1 resistant or anti-PD-L1 refractory. In some embodiments it is above the 60th percentile of adenosine A2A receptor gene expression in a group of control sample, where the control sample is optionally a group of subjects who have cancer and are anti-PD-L1 resistant or anti-PD-L1 refractory. In some embodiments it is above the 70th percentile of adenosine A2A receptor gene expression in a group of control sample, where the control sample is optionally a group of subjects who have cancer and are anti-PD-L1 resistant or anti-PD-L1 refractory. In some embodiments it is above the 80th percentile of adenosine A2A receptor gene expression in a group of control sample, where the control sample is optionally a group of subjects who have cancer and are anti-PD-L1 resistant or anti-PD-L1 refractory. In some embodiments it is above the 90th percentile of adenosine A2A receptor gene expression in a group of control sample, where the control sample is optionally a group of subjects who have cancer and are anti-PD-L1 resistant or anti-PD-L1 refractory. In embodiments, the control sample is from a cancer tumor of a group of subjects who are anti-PD-L1 resistant or anti-PD-L1 refractory.

In embodiments, quantitative rtPCR, Nanostring, RNAseq, and in situ hybridization are platforms to quantitate adenosine A2A receptor gene expression. For Nanostring, RNA is extracted from tumor samples and a known quantity of RNA is placed on the Nanostring machine for gene expression detection using gene specific probes. The number of counts of adenosine A2A receptors within a sample is determined and normalized to a set of housekeeping genes. To determine a threshold for elevated adenosine A2A receptor levels, one skilled in the art could assess adenosine levels in a control group of samples (e.g., tumor samples from cancer subjects who are anti-PD-L1 resistant or anti-PD-L1 refractory) and select the 10^{th}, 20^{th}, 25^{th} 30^{th}, 40^{th}, 50^{th}, 60^{th}, 70^{th}, 75^{th}, 80^{th} or 90^{th} percentile of adenosine A2A receptor gene expression. In embodiments, the 10th percentile of adensoinse A2A receptor gene expression is selected as the threshold for elevated adenosine A2A receptor levels. In embodiments, the 20th percentile of adensoinse A2A receptor gene expression is selected as the threshold for elevated adenosine A2A receptor levels. In embodiments, the 25th percentile of adensoinse A2A receptor gene expression is selected as the threshold for elevated adenosine A2A receptor levels. In embodiments, the 30th percentile of adensoinse A2A receptor gene expression is selected as the threshold for elevated adenosine A2A receptor levels. In embodiments, the 40th percentile of adensoinse A2A receptor gene expression is selected as the threshold for elevated adenosine A2A receptor levels. In embodiments, the 50th percentile of adensoinse A2A receptor gene expression is selected as the threshold for elevated adenosine A2A receptor levels. In embodiments, the 60th percentile of adensoinse A2A receptor gene expression is selected as the threshold for elevated adenosine A2A receptor levels. In embodiments, the 70th percentile of adensoinse A2A receptor gene expression is selected as the threshold for elevated adenosine A2A receptor levels. In embodiments, the 75th percentile of adensoinse A2A receptor gene expression is selected as the threshold for elevated adenosine A2A receptor levels. In embodiments, the 80th percentile of adensoinse A2A receptor gene expression is selected as the threshold for elevated adenosine A2A receptor levels. In embodiments, the 90th percentile of adensoinse A2A receptor gene expression is selected as the threshold for elevated adenosine A2A receptor levels.

The elevated level of adenosine A2A receptors may be determined using standard methods commonly known in the art. For example, the elevated level of adenosine A2A receptors may be calculated by determining the percentage of cells that are positive for adenosine A2A receptors cells. The cells may be tumor cells, tumor infiltrating cells, stromal cells, vasculature cells, or a composite thereof. In embodiments, the cells are tumor cells. The “percentage of cells that are positive for adenosine A2A receptors” can also be referred to as the elevated level of adenosine A2A receptors. In embodiments, the percentage of cells that are positive for adenosine A2A receptors is greater than or equal to 1%. In embodiments, the percentage of cells that are positive for adenosine A2A receptors is greater than or equal to 2%. In embodiments, the percentage of cells that are positive for adenosine A2A receptors is greater than or equal to 3%. In embodiments, the percentage of cells that are positive for adenosine A2A receptors is greater than or equal to 4%. In embodiments, the percentage of cells that are positive for adenosine A2A receptors is greater than or equal to 5%. In embodiments, the percentage of cells that are positive for adenosine A2A receptors is greater than or equal to 6%. In embodiments, the percentage of cells that are positive for adenosine A2A receptors is greater than or equal to 7%. In embodiments, the percentage of cells that are positive for adenosine A2A receptors is greater than or equal to 8%. In embodiments, the percentage of cells that are positive for adenosine A2A receptors is greater than or equal to 9%. In embodiments, the percentage of cells that are positive for adenosine A2A receptors is greater than or equal to 10%. In embodiments, the percentage of cells that are positive for adenosine A2A receptors is greater than or equal to 11%. In embodiments, the percentage of cells that are positive for adenosine A2A receptors is greater than or equal to 12%. In embodiments, the percentage of cells that are positive for adenosine A2A receptors is greater than or equal to 13%. In embodiments, the percentage of cells that are positive for adenosine A2A receptors is greater than or equal to 14%. In embodiments, the percentage of cells that are positive for adenosine A2A receptors is greater than or equal to 15%. In embodiments, the percentage of cells that are positive for adenosine A2A receptors is greater than or equal to 16%. In embodiments, the percentage of cells that are positive for adenosine A2A receptors is greater than or equal to 17%. In embodiments, the percentage of cells that are positive for adenosine A2A receptors is greater than or equal to 18%. In embodiments, the percentage of cells that are positive for adenosine A2A receptors is greater than or equal to 19%. In embodiments, the percentage of cells that are positive for adenosine A2A receptors is greater than or equal to 20%. In embodiments, the percentage of cells that are positive for adenosine A2A receptors is greater than or equal to 21%. In embodiments, the percentage of cells that are positive for adenosine A2A receptors is greater than or equal to 22%. In embodiments, the percentage of cells that are positive for adenosine A2A receptors is greater than or equal to 23%. In embodiments, the percentage of cells that are positive for adenosine A2A receptors is greater than or equal to 24%. In embodiments, the percentage of cells that are positive for adenosine A2A receptors is greater than or equal to 25%. In embodiments, the percentage of cells that are positive for adenosine A2A receptors is greater than or equal to 26%. In embodiments, the percentage of cells that are positive for adenosine A2A receptors is greater than or equal to 27%. In embodiments, the percentage of cells that are positive for adenosine A2A receptors is greater than or equal to 28%. In embodiments, the percentage of cells that are positive for adenosine A2A receptors is greater than or equal to 29%. In embodiments, the percentage of cells that are positive for adenosine A2A receptors is greater than or equal to 30%. In embodiments, the percentage of cells that are positive for adenosine A2A receptors is greater than or equal to 31%. In embodiments, the percentage of cells that are positive for adenosine A2A receptors is greater than or equal to 32%. In embodiments, the percentage of cells that are positive for adenosine A2A receptors is greater than or equal to 33%. In embodiments, the percentage of cells that are positive for adenosine A2A receptors is greater than or equal to 34%. In embodiments, the percentage of cells that are positive for adenosine A2A receptors is greater than or equal to 35%. In embodiments, the percentage of cells that are positive for adenosine A2A receptors is greater than or equal to 36%. In embodiments, the percentage of cells that are positive for adenosine A2A receptors is greater than or equal to 37%. In embodiments, the percentage of cells that are positive for adenosine A2A receptors is greater than or equal to 38%. In embodiments, the percentage of cells that are positive for adenosine A2A receptors is greater than or equal to 39%. In embodiments, the percentage of cells that are positive for adenosine A2A receptors is greater than or equal to 40%. In embodiments, the percentage of cells that are positive for adenosine A2A receptors is greater than or equal to 41%. In embodiments, the percentage of cells that are positive for adenosine A2A receptors is greater than or equal to 42%. In embodiments, the percentage of cells that are positive for adenosine A2A receptors is greater than or equal to 43%. In embodiments, the percentage of cells that are positive for adenosine A2A receptors is greater than or equal to 44%. In embodiments, the percentage of cells that are positive for adenosine A2A receptors is greater than or equal to 45%. In embodiments, the percentage of cells that are positive for adenosine A2A receptors is greater than or equal to 46%. In embodiments, the percentage of cells that are positive for adenosine A2A receptors is greater than or equal to 47%. In embodiments, the percentage of cells that are positive for adenosine A2A receptors is greater than or equal to 48%. In embodiments, the percentage of cells that are positive for adenosine A2A receptors is greater than or equal to 49%. In embodiments, the percentage of cells that are positive for adenosine A2A receptors is greater than or equal to 50%. In embodiments, the percentage of cells that are positive for adenosine A2A receptors is greater than or equal to 51%. In embodiments, the percentage of cells that are positive for adenosine A2A receptors is greater than or equal to 52%. In embodiments, the percentage of cells that are positive for adenosine A2A receptors is greater than or equal to 53%. In embodiments, the percentage of cells that are positive for adenosine A2A receptors is greater than or equal to 54%. In embodiments, the percentage of cells that are positive for adenosine A2A receptors is greater than or equal to 55%. In embodiments, the percentage of cells that are positive for adenosine A2A receptors is greater than or equal to 56%. In embodiments, the percentage of cells that are positive for adenosine A2A receptors is greater than or equal to 57%. In embodiments, the percentage of cells that are positive for adenosine A2A receptors is greater than or equal to 58%. In embodiments, the percentage of cells that are positive for adenosine A2A receptors is greater than or equal to 59%. In embodiments, the percentage of cells that are positive for adenosine A2A receptors is greater than or equal to 60%. In embodiments, the percentage of cells that are positive for adenosine A2A receptors is greater than or equal to 61%. In embodiments, the percentage of cells that are positive for adenosine A2A receptors is greater than or equal to 62%. In embodiments, the percentage of cells that are positive for adenosine A2A receptors is greater than or equal to 63%. In embodiments, the percentage of cells that are positive for adenosine A2A receptors is greater than or equal to 64%. In embodiments, the percentage of cells that are positive for adenosine A2A receptors is greater than or equal to 65%. In embodiments, the percentage of cells that are positive for adenosine A2A receptors is greater than or equal to 66%. In embodiments, the percentage of cells that are positive for adenosine A2A receptors is greater than or equal to 67%. In embodiments, the percentage of cells that are positive for adenosine A2A receptors is greater than or equal to 68%. In embodiments, the percentage of cells that are positive for adenosine A2A receptors is greater than or equal to 69%. In embodiments, the percentage of cells that are positive for adenosine A2A receptors is greater than or equal to 70%. In embodiments, the percentage of cells that are positive for adenosine A2A receptors is greater than or equal to 71%. In embodiments, the percentage of cells that are positive for adenosine A2A receptors is greater than or equal to 72%. In embodiments, the percentage of cells that are positive for adenosine A2A receptors is greater than or equal to 73%. In embodiments, the percentage of cells that are positive for adenosine A2A receptors is greater than or equal to 74%. In embodiments, the percentage of cells that are positive for adenosine A2A receptors is greater than or equal to 75%. In embodiments, the percentage of cells that are positive for adenosine A2A receptors is greater than or equal to 76%. In embodiments, the percentage of cells that are positive for adenosine A2A receptors is greater than or equal to 77%. In embodiments, the percentage of cells that are positive for adenosine A2A receptors is greater than or equal to 78%. In embodiments, the percentage of cells that are positive for adenosine A2A receptors is greater than or equal to 79%. In embodiments, the percentage of cells that are positive for adenosine A2A receptors is greater than or equal to 80%. In embodiments, the percentage of cells that are positive for adenosine A2A receptors is greater than or equal to 81%. In embodiments, the percentage of cells that are positive for adenosine A2A receptors is greater than or equal to 82%. In embodiments, the percentage of cells that are positive for adenosine A2A receptors is greater than or equal to 83%. In embodiments, the percentage of cells that are positive for adenosine A2A receptors is greater than or equal to 84%. In embodiments, the percentage of cells that are positive for adenosine A2A receptors is greater than or equal to 85%. In embodiments, the percentage of cells that are positive for adenosine A2A receptors is greater than or equal to 86%. In embodiments, the percentage of cells that are positive for adenosine A2A receptors is greater than or equal to 87%. In embodiments, the percentage of cells that are positive for adenosine A2A receptors is greater than or equal to 88%. In embodiments, the percentage of cells that are positive for adenosine A2A receptors is greater than or equal to 89%. In embodiments, the percentage of cells that are positive for adenosine A2A receptors is greater than or equal to 90%. In embodiments, the percentage of cells that are positive for adenosine A2A receptors is greater than or equal to 91%. In embodiments, the percentage of cells that are positive for adenosine A2A receptors is greater than or equal to 92%. In embodiments, the percentage of cells that are positive for adenosine A2A receptors is greater than or equal to 93%. In embodiments, the percentage of cells that are positive for adenosine A2A receptors is greater than or equal to 94%. In embodiments, the percentage of cells that are positive for adenosine A2A receptors is greater than or equal to 95%. In embodiments, the percentage of cells that are positive for adenosine A2A receptors is greater than or equal to 96%. In embodiments, the percentage of cells that are positive for adenosine A2A receptors is greater than or equal to 97%. In embodiments, the percentage of cells that are positive for adenosine A2A receptors is greater than or equal to 98%. In embodiments, the percentage of cells that are positive for adenosine A2A receptors is greater than or equal to 99%. Any of the embodiments described herein for the percentage of cells that are positive for adenosine A2A receptors can be considered an elevated level of adenosine A2A receptors.

In embodiments, the elevated level of adenosine A2A receptors may be determined by calculating the H-score for the elevated level of adenosine A2A receptors. The H-score may be calculated for membrane adenosine A2A receptors or cytosolic adenosine A2A receptors. The H score may be calculated for tumor cells. Thus, the elevated level of adenosine A2A receptors may have an H-score. As used herein, an “H-score” or “Histoscore” is a numerical value determined by a semi-quantitative method commonly known for immunohistochemically evaluating protein expression in tumor samples. The H-score may be calculated using the following formula: [1×(% cells 1+)+2×(% cells 2+)+3×(% cells 3+)].

According to this formula, the H-score is calculated by determining the percentage of cells having a given staining intensity level (i.e., level 1+, 2+, or 3+ from lowest to highest intensity level), weighting the percentage of cells having the given intensity level by multiplying the cell percentage by a factor (e.g., 1, 2, or 3) that gives more relative weight to cells with higher-intensity membrane staining, and summing the results to obtain a H-score. Commonly H-scores range from 0 to 300. Further description on the determination of H-scores in tumor cells can be found in Hirsch F R, Varella-Garcia M, Bunn P A Jr., et al. (Epidermal growth factor receptor in non-small-cell lung carcinomas: Correlations between gene copy number and protein expression and impact on prognosis. J Clin Oncol 21: 3798-3807, 2003) and John T, Liu G, Tsao M-S(Overview of molecular testing in non-small-cell lung cancer: Mutational analysis, gene copy number, protein expression and other biomarkers of EGFR for the prediction of response to tyrosine kinase inhibitors. Oncogene 28:S14-S23, 2009), which are hereby incorporated by reference in their entirety and for all purposes. Immunohistochemistry or other methods known in the art may be used for detecting adenosine A2A receptors expression. In embodiments, the H-score of a cancer cell is determined. In embodiments, the H-score of a non-cancer cell is determined. In embodiments, the non-cancer cell is a stromal cell. In embodiments, the H-score of a cancer cell and a non-cancer cell is determined.

In embodiments, the elevated level of adenosine A2A receptors has an H-score of at least 1 (e.g., 5, 10, 20, 40, 50, 60, 70, 80, 90, 100, 110, 120, 130, 140, 150, 155, 160, 165, 170, 175, 180, 185, 190, 195, 200, 205, 210, 215, 220, 230, 240, 250, 260, 270, 280, 290, 300). In embodiments, the elevated level of adenosine A2A receptors has an H-score of at least 1. In embodiments, the elevated level of adenosine A2A receptors has an H-score of at least 5. In embodiments, the elevated level of adenosine A2A receptors has an H-score of at least 10. In embodiments, the elevated level of adenosine A2A receptors has an H-score of at least 15. In embodiments, the elevated level of adenosine A2A receptors has an H-score of at least 20. In embodiments, the elevated level of adenosine A2A receptors has an H-score of at least 25. In embodiments, the elevated level of adenosine A2A receptors has an H-score of at least 30. In embodiments, the elevated level of adenosine A2A receptors has an H-score of at least 35. In embodiments, the elevated level of adenosine A2A receptors has an H-score of at least 40. In embodiments, the elevated level of adenosine A2A receptors has an H-score of at least 45. In embodiments, the elevated level of adenosine A2A receptors has an H-score of at least 50. In embodiments, the elevated level of adenosine A2A receptors has an H-score of at least 55. In embodiments, the elevated level of adenosine A2A receptors has an H-score of at least 60. In embodiments, the elevated level of adenosine A2A receptors has an H-score of at least 65. In embodiments, the elevated level of adenosine A2A receptors has an H-score of at least 70. In embodiments, the elevated level of adenosine A2A receptors has an H-score of at least 75. In embodiments, the elevated level of adenosine A2A receptors has an H-score of at least 80. In embodiments, the elevated level of adenosine A2A receptors has an H-score of at least 85. In embodiments, the elevated level of adenosine A2A receptors has an H-score of at least 90. In embodiments, the elevated level of adenosine A2A receptors has an H-score of at least 95. In embodiments, the elevated level of adenosine A2A receptors has an H-score of at least 100. In embodiments, the elevated level of adenosine A2A receptors has an H-score of at least 105. In embodiments, the elevated level of adenosine A2A receptors has an H-score of at least 110. In embodiments, the elevated level of adenosine A2A receptors has an H-score of at least 115. In embodiments, the elevated level of adenosine A2A receptors has an H-score of at least 120. In embodiments, the elevated level of adenosine A2A receptors has an H-score of at least 125. In embodiments, the elevated level of adenosine A2A receptors has an H-score of at least 130. In embodiments, the elevated level of adenosine A2A receptors has an H-score of at least 135. In embodiments, the elevated level of adenosine A2A receptors has an H-score of at least 140. In embodiments, the elevated level of adenosine A2A receptors has an H-score of at least 145. In embodiments, the elevated level of adenosine A2A receptors has an H-score of at least 150. In embodiments, the elevated level of adenosine A2A receptors has an H-score of at least 155. In embodiments, the elevated level of adenosine A2A receptors has an H-score of at least 160. In embodiments, the elevated level of adenosine A2A receptors has an H-score of at least 165. In embodiments, the elevated level of adenosine A2A receptors has an H-score of at least 170. In embodiments, the elevated level of adenosine A2A receptors has an H-score of at least 175. In embodiments, the elevated level of adenosine A2A receptors has an H-score of at least 180. In embodiments, the elevated level of adenosine A2A receptors has an H-score of at least 185. In embodiments, the elevated level of adenosine A2A receptors has an H-score of at least 190. In embodiments, the elevated level of adenosine A2A receptors has an H-score of at least 195. In embodiments, the elevated level of adenosine A2A receptors has an H-score of at least 200. In embodiments, the elevated level of adenosine A2A receptors has an H-score of at least 205. In embodiments, the elevated level of adenosine A2A receptors has an H-score of at least 210. In embodiments, the elevated level of adenosine A2A receptors has an H-score of at least 215. In embodiments, the elevated level of adenosine A2A receptors has an H-score of at least 220. In embodiments, the elevated level of adenosine A2A receptors has an H-score of at least 230. In embodiments, the elevated level of adenosine A2A receptors has an H-score of at least 240. In embodiments, the elevated level of adenosine A2A receptors has an H-score of at least 250. In embodiments, the elevated level of adenosine A2A receptors has an H-score of at least 260. In embodiments, the elevated level of adenosine A2A receptors has an H-score of at least 270. In embodiments, the elevated level of adenosine A2A receptors has an H-score of at least 280. In embodiments, the elevated level of adenosine A2A receptors has an H-score of at least 290. In embodiments, the elevated level of adenosine A2A receptors has an H-score of 300.

In embodiments, the elevated level of adenosine A2A receptors has an H-score of about 1. In embodiments, the elevated level of adenosine A2A receptors has an H-score of about 5. In embodiments, the elevated level of adenosine A2A receptors has an H-score of about 10. In embodiments, the elevated level of adenosine A2A receptors has an H-score of about 15. In embodiments, the elevated level of adenosine A2A receptors has an H-score of about 20. In embodiments, the elevated level of adenosine A2A receptors has an H-score of about 25. In embodiments, the elevated level of adenosine A2A receptors has an H-score of about 30. In embodiments, the elevated level of adenosine A2A receptors has an H-score of about 35. In embodiments, the elevated level of adenosine A2A receptors has an H-score of about 40. In embodiments, the elevated level of adenosine A2A receptors has an H-score of about 45. In embodiments, the elevated level of adenosine A2A receptors has an H-score of about 50. In embodiments, the elevated level of adenosine A2A receptors has an H-score of about 55. In embodiments, the elevated level of adenosine A2A receptors has an H-score of about 60. In embodiments, the elevated level of adenosine A2A receptors has an H-score of about 65. In embodiments, the elevated level of adenosine A2A receptors has an H-score of about 70. In embodiments, the elevated level of adenosine A2A receptors has an H-score of about 75. In embodiments, the elevated level of adenosine A2A receptors has an H-score of about 80. In embodiments, the elevated level of adenosine A2A receptors has an H-score of about 85. In embodiments, the elevated level of adenosine A2A receptors has an H-score of about 90. In embodiments, the elevated level of adenosine A2A receptors has an H-score of about 95. In embodiments, the elevated level of adenosine A2A receptors has an H-score of about 100. In embodiments, the elevated level of adenosine A2A receptors has an H-score of about 105. In embodiments, the elevated level of adenosine A2A receptors has an H-score of about 110. In embodiments, the elevated level of adenosine A2A receptors has an H-score of about 115. In embodiments, the elevated level of adenosine A2A receptors has an H-score of about 120. In embodiments, the elevated level of adenosine A2A receptors has an H-score of about 125. In embodiments, the elevated level of adenosine A2A receptors has an H-score of about 130. In embodiments, the elevated level of adenosine A2A receptors has an H-score of about 135. In embodiments, the elevated level of adenosine A2A receptors has an H-score of about 140. In embodiments, the elevated level of adenosine A2A receptors has an H-score of about 145. In embodiments, the elevated level of adenosine A2A receptors has an H-score of about 150. In embodiments, the elevated level of adenosine A2A receptors has an H-score of about 155. In embodiments, the elevated level of adenosine A2A receptors has an H-score of about 160. In embodiments, the elevated level of adenosine A2A receptors has an H-score of about 165. In embodiments, the elevated level of adenosine A2A receptors has an H-score of about 170. In embodiments, the elevated level of adenosine A2A receptors has an H-score of about 175. In embodiments, the elevated level of adenosine A2A receptors has an H-score of about 180. In embodiments, the elevated level of adenosine A2A receptors has an H-score of about 185. In embodiments, the elevated level of adenosine A2A receptors has an H-score of about 190. In embodiments, the elevated level of adenosine A2A receptors has an H-score of about 195. In embodiments, the elevated level of adenosine A2A receptors has an H-score of about 200. In embodiments, the elevated level of adenosine A2A receptors has an H-score of about 205. In embodiments, the elevated level of adenosine A2A receptors has an H-score of about 210. In embodiments, the elevated level of adenosine A2A receptors has an H-score of about 215. In embodiments, the elevated level of adenosine A2A receptors has an H-score of about 220. In embodiments, the elevated level of adenosine A2A receptors has an H-score of about 230. In embodiments, the elevated level of adenosine A2A receptors has an H-score of about 240. In embodiments, the elevated level of adenosine A2A receptors has an H-score of about 250. In embodiments, the elevated level of adenosine A2A receptors has an H-score of about 260. In embodiments, the elevated level of adenosine A2A receptors has an H-score of about 270. In embodiments, the elevated level of adenosine A2A receptors has an H-score of about 280. In embodiments, the elevated level of adenosine A2A receptors has an H-score of about 290. In embodiments, the elevated level of adenosine A2A receptors has an H-score of 300. In embodiments, the elevated level of adenosine A2A receptors has an H-score of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299 or 300.

Elevated Levels of CD73

The methods provided herein are useful for the treatment of cancer in subjects who have an elevated level of adenosine A2A receptor relative to a control. In embodiments, the methods provided herein are useful for the treatment of cancer in subjects who have an elevated level of adenosine A2A receptor relative to a control, and an elevated level of CD73 relative to a control. In embodiments, the methods provided herein are useful for the treatment of cancer in subjects who have an elevated level of CD73 relative to a control.

CD73 levels may be detected at either the protein or gene expression level. CD73 protein can be quantified by immunohistochemistry (IHC) or flow cytometry with an antibody that detects CD73. CD73 gene expression can be quantified by multiple platforms such as real-time polymerase chain reaction (rtPCR), Nanostring, or in situ hybridization. There is a range of CD73 expression across and within tumor types that shows concordance when measured with either IHC or by Nanostring. One skilled in the art will understand the importance of selecting a threshold of CD73 expression that constitutes elevated levels. Controls are also valuable for determining the significance of data. For example, if values for a given parameter are widely variant in controls, variation in test samples will not be considered as significant. In some examples of the disclosed methods, when the expression level of CD73 is assessed, the level is compared with a control expression level of CD73. By control expression level is meant the expression level of CD73 from a sample or subject lacking cancer, a sample or subject at a selected stage of cancer or cancer state, or in the absence of a particular variable such as a therapeutic agent. Alternatively, the control level comprises a known amount of CD73. Such a known amount correlates with an average level of subjects lacking cancer, at a selected stage of cancer or cancer state, or in the absence of a particular variable such as a therapeutic agent. A control level also includes the expression level of CD73 from one or more selected samples or subjects as described herein. For example, a control level includes an assessment of the expression level of CD73 in a sample from a subject that does not have cancer, is at a selected stage of cancer or cancer state, or have cancer but have not yet received treatment for the cancer. Another exemplary control level includes an assessment of the expression level of CD73 in samples taken from multiple subjects that do not have cancer, are at a selected stage of cancer, or have cancer but have not yet received treatment for the cancer. In embodiments, a threshold for elevated CD73 may be above the median expression level of a group of control samples, optionally wherein the control is tumor samples from cancer subjects who are anti-PD-L1 resistant or anti-PD-L1 refractory. In embodiments it is above the first quartile of CD73 expression in a group of control samples, optionally wherein the control is tumor samples from cancer subjects who are anti-PD-L1 resistant or anti-PD-L1 refractory. In embodiments it is above the third quartile of CD73 expression in a group of control samples, optionally wherein the control is tumor samples from cancer subjects who are anti-PD-L1 resistant or anti-PD-L1 refractory. In some embodiments it is above the 10th percentile of CD73 expression for a group of control samples, optionally wherein the control is tumor samples from cancer subjects who are anti-PD-L1 resistant or anti-PD-L1 refractory. In some embodiments it is above the 20th percentile of CD73 expression for a group of control samples, optionally wherein the control is tumor samples from cancer subjects who are anti-PD-L1 resistant or anti-PD-L1 refractory. In some embodiments it is above the 25th percentile of CD73 expression for a group of control samples, optionally wherein the control is tumor samples from cancer subjects who are anti-PD-L1 resistant or anti-PD-L1 refractory. In some embodiments it is above the 30th percentile of CD73 expression for a group of control samples, optionally wherein the control is tumor samples from cancer subjects who are anti-PD-L1 resistant or anti-PD-L1 refractory. In some embodiments it is above the 40th percentile of CD73 expression for a group of control samples, optionally wherein the control is tumor samples from cancer subjects who are anti-PD-L1 resistant or anti-PD-L1 refractory. In some embodiments it is above the 50th percentile of CD73 expression for a group of control samples, optionally wherein the control is tumor samples from cancer subjects who are anti-PD-L1 resistant or anti-PD-L1 refractory. In some embodiments it is above the 60th percentile of CD73 expression for a group of control samples, optionally wherein the control is tumor samples from cancer subjects who are anti-PD-L1 resistant or anti-PD-L1 refractory. In some embodiments it is above the 70th percentile of CD73 expression for a group of control samples, optionally wherein the control is tumor samples from cancer subjects who are anti-PD-L1 resistant or anti-PD-L1 refractory. In some embodiments it is above the 75th percentile of CD73 expression for a group of control samples, optionally wherein the control is tumor samples from cancer subjects who are anti-PD-L1 resistant or anti-PD-L1 refractory. In some embodiments it is above the 80th percentile of CD73 expression for a group of control samples, optionally wherein the control is tumor samples from cancer subjects who are anti-PD-L1 resistant or anti-PD-L1 refractory. In some embodiments it is above the 90th percentile of CD73 expression for a group of control samples, optionally wherein the control is tumor samples from cancer subjects who are anti-PD-L1 resistant or anti-PD-L1 refractory.

When the control level includes the expression level of CD73 in a sample or subject in the absence of a therapeutic agent, the control sample or subject is optionally the same sample or subject to be tested before or after treatment with a therapeutic agent or is a selected sample or subject in the absence of the therapeutic agent. Alternatively, a control level is an average expression level calculated from a number of subjects without a particular disease. A control level also includes a known control level or value known in the art.

The elevated level of CD73 may be determined using standard methods commonly known in the art. For example, the elevated level of CD73 may be calculated by determining the percentage of cells that are positive for CD73 cells. The cells may be tumor cells, tumor infiltrating cells, stromal cells, vasculature cells, or a composite thereof. In embodiments, the cells are tumor cells. In embodiments, the percentage of cells that are positive for CD73 is greater than or equal to 1%. In embodiments, the percentage of cells that are positive for CD73 is greater than or equal to 5%. In embodiments, the percentage of cells that are positive for CD73 is greater than or equal to 10%. In embodiments, the percentage of cells that are positive for CD73 is greater than or equal to 15%. In embodiments, the percentage of cells that are positive for CD73 is greater than or equal to 20%. In embodiments, the percentage of cells that are positive for CD73 is greater than or equal to 25%. In embodiments, the percentage of cells that are positive for CD73 is greater than or equal to 30%. In embodiments, the percentage of cells that are positive for CD73 is greater than or equal to 35%. In embodiments, the percentage of cells that are positive for CD73 is greater than or equal to 40%. In embodiments, the percentage of cells that are positive for CD73 is greater than or equal to 45%. In embodiments, the percentage of cells that are positive for CD73 is greater than or equal to 50%. In embodiments, the percentage of cells that are positive for CD73 is greater than or equal to 55%. In embodiments, the percentage of cells that are positive for CD73 is greater than or equal to 60%. In embodiments, the percentage of cells that are positive for CD73 is greater than or equal to 65%. In embodiments, the percentage of cells that are positive for CD73 is greater than or equal to 70%. In embodiments, the percentage of cells that are positive for CD73 is greater than or equal to 75%. In embodiments, the percentage of cells that are positive for CD73 is greater than or equal to 80%. In embodiments, the percentage of cells that are positive for CD73 is greater than or equal to 85%. In embodiments, the percentage of cells that are positive for CD73 is greater than or equal to 90%. In embodiments, the percentage of cells that are positive for CD73 is greater than or equal to 95%. Any of the embodiments described herein for the percentage of cells that are positive for CD73 can be considered an elevated level of CD73.

In embodiments, the elevated level of CD73 may be determined by calculating the H-score for the elevated level of CD73. The H-score may be calculated for membrane CD73 or cytosolic CD73. The H score may be calculated for tumor cells. Thus, the elevated level of CD73 may have an H-score. As used herein, an “H-score” or “Histoscore” is a numerical value determined by a semi-quantitative method commonly known for immunohistochemically evaluating protein expression in tumor samples. The H-score may be calculated using the following formula: [1×(% cells 1+)+2×(% cells 2+)+3×(% cells 3+)].

According to this formula, the H-score is calculated by determining the percentage of cells having a given staining intensity level (i.e., level 1+, 2+, or 3+ from lowest to highest intensity level), weighting the percentage of cells having the given intensity level by multiplying the cell percentage by a factor (e.g., 1, 2, or 3) that gives more relative weight to cells with higher-intensity membrane staining, and summing the results to obtain a H-score. Commonly H-scores range from 0 to 300. Further description on the determination of H-scores in tumor cells can be found in Hirsch F R, Varella-Garcia M, Bunn P A Jr., et al. (Epidermal growth factor receptor in non-small-cell lung carcinomas: Correlations between gene copy number and protein expression and impact on prognosis. J Clin Oncol 21: 3798-3807, 2003) and John T, Liu G, Tsao M-S(Overview of molecular testing in non-small-cell lung cancer: Mutational analysis, gene copy number, protein expression and other biomarkers of EGFR for the prediction of response to tyrosine kinase inhibitors. Oncogene 28:S14-S23, 2009), which are hereby incorporated by reference in their entirety and for all purposes. Immunohistochemistry or other methods known in the art may be used for detecting CD73 expression. In embodiments, the H-score of a cancer cell is determined. In embodiments, the H-score of a non-cancer cell is determined. In embodiments, the non-cancer cell is a stromal cell. In embodiments, the H-score of a cancer cell and a non-cancer cell is determined.

In embodiments, the elevated level of CD73 has an H-score of at least 1 (e.g., 5, 10, 20, 40, 50, 60, 70, 80, 90, 100, 110, 120, 130, 140, 150, 155, 160, 165, 170, 175, 180, 185, 190, 195, 200, 205, 210, 215, 220, 230, 240, 250, 260, 270, 280, 290, 300). In embodiments, the elevated level of CD73 has an H-score of at least 1. In embodiments, the elevated level of CD73 has an H-score of at least 5. In embodiments, the elevated level of CD73 has an H-score of at least 10. In embodiments, the elevated level of CD73 has an H-score of at least 15. In embodiments, the elevated level of CD73 has an H-score of at least 20. In embodiments, the elevated level of CD73 has an H-score of at least 25. In embodiments, the elevated level of CD73 has an H-score of at least 30. In embodiments, the elevated level of CD73 has an H-score of at least 35. In embodiments, the elevated level of CD73 has an H-score of at least 40. In embodiments, the elevated level of CD73 has an H-score of at least 45. In embodiments, the elevated level of CD73 has an H-score of at least 50. In embodiments, the elevated level of CD73 has an H-score of at least 55. In embodiments, the elevated level of CD73 has an H-score of at least 60. In embodiments, the elevated level of CD73 has an H-score of at least 65. In embodiments, the elevated level of CD73 has an H-score of at least 70. In embodiments, the elevated level of CD73 has an H-score of at least 75. In embodiments, the elevated level of CD73 has an H-score of at least 80. In embodiments, the elevated level of CD73 has an H-score of at least 85. In embodiments, the elevated level of CD73 has an H-score of at least 90. In embodiments, the elevated level of CD73 has an H-score of at least 95. In embodiments, the elevated level of CD73 has an H-score of at least 100. In embodiments, the elevated level of CD73 has an H-score of at least 105. In embodiments, the elevated level of CD73 has an H-score of at least 110. In embodiments, the elevated level of CD73 has an H-score of at least 115. In embodiments, the elevated level of CD73 has an H-score of at least 120. In embodiments, the elevated level of CD73 has an H-score of at least 125. In embodiments, the elevated level of CD73 has an H-score of at least 130. In embodiments, the elevated level of CD73 has an H-score of at least 135. In embodiments, the elevated level of CD73 has an H-score of at least 140. In embodiments, the elevated level of CD73 has an H-score of at least 145. In embodiments, the elevated level of CD73 has an H-score of at least 150. In embodiments, the elevated level of CD73 has an H-score of at least 155. In embodiments, the elevated level of CD73 has an H-score of at least 160. In embodiments, the elevated level of CD73 has an H-score of at least 165. In embodiments, the elevated level of CD73 has an H-score of at least 170. In embodiments, the elevated level of CD73 has an H-score of at least 175. In embodiments, the elevated level of CD73 has an H-score of at least 180. In embodiments, the elevated level of CD73 has an H-score of at least 185. In embodiments, the elevated level of CD73 has an H-score of at least 190. In embodiments, the elevated level of CD73 has an H-score of at least 195. In embodiments, the elevated level of CD73 has an H-score of at least 200. In embodiments, the elevated level of CD73 has an H-score of at least 205. In embodiments, the elevated level of CD73 has an H-score of at least 210. In embodiments, the elevated level of CD73 has an H-score of at least 215. In embodiments, the elevated level of CD73 has an H-score of at least 220. In embodiments, the elevated level of CD73 has an H-score of at least 230. In embodiments, the elevated level of CD73 has an H-score of at least 240. In embodiments, the elevated level of CD73 has an H-score of at least 250. In embodiments, the elevated level of CD73 has an H-score of at least 260. In embodiments, the elevated level of CD73 has an H-score of at least 270. In embodiments, the elevated level of CD73 has an H-score of at least 280. In embodiments, the elevated level of CD73 has an H-score of at least 290. In embodiments, the elevated level of CD73 has an H-score of 300.

In embodiments, the elevated level of CD73 has an H-score of about 1. In embodiments, the elevated level of CD73 has an H-score of about 5. In embodiments, the elevated level of CD73 has an H-score of about 10. In embodiments, the elevated level of CD73 has an H-score of about 15. In embodiments, the elevated level of CD73 has an H-score of about 20. In embodiments, the elevated level of CD73 has an H-score of about 25. In embodiments, the elevated level of CD73 has an H-score of about 30. In embodiments, the elevated level of CD73 has an H-score of about 35. In embodiments, the elevated level of CD73 has an H-score of about 40. In embodiments, the elevated level of CD73 has an H-score of about 45. In embodiments, the elevated level of CD73 has an H-score of about 50. In embodiments, the elevated level of CD73 has an H-score of about 55. In embodiments, the elevated level of CD73 has an H-score of about 60. In embodiments, the elevated level of CD73 has an H-score of about 65. In embodiments, the elevated level of CD73 has an H-score of about 70. In embodiments, the elevated level of CD73 has an H-score of about 75. In embodiments, the elevated level of CD73 has an H-score of about 80. In embodiments, the elevated level of CD73 has an H-score of about 85. In embodiments, the elevated level of CD73 has an H-score of about 90. In embodiments, the elevated level of CD73 has an H-score of about 95. In embodiments, the elevated level of CD73 has an H-score of about 100. In embodiments, the elevated level of CD73 has an H-score of about 105. In embodiments, the elevated level of CD73 has an H-score of about 110. In embodiments, the elevated level of CD73 has an H-score of about 115. In embodiments, the elevated level of CD73 has an H-score of about 120. In embodiments, the elevated level of CD73 has an H-score of about 125. In embodiments, the elevated level of CD73 has an H-score of about 130. In embodiments, the elevated level of CD73 has an H-score of about 135. In embodiments, the elevated level of CD73 has an H-score of about 140. In embodiments, the elevated level of CD73 has an H-score of about 145. In embodiments, the elevated level of CD73 has an H-score of about 150. In embodiments, the elevated level of CD73 has an H-score of about 155. In embodiments, the elevated level of CD73 has an H-score of about 160. In embodiments, the elevated level of CD73 has an H-score of about 165. In embodiments, the elevated level of CD73 has an H-score of about 170. In embodiments, the elevated level of CD73 has an H-score of about 175. In embodiments, the elevated level of CD73 has an H-score of about 180. In embodiments, the elevated level of CD73 has an H-score of about 185. In embodiments, the elevated level of CD73 has an H-score of about 190. In embodiments, the elevated level of CD73 has an H-score of about 195. In embodiments, the elevated level of CD73 has an H-score of about 200. In embodiments, the elevated level of CD73 has an H-score of about 205. In embodiments, the elevated level of CD73 has an H-score of about 210. In embodiments, the elevated level of CD73 has an H-score of about 215. In embodiments, the elevated level of CD73 has an H-score of about 220. In embodiments, the elevated level of CD73 has an H-score of about 230. In embodiments, the elevated level of CD73 has an H-score of about 240. In embodiments, the elevated level of CD73 has an H-score of about 250. In embodiments, the elevated level of CD73 has an H-score of about 260. In embodiments, the elevated level of CD73 has an H-score of about 270. In embodiments, the elevated level of CD73 has an H-score of about 280. In embodiments, the elevated level of CD73 has an H-score of about 290. In embodiments, the elevated level of CD73 has an H-score of 300. In embodiments, the elevated level of CD73 has an H-score of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299 or 300.

The elevated level of CD73 may be determined by determining stromal score for the elevated level of CD73. A “stromal score” as used herein, refers to the percentage of CD73 expressing stromal cells (e.g., non-tumor cells including, for example, fibroblasts, pericytes, endothelial cells, etc.) per tumor surface in a tissue sample. Immunohistochemistry or other methods known in the art may be used for detecting CD73 expression on stromal cells.

In embodiments, the elevated level of CD73 has a stromal score of at least 50% (e.g., 51, 52, 53, 54, 55, 56, 57, 58, 59, 60, 65, 70, 75, 80, 85, 90, 95, 100%). In embodiments, the elevated level of CD73 has a stromal score of at least 51%. In embodiments, the elevated level of CD73 has a stromal score of at least 52%. In embodiments, the elevated level of CD73 has a stromal score of at least 53%. In embodiments, the elevated level of CD73 has a stromal score of at least 54%. In embodiments, the elevated level of CD73 has a stromal score of at least 55%. In embodiments, the elevated level of CD73 has a stromal score of at least 56%. In embodiments, the elevated level of CD73 has a stromal score of at least 57%. In embodiments, the elevated level of CD73 has a stromal score of at least 58%. In embodiments, the elevated level of CD73 has a stromal score of at least 59%. In embodiments, the elevated level of CD73 has a stromal score of at least 60%. In embodiments, the elevated level of CD73 has a stromal score of at least 65%. In embodiments, the elevated level of CD73 has a stromal score of at least 70%. In embodiments, the elevated level of CD73 has a stromal score of at least 75%. In embodiments, the elevated level of CD73 has a stromal score of at least 80%. In embodiments, the elevated level of CD73 has a stromal score of at least 85%. In embodiments, the elevated level of CD73 has a stromal score of at least 90%. In embodiments, the elevated level of CD73 has a stromal score of at least 95%. In embodiments, the elevated level of CD73 has a stromal score of 100%.

In embodiments, CD73 gene expression is used to assay for elevated CD73. For example, quantitative rtPCR, Nanostring, and in situ hybridization are platforms to quantitate gene expression. For Nanostring, RNA is extracted from tumor samples and a known quantity of RNA is placed on the Nanostring machine for gene expression detection using gene specific probes. The number of counts of CD73 within a samples is determined and normalized to a set of “housekeeping” genes. Nanostring “housekeeping” genes include: ABCF1, AGK, ALAS1, AMMECR1L, CC2D1B, CNOT10, CNOT4, COG7, DDX50, DHX16, DNAJC14, EDC3, EIFB4, ERCC3, FCF1, G6PD, GPATCH3, GUSB, HDAC3, HPRT1, MRPS5, MTMR14, NOL7, NUBP1, POLR2A, PPIA, PRPF38A, SAP130, SDHA, SF3A3, TBP, TLK2, TMUB2, TRIM39, TUBB, USP39, ZC3H14, ZKSCAN5, ZNF143, ZNF346. Across the biopsy samples assessed in the clinical trial data included herein, the 33rd percentile of CD73 expression was 125.5 counts and was used to identify patients with elevated CD73. The normalized count level of CD73 may be 100, 150, 200, 250, 300, 350, 400, 450 or 500 to determine elevated CD73.

In embodiments, quantitative rtPCR is used to quantitate the amount of CD73 RNA within a sample. Known amounts of a synthetic template of CD73 may be used to empirically derive a standard curve to compare CD73 levels with the number of thermocycles required to detect CD73. To determine a threshold for elevated CD73, one skilled in the art could assess CD73 levels in a control group of samples (e.g., tumors from cancer subjects who are anti-PD-L1 resistant or anti-PD-L1 refractory) and select the 10th percentile of CD73 expression. In embodiments, the 20th percentile of CD73 expression is selected as a threshold for elevated CD73. In embodiments, the 25th percentile of CD73 expression is selected as a threshold for elevated CD73. In embodiments, the 30th percentile of CD73 expression is selected as a threshold for elevated CD73. In embodiments, the 40th percentile of CD73 expression is selected as a threshold for elevated CD73. In embodiments, the 50th percentile of CD73 expression is selected as a threshold for elevated CD73. In embodiments, the 60th percentile of CD73 expression is selected as a threshold for elevated CD73. In embodiments, the 70th percentile of CD73 expression is selected as a threshold for elevated CD73. In embodiments, the 75th percentile of CD73 expression is selected as a threshold for elevated CD73. In embodiments, the 80th percentile of CD73 expression is selected as a threshold for elevated CD73. In embodiments, the 90th percentile of CD73 expression is selected as a threshold for elevated CD73. Alternatively, one skilled in the art could utilize a threshold for CD73 determined by IHC or Nanostring, and quantify the amount of CD73 present at that threshold using quantitative rtPCR.

In embodiments, the CD73 antigen forms part of a cell. In embodiments, the cell is a tumor cell. In embodiments, the cell is a cancer cell. In embodiments, the cell is a non-cancer cell. In embodiments, the cell is an immune cell. In embodiments, the cell is a stromal cell (e.g., non-tumor cells including, for example, fibroblasts, pericytes, endothelial cells, etc.). In embodiments, the cell is a T cell. In embodiments, the CD73 antigen forms part of a tumor cell and not a stromal cell. In embodiments, the CD73 antigen forms part of a stromal cell and not a tumor cell. In embodiments, the CD73 antigen forms part of a stromal cell and a tumor cell.

Elevated Levels of PD-L1

The methods provided herein are particularly useful for the treatment of cancer in subjects who have: (i) an elevated level of adenosine A2A receptors relative to a control, and an elevated level of PD-L1 relative to a control; and (i) an elevated level of adenosine A2A receptors relative to a control, an elevated level of CD73 relative to a control, and an elevated level of PD-L1 relative to a control.

PD-L1 levels may be detected at either the protein or gene expression level. Proteins expressed by PD-L1 can be quantified by immunohistochemistry (IHC) or flow cytometry with an antibody that detects the proteins. PD-L1 expression can be quantified by multiple platforms such as real-time polymerase chain reaction (rtPCR), Nanostring, or in situ hybridization. There is a range of PD-L1 expression across and within tumor types that shows concordance when measured with either IHC or by Nanostring. One skilled in the art will understand the importance of selecting a threshold of PD-L1 expression that constitutes elevated levels of PD-L1. Controls are also valuable for determining the significance of data. For example, if values for a given parameter are widely variant in controls, variation in test samples will not be considered as significant. In some examples of the disclosed methods, when the expression level of PD-L1 genes is assessed, the PD-L1 level is compared with a control expression level of PD-L1 genes. By control expression level is meant the expression level of PD-L1 from a sample or subject lacking cancer, a sample or subject at a selected stage of cancer or cancer state, or in the absence of a particular variable such as a therapeutic agent. Alternatively, the control level comprises a known amount of PD-L1 genes. Such a known amount correlates with an average level of subjects lacking cancer, at a selected stage of cancer or cancer state, or in the absence of a particular variable such as a therapeutic agent. A control level also includes the expression level of PD-L1 genes from one or more selected samples or subjects as described herein. For example, a control level includes an assessment of the expression level of PD-L1 genes in a sample from a subject that does not have cancer, is at a selected stage of cancer or cancer state, or have cancer but have not yet received treatment for the cancer. Another exemplary control level includes an assessment of the expression level of PD-L1 genes in samples taken from multiple subjects that do not have cancer, are at a selected stage of cancer, or have cancer but have not yet received treatment for the cancer. In embodiments, the control is multiple subjects who have cancer and who are anti-PD-L1 resistant or anti-PD-L1 refractory. In some embodiments, a threshold for elevated PD-L1 levels may be above the median expression level of a group of control sample, where the control sample is optionally a group of subjects who have cancer and are anti-PD-L1 resistant or anti-PD-L1 refractory. In some embodiments it may be above the first quartile of PD-L1 gene expression in a group of control sample, where the control sample is optionally a group of subjects who have cancer and are anti-PD-L1 resistant or anti-PD-L1 refractory. In some embodiments it may be above the third quartile of PD-L1 gene expression in a group of control sample, where the control sample is optionally a group of subjects who have cancer and are anti-PD-L1 resistant or anti-PD-L1 refractory. In some embodiments it may be above the 5th percentile of PD-L1 gene expression in a group of control sample, where the control sample is optionally a group of subjects who have cancer and are anti-PD-L1 resistant or anti-PD-L1 refractory. In some embodiments it may be above the 10th percentile of PD-L1 gene expression in a group of control sample, where the control sample is optionally a group of subjects who have cancer and are anti-PD-L1 resistant or anti-PD-L1 refractory. In some embodiments it may be above the 20th percentile of PD-L1 gene expression in a group of control sample, where the control sample is optionally a group of subjects who have cancer and are anti-PD-L1 resistant or anti-PD-L1 refractory. In some embodiments it may be above the 30th percentile of PD-L1 gene expression in a group of control sample, where the control sample is optionally a group of subjects who have cancer and are anti-PD-L1 resistant or anti-PD-L1 refractory. In some embodiments it may be above the 40th percentile of PD-L1 gene expression in a group of control sample, where the control sample is optionally a group of subjects who have cancer and are anti-PD-L1 resistant or anti-PD-L1 refractory. In some embodiments it may be above the 45th percentile of PD-L1 gene expression in a group of control sample, where the control sample is optionally a group of subjects who have cancer and are anti-PD-L1 resistant or anti-PD-L1 refractory. In some embodiments it may be above the 50th percentile of PD-L1 gene expression in a group of control sample, where the control sample is optionally a group of subjects who have cancer and are anti-PD-L1 resistant or anti-PD-L1 refractory. In some embodiments it may be above the 60th percentile of PD-L1 gene expression in a group of control sample, where the control sample is optionally a group of subjects who have cancer and are anti-PD-L1 resistant or anti-PD-L1 refractory. In some embodiments it may be above the 70th percentile of PD-L1 gene expression in a group of control sample, where the control sample is optionally a group of subjects who have cancer and are anti-PD-L1 resistant or anti-PD-L1 refractory. In some embodiments it may be above the 80th percentile of PD-L1 gene expression in a group of control sample, where the control sample is optionally a group of subjects who have cancer and are anti-PD-L1 resistant or anti-PD-L1 refractory. In some embodiments it may be above the 90th percentile of PD-L1 gene expression in a group of control sample, where the control sample is optionally a group of subjects who have cancer and are anti-PD-L1 resistant or anti-PD-L1 refractory. In embodiments, the control sample is from a cancer tumor of a group of subjects who are anti-PD-L1 resistant or anti-PD-L1 refractory.

The level of PD-L1 (e.g., the absolute level of PD-L1) is measured on immune cells and/or tumor cells. In embodiments, the level of PD-L1 (e.g., the absolute level of PD-L1) is measured on immune cells and/or tumor cells is measured by immunohistochemistry. In embodiments, the cancer tumor comprises an elevated level of PD-L1 relative to a control. In embodiments, the control is a negative control. In embodiments, the elevated level is an increase of at least about 1%, 5%, 10%, 15%, 20%, 25%, 30%, 35%, 40%, 45%, 50%, 55%, 60%, 65%, 70%, 75%, 80%, 85%, 90%, 100%, 2-fold, 5-fold, 10-fold, 20-fold, or 100-fold compared to the control. In embodiments, the elevated level is an increase of at least about 10% compared to the control (e.g., cancer tumor in subjects who are anti-PD-L1 resistant or anti-PD-L1 refractory). In embodiments, the elevated level is an increase of at least about 25% compared to the control (e.g., cancer tumor in subjects who are anti-PD-L1 resistant or anti-PD-L1 refractory). In embodiments, the elevated level is an increase of at least about 50% compared to the control (e.g., cancer tumor in subjects who are anti-PD-L1 resistant or anti-PD-L1 refractory). In embodiments, the elevated level is an increase of at least about 75% compared to the control (e.g., cancer tumor in subjects who are anti-PD-L1 resistant or anti-PD-L1 refractory). In embodiments, the cancer tumor comprises an elevated level of PD-L1 in all tumor cells (e.g., in the tumor cell population taken as a whole) relative to a control. In embodiments, the increased level of PD-L1 is an increased level of PD-L1 mRNA (i.e., mRNA that encodes PD-L1). In embodiments, the increased level of PD-L1 is an increased level of PD-L1 protein. In embodiments, the level of PD-L1 is measured by immunohistochemistry. In embodiments, the level of PD-L1 (e.g., as assessed by immunohistochemistry) is given a value between 0% and 100%. In embodiments, the level of PD-L1 is compared to a negative control. In embodiments, the level of PD-L1 expression is measured by gene expression. In embodiments, the level of PD-L1 is given a z-score. In embodiments, detecting PD-L1 expression comprises use of the Fluidigm real-time PCR platform. In embodiments, a PD-L1 expression score for a tumor sample of interest is calculated as the arithmetic mean of normalized mRNA or protein expression levels, in the tumor sample, for PD-L1. In embodiments, the PD-L1 expression score is the geometric mean of expression values (e.g., normalized mRNA or protein expression levels) for PD-L1. In embodiments, the geometric mean is calculated by multiplying numbers together and then take a square root (for two numbers), cube root (for three numbers) etc. (calculable as the n th root of a product of n numbers). Non-limiting examples of methods for detecting an increase in the level of PD-L1 mRNA included qRT-PCR, microarray hybridization methods, and RNA sequencing (RNAseq). Non-limiting examples of methods for detecting an increase in the level of PD-L1 protein include High-performance liquid chromatography (HPLC), Liquid chromatography-mass spectrometry (LC/MS), Enzyme-linked immunosorbent assay (ELISA), immunoelectrophoresis, Western blot, radioimmuno assays, and protein immunostaining (e.g., immunohistochemistry).

The elevated level of PD-L1 is determined using standard methods commonly known in the art. For example, the elevated level of PD-L1 is calculated by determining the percentage of cells that are positive for PD-L1 cells. The cells is tumor cells, tumor infiltrating cells, stromal cells, vasculature cells, or a composite thereof. In embodiments, the cells are tumor cells. The percentage of cells that are positive for PD-L1 can also be referred to as the elevated level of PD-L1. In embodiments, the percentage of cells that are positive for PD-L1 is greater than or equal to 1%. In embodiments, the percentage of cells that are positive for PD-L1 is greater than or equal to 5%. In embodiments, the percentage of cells that are positive for PD-L1 is greater than or equal to 10%. In embodiments, the percentage of cells that are positive for PD-L1 is greater than or equal to 15%. In embodiments, the percentage of cells that are positive for PD-L1 is greater than or equal to 20%. In embodiments, the percentage of cells that are positive for PD-L1 is greater than or equal to 25%. In embodiments, the percentage of cells that are positive for PD-L1 is greater than or equal to 30%. In embodiments, the percentage of cells that are positive for PD-L1 is greater than or equal to 35%. In embodiments, the percentage of cells that are positive for PD-L1 is greater than or equal to 40%. In embodiments, the percentage of cells that are positive for PD-L1 is greater than or equal to 45%. In embodiments, the percentage of cells that are positive for PD-L1 is greater than or equal to 50%. In embodiments, the percentage of cells that are positive for PD-L1 is greater than or equal to 55%. In embodiments, the percentage of cells that are positive for PD-L1 is greater than or equal to 60%. In embodiments, the percentage of cells that are positive for PD-L1 is greater than or equal to 65%. In embodiments, the percentage of cells that are positive for PD-L1 is greater than or equal to 70%. In embodiments, the percentage of cells that are positive for PD-L1 is greater than or equal to 75%. In embodiments, the percentage of cells that are positive for PD-L1 is greater than or equal to 80%. In embodiments, the percentage of cells that are positive for PD-L1 is greater than or equal to 85%. In embodiments, the percentage of cells that are positive for PD-L1 is greater than or equal to 90%. In embodiments, the percentage of cells that are positive for PD-L1 is greater than or equal to 95%. Any of the embodiments described herein for the percentage of cells that are positive for PD-L1 can be considered an elevated level of PD-L1.

In embodiments, the elevated level of PD-L1 is determined by calculating the H-score for the elevated level of PD-L1. The H-score is calculated for membrane PD-L1 or cytosolic PD-L1. The H score is calculated for tumor cells. Thus, the elevated level of PD-L1 may have an H-score. As used herein, an “H-score” or “Histoscore” is a numerical value determined by a semi-quantitative method commonly known for immunohistochemically evaluating protein expression in tumor samples. The H-score is calculated using the following formula: [1×(% cells 1+)+2×(% cells 2+)+3×(% cells 3+)].

According to this formula, the H-score is calculated by determining the percentage of cells having a given staining intensity level (i.e., level 1+, 2+, or 3+ from lowest to highest intensity level), weighting the percentage of cells having the given intensity level by multiplying the cell percentage by a factor (e.g., 1, 2, or 3) that gives more relative weight to cells with higher-intensity membrane staining, and summing the results to obtain a H-score. Commonly H-scores range from 0 to 300. Further description on the determination of H-scores in tumor cells can be found in Hirsch F R, Varella-Garcia M, Bunn P A Jr., et al. (Epidermal growth factor receptor in non-small-cell lung carcinomas: Correlations between gene copy number and protein expression and impact on prognosis. J Clin Oncol 21: 3798-3807, 2003) and John T, Liu G, Tsao M-S(Overview of molecular testing in non-small-cell lung cancer: Mutational analysis, gene copy number, protein expression and other biomarkers of EGFR for the prediction of response to tyrosine kinase inhibitors. Oncogene 28:S14-S23, 2009), which are hereby incorporated by reference in their entirety and for all purposes. Immunohistochemistry or other methods known in the art is used for detecting PD-L1 expression. In embodiments, the H-score of a cancer cell is determined. In embodiments, the H-score of a non-cancer cell is determined. In embodiments, the non-cancer cell is a stromal cell. In embodiments, the H-score of a cancer cell and a non-cancer cell is determined.

In embodiments, the elevated level of PD-L1 has an H-score of at least 1 (e.g., 5, 10, 20, 40, 50, 60, 70, 80, 90, 100, 110, 120, 130, 140, 150, 155, 160, 165, 170, 175, 180, 185, 190, 195, 200, 205, 210, 215, 220, 230, 240, 250, 260, 270, 280, 290, 300). In embodiments, the elevated level of PD-L1 has an H-score of at least 1. In embodiments, the elevated level of PD-L1 has an H-score of at least 5. In embodiments, the elevated level of PD-L1 has an H-score of at least 10. In embodiments, the elevated level of PD-L1 has an H-score of at least 15. In embodiments, the elevated level of PD-L1 has an H-score of at least 20. In embodiments, the elevated level of PD-L1 has an H-score of at least 25. In embodiments, the elevated level of PD-L1 has an H-score of at least 30. In embodiments, the elevated level of PD-L1 has an H-score of at least 35. In embodiments, the elevated level of PD-L1 has an H-score of at least 40. In embodiments, the elevated level of PD-L1 has an H-score of at least 45. In embodiments, the elevated level of PD-L1 has an H-score of at least 50. In embodiments, the elevated level of PD-L1 has an H-score of at least 55. In embodiments, the elevated level of PD-L1 has an H-score of at least 60. In embodiments, the elevated level of PD-L1 has an H-score of at least 65. In embodiments, the elevated level of PD-L1 has an H-score of at least 70. In embodiments, the elevated level of PD-L1 has an H-score of at least 75. In embodiments, the elevated level of PD-L1 has an H-score of at least 80. In embodiments, the elevated level of PD-L1 has an H-score of at least 85. In embodiments, the elevated level of PD-L1 has an H-score of at least 90. In embodiments, the elevated level of PD-L1 has an H-score of at least 95. In embodiments, the elevated level of PD-L1 has an H-score of at least 100. In embodiments, the elevated level of PD-L1 has an H-score of at least 105. In embodiments, the elevated level of PD-L1 has an H-score of at least 110. In embodiments, the elevated level of PD-L1 has an H-score of at least 115. In embodiments, the elevated level of PD-L1 has an H-score of at least 120. In embodiments, the elevated level of PD-L1 has an H-score of at least 125. In embodiments, the elevated level of PD-L1 has an H-score of at least 130. In embodiments, the elevated level of PD-L1 has an H-score of at least 135. In embodiments, the elevated level of PD-L1 has an H-score of at least 140. In embodiments, the elevated level of PD-L1 has an H-score of at least 145. In embodiments, the elevated level of PD-L1 has an H-score of at least 150. In embodiments, the elevated level of PD-L1 has an H-score of at least 155. In embodiments, the elevated level of PD-L1 has an H-score of at least 160. In embodiments, the elevated level of PD-L1 has an H-score of at least 165. In embodiments, the elevated level of PD-L1 has an H-score of at least 170. In embodiments, the elevated level of PD-L1 has an H-score of at least 175. In embodiments, the elevated level of PD-L1 has an H-score of at least 180. In embodiments, the elevated level of PD-L1 has an H-score of at least 185. In embodiments, the elevated level of PD-L1 has an H-score of at least 190. In embodiments, the elevated level of PD-L1 has an H-score of at least 195. In embodiments, the elevated level of PD-L1 has an H-score of at least 200. In embodiments, the elevated level of PD-L1 has an H-score of at least 205. In embodiments, the elevated level of PD-L1 has an H-score of at least 210. In embodiments, the elevated level of PD-L1 has an H-score of at least 215. In embodiments, the elevated level of PD-L1 has an H-score of at least 220. In embodiments, the elevated level of PD-L1 has an H-score of at least 230. In embodiments, the elevated level of PD-L1 has an H-score of at least 240. In embodiments, the elevated level of PD-L1 has an H-score of at least 250. In embodiments, the elevated level of PD-L1 has an H-score of at least 260. In embodiments, the elevated level of PD-L1 has an H-score of at least 270. In embodiments, the elevated level of PD-L1 has an H-score of at least 280. In embodiments, the elevated level of PD-L1 has an H-score of at least 290. In embodiments, the elevated level of PD-L1 has an H-score of 300.

In embodiments, the elevated level of PD-L1 has an H-score of about 1. In embodiments, the elevated level of PD-L1 has an H-score of about 5. In embodiments, the elevated level of PD-L1 has an H-score of about 10. In embodiments, the elevated level of PD-L1 has an H-score of about 15. In embodiments, the elevated level of PD-L1 has an H-score of about 20. In embodiments, the elevated level of PD-L1 has an H-score of about 25. In embodiments, the elevated level of PD-L1 has an H-score of about 30. In embodiments, the elevated level of PD-L1 has an H-score of about 35. In embodiments, the elevated level of PD-L1 has an H-score of about 40. In embodiments, the elevated level of PD-L1 has an H-score of about 45. In embodiments, the elevated level of PD-L1 has an H-score of about 50. In embodiments, the elevated level of PD-L1 has an H-score of about 55. In embodiments, the elevated level of PD-L1 has an H-score of about 60. In embodiments, the elevated level of PD-L1 has an H-score of about 65. In embodiments, the elevated level of PD-L1 has an H-score of about 70. In embodiments, the elevated level of PD-L1 has an H-score of about 75. In embodiments, the elevated level of PD-L1 has an H-score of about 80. In embodiments, the elevated level of PD-L1 has an H-score of about 85. In embodiments, the elevated level of PD-L1 has an H-score of about 90. In embodiments, the elevated level of PD-L1 has an H-score of about 95. In embodiments, the elevated level of PD-L1 has an H-score of about 100. In embodiments, the elevated level of PD-L1 has an H-score of about 105. In embodiments, the elevated level of PD-L1 has an H-score of about 110. In embodiments, the elevated level of PD-L1 has an H-score of about 115. In embodiments, the elevated level of PD-L1 has an H-score of about 120. In embodiments, the elevated level of PD-L1 has an H-score of about 125. In embodiments, the elevated level of PD-L1 has an H-score of about 130. In embodiments, the elevated level of PD-L1 has an H-score of about 135. In embodiments, the elevated level of PD-L1 has an H-score of about 140. In embodiments, the elevated level of PD-L1 has an H-score of about 145. In embodiments, the elevated level of PD-L1 has an H-score of about 150. In embodiments, the elevated level of PD-L1 has an H-score of about 155. In embodiments, the elevated level of PD-L1 has an H-score of about 160. In embodiments, the elevated level of PD-L1 has an H-score of about 165. In embodiments, the elevated level of PD-L1 has an H-score of about 170. In embodiments, the elevated level of PD-L1 has an H-score of about 175. In embodiments, the elevated level of PD-L1 has an H-score of about 180. In embodiments, the elevated level of PD-L1 has an H-score of about 185. In embodiments, the elevated level of PD-L1 has an H-score of about 190. In embodiments, the elevated level of PD-L1 has an H-score of about 195. In embodiments, the elevated level of PD-L1 has an H-score of about 200. In embodiments, the elevated level of PD-L1 has an H-score of about 205. In embodiments, the elevated level of PD-L1 has an H-score of about 210. In embodiments, the elevated level of PD-L1 has an H-score of about 215. In embodiments, the elevated level of PD-L1 has an H-score of about 220. In embodiments, the elevated level of PD-L1 has an H-score of about 230. In embodiments, the elevated level of PD-L1 has an H-score of about 240. In embodiments, the elevated level of PD-L1 has an H-score of about 250. In embodiments, the elevated level of PD-L1 has an H-score of about 260. In embodiments, the elevated level of PD-L1 has an H-score of about 270. In embodiments, the elevated level of PD-L1 has an H-score of about 280. In embodiments, the elevated level of PD-L1 has an H-score of about 290. In embodiments, the elevated level of PD-L1 has an H-score of 300. In embodiments, the elevated level of PD-L1 has an H-score of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299 or 300.

Methods Using Adenosine Pathway Inhibitors

The disclosure provides methods of treating cancer in a subject in need thereof by administering a therapeutically effective amount of an adenosine pathway inhibitor to the subject to treat the cancer; wherein the subject has an elevated level of adenosine A2A receptors when compared to a control. In embodiments, the adenosine pathway inhibitor is an anti-CD73 compound. In embodiments, the adenosine pathway inhibitor is an anti-CD39 compound. In embodiments, the adenosine pathway inhibitor comprises an anti-CD73 compound and an anti-CD39 compound. In embodiments, the adenosine pathway inhibitor comprises an anti-CD73 compound and an adenosine A2A receptor antagonist. In embodiments, the adenosine pathway inhibitor comprises an adenosine A2A receptor antagonist and an anti-CD39 compound. In embodiments, the adenosine pathway inhibitor comprises an adenosine A2A receptor antagonist, an anti-CD73 compound, and an anti-CD39 compound. In embodiments, the adenosine pathway inhibitor is an adenosine A2A receptor antagonist. In embodiments, the adenosine A2A receptor antagonist is a compound of Formula (I) or a pharmaceutically acceptable salt thereof. In embodiments, the adenosine A2A receptor antagonist is a compound of Formula (II) or a pharmaceutically acceptable salt thereof. In embodiments, the adenosine A2A receptor antagonist is a compound of Formula (III) or a pharmaceutically acceptable salt thereof. In embodiments, the adenosine A2A receptor antagonist is a compound of Formula (IIIA) or a pharmaceutically acceptable salt thereof. In embodiments, the adenosine A2A receptor antagonist is a compound of Formula (IIIB) or a pharmaceutically acceptable salt thereof. In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the subject was responsive to prior PD-1 pathway inhibitor therapy. In embodiments, the subject is naïve to PD-1 pathway inhibitor therapy. In embodiments, the method of treating cancer is: (i) a method of increasing CD8-positive cells relative to the amount of regulatory T cells; (ii) a method of decreasing tumor volume; (iii) a method of enhancing anti-tumor immune memory; (iv) a method of treating a cancer tumor; or (v) two or more of the foregoing. In embodiments, the cancer is lung cancer. In embodiments, the lung cancer is non-small cell lung cancer. In embodiments, the cancer is melanoma. In embodiments, the melanoma is malignant melanoma. In embodiments, the cancer is breast cancer. In embodiments, the breast cancer is triple negative breast cancer. In embodiments, the cancer is colorectal cancer. In embodiments, the cancer is microsatellite instable colorectal cancer. In embodiments, the cancer is bladder cancer. In embodiments, the cancer is head and neck cancer. In embodiments, the cancer is renal cell cancer. In embodiments, the cancer is prostate cancer. In embodiments, the cancer is metastatic castration resistant prostate cancer. In embodiments, the methods of treating cancer in the subject further comprise administering a therapeutically effective amount of a chemotherapeutic agent.

The disclosure provides methods of treating cancer in a subject in need thereof by administering a therapeutically effective amount of a compound of Formula (III) to the subject to treat the cancer; wherein the subject has an elevated level of adenosine A2A receptors when compared to a control. In embodiments, the cancer is lung cancer, melanoma, breast cancer, colorectal cancer, bladder cancer, head and neck cancer, renal cell cancer, or prostate cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month. In embodiments, the methods of treating cancer in the subject further comprise administering a therapeutically effective amount of a chemotherapeutic agent.

The disclosure provides methods of treating lung cancer in a subject in need thereof by administering a therapeutically effective amount of a compound of Formula (III) to the subject to treat the lung cancer; wherein the subject has an elevated level of adenosine A2A receptors when compared to a control. In embodiments, the lung cancer is non-small cell lung cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating melanoma in a subject in need thereof by administering a therapeutically effective amount of a compound of Formula (III) to the subject to treat the melanoma; wherein the subject has an elevated level of adenosine A2A receptors when compared to a control. In embodiments, the melanoma is malignant melanoma. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating breast cancer in a subject in need thereof by administering a therapeutically effective amount of a compound of Formula (III) to the subject to treat the breast cancer; wherein the subject has an elevated level of adenosine A2A receptors when compared to a control. In embodiments, the breast cancer is triple negative breast cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating colorectal cancer in a subject in need thereof by administering a therapeutically effective amount of a compound of Formula (III) to the subject to treat the colorectal cancer; wherein the subject has an elevated level of adenosine A2A receptors when compared to a control. In embodiments, the colorectal cancer is microsatellite instable colorectal cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating bladder cancer in a subject in need thereof by administering a therapeutically effective amount of a compound of Formula (III) to the subject to treat the bladder cancer; wherein the subject has an elevated level of adenosine A2A receptors when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating head and neck cancer in a subject in need thereof by administering a therapeutically effective amount of a compound of Formula (III) to the subject to treat the head and neck cancer; wherein the subject has an elevated level of adenosine A2A receptors when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. n embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating renal cell cancer in a subject in need thereof by administering a therapeutically effective amount of a compound of Formula (III) to the subject to treat the renal cell cancer; wherein the subject has an elevated level of adenosine A2A receptors when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating prostate cancer in a subject in need thereof by administering a therapeutically effective amount of a compound of Formula (III) to the subject to treat the prostate cancer; wherein the subject has an elevated level of adenosine A2A receptors when compared to a control. In embodiments, the prostate cancer is castration-resistant prostate cancer or metastatic castration-resistant prostate cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating cancer in a subject in need thereof by administering a therapeutically effective amount of an adenosine pathway inhibitor to the subject to treat the cancer; wherein the subject has: (i) an elevated level of adenosine A2A receptors when compared to a control; and (ii) an elevated level of CD73 when compared to a control. In embodiments, the adenosine pathway inhibitor is an anti-CD73 compound. In embodiments, the adenosine pathway inhibitor is an anti-CD39 compound. In embodiments, the adenosine pathway inhibitor comprises an anti-CD73 compound and an anti-CD39 compound. In embodiments, the adenosine pathway inhibitor comprises an anti-CD73 compound and an adenosine A2A receptor antagonist. In embodiments, the adenosine pathway inhibitor comprises an adenosine A2A receptor antagonist and an anti-CD39 compound. In embodiments, the adenosine pathway inhibitor comprises an adenosine A2A receptor antagonist, an anti-CD73 compound, and an anti-CD39 compound. In embodiments, the adenosine pathway inhibitor is an adenosine A2A receptor antagonist. In embodiments, the adenosine A2A receptor antagonist is a compound of Formula (I) or a pharmaceutically acceptable salt thereof. In embodiments, the adenosine A2A receptor antagonist is a compound of Formula (II) or a pharmaceutically acceptable salt thereof. In embodiments, the adenosine A2A receptor antagonist is a compound of Formula (III) or a pharmaceutically acceptable salt thereof. In embodiments, the adenosine A2A receptor antagonist is a compound of Formula (IIIA) or a pharmaceutically acceptable salt thereof. In embodiments, the adenosine A2A receptor antagonist is a compound of Formula (IIIB) or a pharmaceutically acceptable salt thereof. In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the subject was responsive to prior PD-1 pathway inhibitor therapy. In embodiments, the subject is naïve to PD-1 pathway inhibitor therapy. In embodiments, the method of treating cancer is: (i) a method of increasing CD8-positive cells relative to the amount of regulatory T cells; (ii) a method of decreasing tumor volume; (iii) a method of enhancing anti-tumor immune memory; (iv) a method of treating a cancer tumor; or (v) two or more of the foregoing. In embodiments, the cancer is lung cancer. In embodiments, the lung cancer is non-small cell lung cancer. In embodiments, the cancer is melanoma. In embodiments, the melanoma is malignant melanoma. In embodiments, the cancer is breast cancer. In embodiments, the breast cancer is triple negative breast cancer. In embodiments, the cancer is colorectal cancer. In embodiments, the cancer is microsatellite instable colorectal cancer. In embodiments, the cancer is bladder cancer. In embodiments, the cancer is head and neck cancer. In embodiments, the cancer is renal cell cancer. In embodiments, the cancer is prostate cancer. In embodiments, the cancer is metastatic castration resistant prostate cancer. In embodiments, the methods of treating cancer in the subject further comprise administering a therapeutically effective amount of a chemotherapeutic agent.

The disclosure provides methods of treating cancer in a subject in need thereof by administering a therapeutically effective amount of a compound of Formula (III) to the subject to treat the cancer; wherein the subject has: (i) an elevated level of adenosine A2A receptors when compared to a control; and (ii) an elevated level of CD73 when compared to a control. In embodiments, the cancer is lung cancer, melanoma, breast cancer, colorectal cancer, bladder cancer, head and neck cancer, renal cell cancer, or prostate cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month. In embodiments, the methods of treating cancer in the subject further comprise administering a therapeutically effective amount of a chemotherapeutic agent.

The disclosure provides methods of treating lung cancer in a subject in need thereof by administering a therapeutically effective amount of a compound of Formula (III) to the subject to treat the lung cancer; wherein the subject has: (i) an elevated level of adenosine A2A receptors when compared to a control; and (ii) an elevated level of CD73 when compared to a control. In embodiments, the lung cancer is non-small cell lung cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating melanoma in a subject in need thereof by administering a therapeutically effective amount of a compound of Formula (III) to the subject to treat the melanoma; wherein the subject has: (i) an elevated level of adenosine A2A receptors when compared to a control; and (ii) an elevated level of CD73 when compared to a control. In embodiments, the melanoma is malignant melanoma. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating breast cancer in a subject in need thereof by administering a therapeutically effective amount of a compound of Formula (III) to the subject to treat the breast cancer; wherein the subject has: (i) an elevated level of adenosine A2A receptors when compared to a control; and (ii) an elevated level of CD73 when compared to a control. In embodiments, the breast cancer is triple negative breast cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating colorectal cancer in a subject in need thereof by administering a therapeutically effective amount of a compound of Formula (III) to the subject to treat the colorectal cancer; wherein the subject has: (i) an elevated level of adenosine A2A receptors when compared to a control; and (ii) an elevated level of CD73 when compared to a control. In embodiments, the colorectal cancer is microsatellite instable colorectal cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating bladder cancer in a subject in need thereof by administering a therapeutically effective amount of a compound of Formula (III) to the subject to treat the bladder cancer; wherein the subject has: (i) an elevated level of adenosine A2A receptors when compared to a control; and (ii) an elevated level of CD73 when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating head and neck cancer in a subject in need thereof by administering a therapeutically effective amount of a compound of Formula (III) to the subject to treat the head and neck cancer; wherein the subject has: (i) an elevated level of adenosine A2A receptors when compared to a control; and (ii) an elevated level of CD73 when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating renal cell cancer in a subject in need thereof by administering a therapeutically effective amount of a compound of Formula (III) to the subject to treat the renal cell cancer; wherein the subject has: (i) an elevated level of adenosine A2A receptors when compared to a control; and (ii) an elevated level of CD73 when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating prostate cancer in a subject in need thereof by administering a therapeutically effective amount of a compound of Formula (III) to the subject to treat the prostate cancer; wherein the subject has: (i) an elevated level of adenosine A2A receptors when compared to a control; and (ii) an elevated level of CD73 when compared to a control. In embodiments, the prostate cancer is castration-resistant prostate cancer or metastatic castration-resistant prostate cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating cancer in a subject in need thereof by administering a therapeutically effective amount of an adenosine pathway inhibitor to the subject to treat the cancer; wherein the subject has: (i) an elevated level of adenosine A2A receptors when compared to a control; (ii) an elevated level of CD73 when compared to a control; and (iii) an elevated level of PD-L1 when compared to a control. In embodiments, the adenosine pathway inhibitor is an anti-CD73 compound. In embodiments, the adenosine pathway inhibitor is an anti-CD39 compound. In embodiments, the adenosine pathway inhibitor comprises an anti-CD73 compound and an anti-CD39 compound. In embodiments, the adenosine pathway inhibitor comprises an anti-CD73 compound and an adenosine A2A receptor antagonist. In embodiments, the adenosine pathway inhibitor comprises an adenosine A2A receptor antagonist and an anti-CD39 compound. In embodiments, the adenosine pathway inhibitor comprises an adenosine A2A receptor antagonist, an anti-CD73 compound, and an anti-CD39 compound. In embodiments, the adenosine pathway inhibitor is an adenosine A2A receptor antagonist. In embodiments, the adenosine A2A receptor antagonist is a compound of Formula (I) or a pharmaceutically acceptable salt thereof. In embodiments, the adenosine A2A receptor antagonist is a compound of Formula (II) or a pharmaceutically acceptable salt thereof. In embodiments, the adenosine A2A receptor antagonist is a compound of Formula (III) or a pharmaceutically acceptable salt thereof. In embodiments, the adenosine A2A receptor antagonist is a compound of Formula (IIIA) or a pharmaceutically acceptable salt thereof. In embodiments, the adenosine A2A receptor antagonist is a compound of Formula (IIIB) or a pharmaceutically acceptable salt thereof. In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the subject was responsive to prior PD-1 pathway inhibitor therapy. In embodiments, the subject is naïve to PD-1 pathway inhibitor therapy. In embodiments, the method of treating cancer is: (i) a method of increasing CD8-positive cells relative to the amount of regulatory T cells; (ii) a method of decreasing tumor volume; (iii) a method of enhancing anti-tumor immune memory; (iv) a method of treating a cancer tumor; or (v) two or more of the foregoing. In embodiments, the cancer is lung cancer. In embodiments, the lung cancer is non-small cell lung cancer. In embodiments, the cancer is melanoma. In embodiments, the melanoma is malignant melanoma. In embodiments, the cancer is breast cancer. In embodiments, the breast cancer is triple negative breast cancer. In embodiments, the cancer is colorectal cancer. In embodiments, the cancer is microsatellite instable colorectal cancer. In embodiments, the cancer is bladder cancer. In embodiments, the cancer is head and neck cancer. In embodiments, the cancer is renal cell cancer. In embodiments, the cancer is prostate cancer. In embodiments, the cancer is metastatic castration resistant prostate cancer. In embodiments, the methods of treating cancer in the subject further comprise administering a therapeutically effective amount of a chemotherapeutic agent.

The disclosure provides methods of treating cancer in a subject in need thereof by administering a therapeutically effective amount of a compound of Formula (III) to the subject to treat the cancer; wherein the subject has: (i) an elevated level of adenosine A2A receptors when compared to a control; (ii) an elevated level of CD73 when compared to a control; and (iii) an elevated level of PD-L1 when compared to a control. In embodiments, the cancer is lung cancer, melanoma, breast cancer, colorectal cancer, bladder cancer, head and neck cancer, renal cell cancer, or prostate cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month. In embodiments, the methods of treating cancer in the subject further comprise administering a therapeutically effective amount of a chemotherapeutic agent.

The disclosure provides methods of treating lung cancer in a subject in need thereof by administering a therapeutically effective amount of a compound of Formula (III) to the subject to treat the lung cancer; wherein the subject has: (i) an elevated level of adenosine A2A receptors when compared to a control; (ii) an elevated level of CD73 when compared to a control; and (iii) an elevated level of PD-L1 when compared to a control. In embodiments, the lung cancer is non-small cell lung cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating melanoma in a subject in need thereof by administering a therapeutically effective amount of a compound of Formula (III) to the subject to treat the melanoma; wherein the subject has: (i) an elevated level of adenosine A2A receptors when compared to a control; (ii) an elevated level of CD73 when compared to a control; and (iii) an elevated level of PD-L1 when compared to a control. In embodiments, the melanoma is malignant melanoma. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating breast cancer in a subject in need thereof by administering a therapeutically effective amount of a compound of Formula (III) to the subject to treat the breast cancer; wherein the subject has: (i) an elevated level of adenosine A2A receptors when compared to a control; (ii) an elevated level of CD73 when compared to a control; and (iii) an elevated level of PD-L1 when compared to a control. In embodiments, the breast cancer is triple negative breast cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating colorectal cancer in a subject in need thereof by administering a therapeutically effective amount of a compound of Formula (III) to the subject to treat the colorectal cancer; wherein the subject has: (i) an elevated level of adenosine A2A receptors when compared to a control; (ii) an elevated level of CD73 when compared to a control; and (iii) an elevated level of PD-L1 when compared to a control. In embodiments, the colorectal cancer is microsatellite instable colorectal cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating bladder cancer in a subject in need thereof by administering a therapeutically effective amount of a compound of Formula (III) to the subject to treat the bladder cancer; wherein the subject has: (i) an elevated level of adenosine A2A receptors when compared to a control; (ii) an elevated level of CD73 when compared to a control; and (iii) an elevated level of PD-L1 when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating head and neck cancer in a subject in need thereof by administering a therapeutically effective amount of a compound of Formula (III) to the subject to treat the head and neck cancer; wherein the subject has: (i) an elevated level of adenosine A2A receptors when compared to a control; (ii) an elevated level of CD73 when compared to a control; and (iii) an elevated level of PD-L1 when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating renal cell cancer in a subject in need thereof by administering a therapeutically effective amount of a compound of Formula (III) to the subject to treat the renal cell cancer; wherein the subject has: (i) an elevated level of adenosine A2A receptors when compared to a control; (ii) an elevated level of CD73 when compared to a control; and (iii) an elevated level of PD-L1 when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating prostate cancer in a subject in need thereof by administering a therapeutically effective amount of a compound of Formula (III) to the subject to treat the prostate cancer; wherein the subject has: (i) an elevated level of adenosine A2A receptors when compared to a control; (ii) an elevated level of CD73 when compared to a control; and (iii) an elevated level of PD-L1 when compared to a control. In embodiments, the prostate cancer is castration-resistant prostate cancer or metastatic castration-resistant prostate cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating cancer in a subject in need thereof by administering a therapeutically effective amount of an adenosine pathway inhibitor to the subject to treat the cancer; wherein the subject has: (i) an elevated level of adenosine A2A receptors when compared to a control; and (ii) an elevated level of PD-L1 when compared to a control. In embodiments, the adenosine pathway inhibitor is an anti-CD73 compound. In embodiments, the adenosine pathway inhibitor is an anti-CD39 compound. In embodiments, the adenosine pathway inhibitor comprises an anti-CD73 compound and an anti-CD39 compound. In embodiments, the adenosine pathway inhibitor comprises an anti-CD73 compound and an adenosine A2A receptor antagonist. In embodiments, the adenosine pathway inhibitor comprises an adenosine A2A receptor antagonist and an anti-CD39 compound. In embodiments, the adenosine pathway inhibitor comprises an adenosine A2A receptor antagonist, an anti-CD73 compound, and an anti-CD39 compound. In embodiments, the adenosine pathway inhibitor is an adenosine A2A receptor antagonist. In embodiments, the adenosine A2A receptor antagonist is a compound of Formula (I) or a pharmaceutically acceptable salt thereof. In embodiments, the adenosine A2A receptor antagonist is a compound of Formula (II) or a pharmaceutically acceptable salt thereof. In embodiments, the adenosine A2A receptor antagonist is a compound of Formula (III) or a pharmaceutically acceptable salt thereof. In embodiments, the adenosine A2A receptor antagonist is a compound of Formula (IIIA) or a pharmaceutically acceptable salt thereof. In embodiments, the adenosine A2A receptor antagonist is a compound of Formula (IIIB) or a pharmaceutically acceptable salt thereof. In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the subject was responsive to prior PD-1 pathway inhibitor therapy. In embodiments, the subject is naïve to PD-1 pathway inhibitor therapy. In embodiments, the method of treating cancer is: (i) a method of increasing CD8-positive cells relative to the amount of regulatory T cells; (ii) a method of decreasing tumor volume; (iii) a method of enhancing anti-tumor immune memory; (iv) a method of treating a cancer tumor; or (v) two or more of the foregoing. In embodiments, the cancer is lung cancer. In embodiments, the lung cancer is non-small cell lung cancer. In embodiments, the cancer is melanoma. In embodiments, the melanoma is malignant melanoma. In embodiments, the cancer is breast cancer. In embodiments, the breast cancer is triple negative breast cancer. In embodiments, the cancer is colorectal cancer. In embodiments, the cancer is microsatellite instable colorectal cancer. In embodiments, the cancer is bladder cancer. In embodiments, the cancer is head and neck cancer. In embodiments, the cancer is renal cell cancer. In embodiments, the cancer is prostate cancer. In embodiments, the cancer is metastatic castration resistant prostate cancer. In embodiments, the methods of treating cancer in the subject further comprise administering a therapeutically effective amount of a chemotherapeutic agent.

The disclosure provides methods of treating cancer in a subject in need thereof by administering a therapeutically effective amount of a compound of Formula (III) to the subject to treat the cancer; wherein the subject has: (i) an elevated level of adenosine A2A receptors when compared to a control; and (ii) an elevated level of PD-L1 when compared to a control. In embodiments, the cancer is lung cancer, melanoma, breast cancer, colorectal cancer, bladder cancer, head and neck cancer, renal cell cancer, or prostate cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month. In embodiments, the methods of treating cancer in the subject further comprise administering a therapeutically effective amount of a chemotherapeutic agent.

The disclosure provides methods of treating lung cancer in a subject in need thereof by administering a therapeutically effective amount of a compound of Formula (III) to the subject to treat the lung cancer; wherein the subject has: (i) an elevated level of adenosine A2A receptors when compared to a control; and (ii) an elevated level of PD-L1 when compared to a control. In embodiments, the lung cancer is non-small cell lung cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating melanoma in a subject in need thereof by administering a therapeutically effective amount of a compound of Formula (III) to the subject to treat the melanoma; wherein the subject has: (i) an elevated level of adenosine A2A receptors when compared to a control; and (ii) an elevated level of PD-L1 when compared to a control. In embodiments, the melanoma is malignant melanoma. In embodiments, the compound of Formula (III) is in the form of a pharmaceutically acceptable salt. In embodiments, the compound of Formula (III) is a compound of Formula (IIIA). n embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating breast cancer in a subject in need thereof by administering a therapeutically effective amount of a compound of Formula (III) to the subject to treat the breast cancer; wherein the subject has: (i) an elevated level of adenosine A2A receptors when compared to a control; and (ii) an elevated level of PD-L1 when compared to a control. In embodiments, the breast cancer is triple negative breast cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating colorectal cancer in a subject in need thereof by administering a therapeutically effective amount of a compound of Formula (III) to the subject to treat the colorectal cancer; wherein the subject has: (i) an elevated level of adenosine A2A receptors when compared to a control; and (ii) an elevated level of PD-L1 when compared to a control. In embodiments, the colorectal cancer is microsatellite instable colorectal cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating bladder cancer in a subject in need thereof by administering a therapeutically effective amount of a compound of Formula (III) to the subject to treat the bladder cancer; wherein the subject has: (i) an elevated level of adenosine A2A receptors when compared to a control; and (ii) an elevated level of PD-L1 when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating head and neck cancer in a subject in need thereof by administering a therapeutically effective amount of a compound of Formula (III) to the subject to treat the head and neck cancer; wherein the subject has: (i) an elevated level of adenosine A2A receptors when compared to a control; and (ii) an elevated level of PD-L1 when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating renal cell cancer in a subject in need thereof by administering a therapeutically effective amount of a compound of Formula (III) to the subject to treat the renal cell cancer; wherein the subject has: (i) an elevated level of adenosine A2A receptors when compared to a control; and (ii) an elevated level of PD-L1 when compared to a control. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

The disclosure provides methods of treating prostate cancer in a subject in need thereof by administering a therapeutically effective amount of a compound of Formula (III) to the subject to treat the prostate cancer; wherein the subject has: (i) an elevated level of adenosine A2A receptors when compared to a control; and (ii) an elevated level of PD-L1 when compared to a control. In embodiments, the prostate cancer is castration-resistant prostate cancer or metastatic castration-resistant prostate cancer. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

Provided herein are methods of treating cancer in a subject by: (i) measuring an adenosine A2A receptor level in a biological sample obtained from the subject, and (ii) administering a therapeutically effective amount of an adenosine pathway inhibitor to the subject to treat the cancer. In embodiments, the methods of treating cancer in a subject comprise: (i) measuring an adenosine A2A receptor level and a CD73 level in a biological sample obtained from the subject, and (ii) administering a therapeutically effective amount of an adenosine pathway inhibitor to the subject to treat the cancer. In embodiments, the methods of treating cancer in a subject comprise: (i) measuring an adenosine A2A receptor level, a CD73 level, and a PD-L1 level in a biological sample obtained from the subject, and (ii) administering a therapeutically effective amount of an adenosine pathway inhibitor to the subject to treat the cancer. In embodiments, the methods of treating cancer in a subject comprise: (i) measuring an adenosine A2A receptor level and a PD-L1 level in a biological sample obtained from the subject, and (ii) administering a therapeutically effective amount of an adenosine pathway inhibitor to the subject to treat the cancer. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the subject was responsive to prior PD-1 pathway inhibitor therapy. In embodiments, the subject is naïve to PD-1 pathway inhibitor therapy. In embodiments, the adenosine pathway inhibitor is an anti-CD73 compound. In embodiments, the adenosine pathway inhibitor is an anti-CD39 compound. In embodiments, the adenosine pathway inhibitor comprises an anti-CD73 compound and an anti-CD39 compound. In embodiments, the adenosine pathway inhibitor comprises an anti-CD73 compound and an adenosine A2A receptor antagonist. In embodiments, the adenosine pathway inhibitor comprises an adenosine A2A receptor antagonist and an anti-CD39 compound. In embodiments, the adenosine pathway inhibitor comprises an adenosine A2A receptor antagonist, an anti-CD73 compound, and an anti-CD39 compound. In embodiments, the adenosine pathway inhibitor is an adenosine A2A receptor antagonist. In embodiments, the adenosine A2A receptor antagonist is a compound of Formula (I) or a pharmaceutically acceptable salt thereof. In embodiments, the adenosine A2A receptor antagonist is a compound of Formula (II) or a pharmaceutically acceptable salt thereof. In embodiments, the adenosine A2A receptor antagonist is a compound of Formula (III) or a pharmaceutically acceptable salt thereof. In embodiments, the adenosine A2A receptor antagonist is a compound of Formula (IIIA) or a pharmaceutically acceptable salt thereof. In embodiments, the adenosine A2A receptor antagonist is a compound of Formula (IIIB) or a pharmaceutically acceptable salt thereof. In embodiments, the method of treating cancer is: (i) a method of increasing CD8-positive cells III relative to the amount of regulatory T cells; (ii) a method of decreasing tumor volume; (iii) a method of enhancing anti-tumor immune memory; (iv) a method of treating a cancer tumor; or (v) two or more of the foregoing. In embodiments, the cancer is lung cancer. In embodiments, the lung cancer is non-small cell lung cancer. In embodiments, the cancer is melanoma. In embodiments, the melanoma is malignant melanoma. In embodiments, the cancer is breast cancer. In embodiments, the breast cancer is triple negative breast cancer. In embodiments, the cancer is colorectal cancer. In embodiments, the cancer is microsatellite instable colorectal cancer. In embodiments, the cancer is bladder cancer. In embodiments, the cancer is head and neck cancer. In embodiments, the cancer is renal cell cancer. In embodiments, the cancer is prostate cancer. In embodiments, the cancer is metastatic castration resistant prostate cancer. In embodiments, the methods of treating cancer in the subject further comprise administering a therapeutically effective amount of a chemotherapeutic agent.

In embodiments, the disclosure provides methods of treating cancer in a subject by: (i) measuring an adenosine A2A receptor level in a biological sample obtained from the subject, and (ii) administering a therapeutically effective amount of a compound of Formula (III) to the subject to treat the cancer. In embodiments, the methods of treating cancer in a subject comprise: (i) measuring an adenosine A2A receptor level and a CD73 level in a biological sample obtained from the subject, and (ii) administering a therapeutically effective amount of a compound of Formula (III) to the subject to treat the cancer. In embodiments, the methods of treating cancer in a subject comprise: (i) measuring an adenosine A2A receptor level, a CD73 level, and a PD-L1 level in a biological sample obtained from the subject, and (ii) administering a therapeutically effective amount of a compound of Formula (III) to the subject to treat the cancer. In embodiments, the methods of treating cancer in a subject comprise: (i) measuring an adenosine A2A receptor level and a PD-L1 level in a biological sample obtained from the subject, and (ii) administering a therapeutically effective amount of a compound of Formula (III) to the subject to treat the cancer. In embodiments, the cancer is lung cancer, melanoma, breast cancer, colorectal cancer, bladder cancer, head and neck cancer, renal cell cancer, or prostate cancer. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month. In embodiments, the methods of treating cancer in the subject further comprise administering a therapeutically effective amount of a chemotherapeutic agent.

In embodiments, the disclosure provides methods of treating lung cancer in a subject by: (i) measuring an adenosine A2A receptor level in a biological sample obtained from the subject, and (ii) administering a therapeutically effective amount of a compound of Formula (III) to the subject to treat the lung cancer. In embodiments, the methods of treating lung cancer in a subject comprise: (i) measuring an adenosine A2A receptor level and a CD73 level in a biological sample obtained from the subject, and (ii) administering a therapeutically effective amount of a compound of Formula (III) to the subject to treat the lung cancer. In embodiments, the methods of treating lung cancer in a subject comprise: (i) measuring an adenosine A2A receptor level, a CD73 level, and a PD-L1 level in a biological sample obtained from the subject, and (ii) administering a therapeutically effective amount of a compound of Formula (III) to the subject to treat the lung cancer. In embodiments, the methods of treating lung cancer in a subject comprise: (i) measuring an adenosine A2A receptor level and a PD-L1 level in a biological sample obtained from the subject, and (ii) administering a therapeutically effective amount of a compound of Formula (III) o the subject to treat the lung cancer. In embodiments, the lung cancer is non-small cell lung cancer. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

In embodiments, the disclosure provides methods of treating melanoma in a subject by: (i) measuring an adenosine A2A receptor level in a biological sample obtained from the subject, and (ii) administering a therapeutically effective amount of a compound of Formula (III) to the subject to treat the melanoma. In embodiments, the methods of treating melanoma in a subject comprise: (i) measuring an adenosine A2A receptor level and a CD73 level in a biological sample obtained from the subject, and (ii) administering a therapeutically effective amount of a compound of Formula (III) to the subject to treat the melanoma. In embodiments, the methods of treating melanoma in a subject comprise: (i) measuring an adenosine A2A receptor level, a CD73 level, and a PD-L1 level in a biological sample obtained from the subject, and (ii) administering a therapeutically effective amount of a compound of Formula (III) to the subject to treat the melanoma. In embodiments, the methods of treating melanoma in a subject comprise: (i) measuring an adenosine A2A receptor level and a PD-L1 level in a biological sample obtained from the subject, and (ii) administering a therapeutically effective amount of a compound of Formula (III) to the subject to treat the melanoma. In embodiments, the melanoma is malignant melanoma. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). n embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

In embodiments, the disclosure provides methods of treating breast cancer in a subject by: (i) measuring an adenosine A2A receptor level in a biological sample obtained from the subject, and (ii) administering a therapeutically effective amount of a compound of Formula (III) to the subject to treat the breast cancer. In embodiments, the methods of treating breast cancer in a subject comprise: (i) measuring an adenosine A2A receptor level and a CD73 level in a biological sample obtained from the subject, and (ii) administering a therapeutically effective amount of a compound of Formula (III) to the subject to treat the breast cancer. In embodiments, the methods of treating breast cancer in a subject comprise: (i) measuring an adenosine A2A receptor level, a CD73 level, and a PD-L1 level in a biological sample obtained from the subject, and (ii) administering a therapeutically effective amount of a compound of Formula (III) to the subject to treat the breast cancer. In embodiments, the methods of treating breast cancer in a subject comprise: (i) measuring an adenosine A2A receptor level and a PD-L1 level in a biological sample obtained from the subject, and (ii) administering a therapeutically effective amount of a compound of Formula (III) to the subject to treat the breast cancer. In embodiments, the breast cancer is triple negative breast cancer. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

In embodiments, the disclosure provides methods of treating colorectal cancer in a subject by: (i) measuring an adenosine A2A receptor level in a biological sample obtained from the subject, and (ii) administering a therapeutically effective amount of a compound of Formula (III) to the subject to treat the colorectal cancer. In embodiments, the methods of treating colorectal cancer in a subject comprise: (i) measuring an adenosine A2A receptor level and a CD73 level in a biological sample obtained from the subject, and (ii) administering a therapeutically effective amount of a compound of Formula (III) to the subject to treat the colorectal cancer. In embodiments, the methods of treating colorectal cancer in a subject comprise: (i) measuring an adenosine A2A receptor level, a CD73 level, and a PD-L1 level in a biological sample obtained from the subject, and (ii) administering a therapeutically effective amount of a compound of Formula (III) to the subject to treat the colorectal cancer. In embodiments, the methods of treating colorectal cancer in a subject comprise: (i) measuring an adenosine A2A receptor level and a PD-L1 level in a biological sample obtained from the subject, and (ii) administering a therapeutically effective amount of a compound of Formula (III) to the subject to treat the colorectal cancer. In embodiments, the colorectal cancer is microsatellite instable colorectal cancer. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

In embodiments, the disclosure provides methods of treating bladder cancer in a subject by: (i) measuring an adenosine A2A receptor level in a biological sample obtained from the subject, and (ii) administering a therapeutically effective amount of a compound of Formula (III) to the subject to treat the bladder cancer. In embodiments, the methods of treating bladder cancer in a subject comprise: (i) measuring an adenosine A2A receptor level and a CD73 level in a biological sample obtained from the subject, and (ii) administering a therapeutically effective amount of a compound of Formula (III) to the subject to treat the bladder cancer. In embodiments, the methods of treating bladder cancer in a subject comprise: (i) measuring an adenosine A2A receptor level, a CD73 level, and a PD-L1 level in a biological sample obtained from the subject, and (ii) administering a therapeutically effective amount of a compound of Formula (III) to the subject to treat the bladder cancer. In embodiments, the methods of treating bladder cancer in a subject comprise: (i) measuring an adenosine A2A receptor level and a PD-L1 level in a biological sample obtained from the subject, and (ii) administering a therapeutically effective amount of a compound of Formula (III) to the subject to treat the bladder cancer. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

In embodiments, the disclosure provides methods of treating head and neck cancer in a subject by: (i) measuring an adenosine A2A receptor level in a biological sample obtained from the subject, and (ii) administering a therapeutically effective amount of a compound of Formula (III) to the subject to treat the head and neck cancer. In embodiments, the methods of treating head and neck cancer in a subject comprise: (i) measuring an adenosine A2A receptor level and a CD73 level in a biological sample obtained from the subject, and (ii) administering a therapeutically effective amount of a compound of Formula (III) to the subject to treat the head and neck cancer. In embodiments, the methods of treating head and neck cancer in a subject comprise: (i) measuring an adenosine A2A receptor level, a CD73 level, and a PD-L1 level in a biological sample obtained from the subject, and (ii) administering a therapeutically effective amount of a compound of Formula (III) to the subject to treat the head and neck cancer. In embodiments, the methods of treating head and neck cancer in a subject comprise: (i) measuring an adenosine A2A receptor level and a PD-L1 level in a biological sample obtained from the subject, and (ii) administering a therapeutically effective amount of a compound of Formula (III) to the subject to treat the head and neck cancer. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

In embodiments, the disclosure provides methods of treating renal cell cancer in a subject by: (i) measuring an adenosine A2A receptor level in a biological sample obtained from the subject, and (ii) administering a therapeutically effective amount of a compound of Formula (III) to the subject to treat the renal cell cancer. In embodiments, the methods of treating renal cell cancer in a subject comprise: (i) measuring an adenosine A2A receptor level and a CD73 level in a biological sample obtained from the subject, and (ii) administering a therapeutically effective amount of a compound of Formula (III) to the subject to treat the renal cell cancer. In embodiments, the methods of treating renal cell cancer in a subject comprise: (i) measuring an adenosine A2A receptor level, a CD73 level, and a PD-L1 level in a biological sample obtained from the subject, and (ii) administering a therapeutically effective amount of a compound of Formula (III) to the subject to treat the renal cell cancer. In embodiments, the methods of treating renal cell cancer in a subject comprise: (i) measuring an adenosine A2A receptor level and a PD-L1 level in a biological sample obtained from the subject, and (ii) administering a therapeutically effective amount of a compound of Formula (III) to the subject to treat the renal cell cancer. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. n embodiments, the biological sample is a peripheral blood sample. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

In embodiments, the disclosure provides methods of treating prostate cancer in a subject by: (i) measuring an adenosine A2A receptor level in a biological sample obtained from the subject, and (ii) administering a therapeutically effective amount of a compound of Formula (III) to the subject to treat the prostate cancer. In embodiments, the methods of treating prostate cancer in a subject comprise: (i) measuring an adenosine A2A receptor level and a CD73 level in a biological sample obtained from the subject, and (ii) administering a therapeutically effective amount of a compound of Formula (III) to the subject to treat the prostate cancer. In embodiments, the methods of treating prostate cancer in a subject comprise: (i) measuring an adenosine A2A receptor level, a CD73 level, and a PD-L1 level in a biological sample obtained from the subject, and (ii) administering a therapeutically effective amount of a compound of Formula (III) to the subject to treat the prostate cancer. In embodiments, the methods of treating prostate cancer in a subject comprise: (i) measuring an adenosine A2A receptor level and a PD-L1 level in a biological sample obtained from the subject, and (ii) administering a therapeutically effective amount of a compound of Formula (III) to the subject to treat the prostate cancer. In embodiments, the prostate cancer is castration-resistant prostate cancer or metastatic castration-resistant prostate cancer. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the compound of Formula (III) is in the form of a pharmaceutically acceptable salt. In embodiments, the compound of Formula (III) is a compound of Formula (IIIA). In embodiments, the compound of Formula (III) is a compound of Formula (IIIB). In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the compound of Formula (III) is administered to the patient in an amount of about 75 mg to about 125 mg, once or twice daily. In embodiments, the compound of Formula (III) is administered to the patient in an amount of about 100 mg, twice daily. In embodiments, the treatment lasts for at least one month.

Provided here are methods to identify subjects who will be responsive to an adenosine pathway inhibitor, where the method comprises (i) obtaining a biological sample from the patient; and (ii) measuring an adenosine A2A receptor level in the biological sample; wherein if the adenosine A2A receptor level is elevated when compared to a control, the subject is identified as responsive to the adenosine pathway inhibitor. In embodiments, the methods to identify subjects who will be responsive to an adenosine pathway inhibitor comprise: (i) obtaining a biological sample from the patient; and (ii) measuring an adenosine A2A receptor level and a CD73 level in the biological sample; wherein if the adenosine A2A receptor level and the CD73 level are elevated when compared to a control, the subject is identified as responsive to the adenosine pathway inhibitor. In embodiments, the methods to identify subjects who will be responsive to an adenosine pathway inhibitor comprise: (i) obtaining a biological sample from the patient; and (ii) measuring an adenosine A2A receptor level, a CD73 level, and a PD-L1 level in the biological sample; wherein if the adenosine A2A receptor level, the CD73 level, and the PD-L1 level are elevated when compared to a control, the subject is identified as responsive to the adenosine pathway inhibitor. In embodiments, the methods to identify subjects who will be responsive to an adenosine pathway inhibitor comprise: (i) obtaining a biological sample from the patient; and (ii) measuring an adenosine A2A receptor level and a PD-L1 level in the biological sample; wherein if the adenosine A2A receptor level and the PD-L1 level are elevated when compared to a control, the subject is identified as responsive to the adenosine pathway inhibitor. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the subject was responsive to prior PD-1 pathway inhibitor therapy. In embodiments, the subject is naïve to PD-1 pathway inhibitor therapy. In embodiments, the adenosine pathway inhibitor is an anti-CD73 compound. In embodiments, the adenosine pathway inhibitor is an anti-CD39 compound. In embodiments, the adenosine pathway inhibitor comprises an anti-CD73 compound and an anti-CD39 compound. In embodiments, the adenosine pathway inhibitor comprises an anti-CD73 compound and an adenosine A2A receptor antagonist. In embodiments, the adenosine pathway inhibitor comprises an adenosine A2A receptor antagonist and an anti-CD39 compound. In embodiments, the adenosine pathway inhibitor comprises an adenosine A2A receptor antagonist, an anti-CD73 compound, and an anti-CD39 compound. In embodiments, the adenosine pathway inhibitor is an adenosine A2A receptor antagonist. In embodiments, the adenosine A2A receptor antagonist is a compound of Formula (I) or a pharmaceutically acceptable salt thereof. In embodiments, the adenosine A2A receptor antagonist is a compound of Formula (II) or a pharmaceutically acceptable salt thereof. In embodiments, the adenosine A2A receptor antagonist is a compound of Formula (III) or a pharmaceutically acceptable salt thereof. In embodiments, the adenosine A2A receptor antagonist is a compound of Formula (IIIA) or a pharmaceutically acceptable salt thereof. In embodiments, the adenosine A2A receptor antagonist is a compound of Formula (IIIB) or a pharmaceutically acceptable salt thereof. In embodiments, the subject has cancer. In embodiments, the cancer is lung cancer. In embodiments, the lung cancer is non-small cell lung cancer. In embodiments, the cancer is melanoma. In embodiments, the melanoma is malignant melanoma. In embodiments, the cancer is breast cancer. In embodiments, the breast cancer is triple negative breast cancer. In embodiments, the cancer is colorectal cancer. In embodiments, the cancer is microsatellite instable colorectal cancer. In embodiments, the cancer is bladder cancer. In embodiments, the cancer is head and neck cancer. In embodiments, the cancer is renal cell cancer. In embodiments, the cancer is prostate cancer. In embodiments, the cancer is metastatic castration resistant prostate cancer.

Provided here are methods to identify subjects who will be responsive to treatment with a compound of Formula (III), where the method comprises (i) obtaining a biological sample from the patient; and (ii) measuring an adenosine A2A receptor level in the biological sample; wherein if the adenosine A2A receptor level is elevated when compared to a control, the subject is identified as responsive to treatment with a compound of Formula (III). In embodiments, the methods to identify subjects who will be responsive to treatment with a compound of Formula (III) comprise: (i) obtaining a biological sample from the patient; and (ii) measuring an adenosine A2A receptor level and a CD73 level in the biological sample; wherein if the adenosine A2A receptor level and the CD73 level are elevated when compared to a control, the subject is identified as responsive to treatment with a compound of Formula (III). In embodiments, the methods to identify subjects who will be responsive treatment with a compound of Formula (III) comprise: (i) obtaining a biological sample from the patient; and (ii) measuring an adenosine A2A receptor level, a CD73 level, and a PD-L1 level in the biological sample; wherein if the adenosine A2A receptor level, the CD73 level, and the PD-L1 level are elevated when compared to a control, the subject is identified as responsive to treatment with a compound of Formula (III). In embodiments, the methods to identify subjects who will be responsive to treatment with a compound of Formula (III) comprise: (i) obtaining a biological sample from the patient; and (ii) measuring an adenosine A2A receptor level and a PD-L1 level in the biological sample; wherein if the adenosine A2A receptor level and the PD-L1 level are elevated when compared to a control, the subject is identified as responsive to treatment with a compound of Formula (III). In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the subject was responsive to prior PD-1 pathway inhibitor therapy. In embodiments, the subject is naïve to PD-1 pathway inhibitor therapy. In embodiments, the subject has cancer. In embodiments, the cancer is lung cancer. In embodiments, the lung cancer is non-small cell lung cancer. In embodiments, the cancer is melanoma. In embodiments, the melanoma is malignant melanoma. In embodiments, the cancer is breast cancer. In embodiments, the breast cancer is triple negative breast cancer. In embodiments, the cancer is colorectal cancer. In embodiments, the cancer is microsatellite instable colorectal cancer. In embodiments, the cancer is bladder cancer. In embodiments, the cancer is head and neck cancer. In embodiments, the cancer is renal cell cancer. In embodiments, the cancer is prostate cancer. In embodiments, the cancer is metastatic castration resistant prostate cancer.

Provided here are methods to select subjects for treatment with an adenosine pathway inhibitor, where the method comprises (i) obtaining a biological sample from the patient; and (ii) measuring an adenosine A2A receptor level in the biological sample; wherein if the adenosine A2A receptor level is elevated when compared to a control, the subject is selected for treatment with the adenosine pathway inhibitor. In embodiments, the method further comprises administering a therapeutically effective amount of the adenosine pathway inhibitor to treat cancer. In embodiments, the methods to select subjects for treatment with an adenosine pathway inhibitor comprise: (i) obtaining a biological sample from the patient; and (ii) measuring an adenosine A2A receptor level and a CD73 level in the biological sample; wherein if the adenosine A2A receptor level and the CD73 level are elevated when compared to a control, the subject is selected for treatment with the adenosine pathway inhibitor. In embodiments, the method further comprises administering a therapeutically effective amount of the adenosine pathway inhibitor to treat cancer. In embodiments, the methods to select subjects for treatment with an adenosine pathway inhibitor comprise: (i) obtaining a biological sample from the patient; and (ii) measuring an adenosine A2A receptor level, a CD73 level, and a PD-L1 level in the biological sample; wherein if the adenosine A2A receptor level, the CD73 level, and the PD-L1 level are elevated when compared to a control, the subject is selected for treatment with the adenosine pathway inhibitor. In embodiments, the method further comprises administering a therapeutically effective amount of the adenosine pathway inhibitor to treat cancer. In embodiments, the methods to select subjects for treatment with an adenosine pathway inhibitor comprise: (i) obtaining a biological sample from the patient; and (ii) measuring an adenosine A2A receptor level and a PD-L1 level in the biological sample; wherein if the adenosine A2A receptor level and the PD-L1 level are elevated when compared to a control, the subject is selected for treatment with the adenosine pathway inhibitor. In embodiments, the method further comprises administering a therapeutically effective amount of the adenosine pathway inhibitor to treat cancer. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the subject was responsive to prior PD-1 pathway inhibitor therapy. In embodiments, the subject is naïve to PD-1 pathway inhibitor therapy. In embodiments, the adenosine pathway inhibitor is an anti-CD73 compound. In embodiments, the adenosine pathway inhibitor is an anti-CD39 compound. In embodiments, the adenosine pathway inhibitor comprises an anti-CD73 compound and an anti-CD39 compound. In embodiments, the adenosine pathway inhibitor comprises an anti-CD73 compound and an adenosine A2A receptor antagonist. In embodiments, the adenosine pathway inhibitor comprises an adenosine A2A receptor antagonist and an anti-CD39 compound. In embodiments, the adenosine pathway inhibitor comprises an adenosine A2A receptor antagonist, an anti-CD73 compound, and an anti-CD39 compound. In embodiments, the adenosine pathway inhibitor is an adenosine A2A receptor antagonist. In embodiments, the adenosine A2A receptor antagonist is a compound of Formula (I) or a pharmaceutically acceptable salt thereof. In embodiments, the adenosine A2A receptor antagonist is a compound of Formula (II) or a pharmaceutically acceptable salt thereof. In embodiments, the adenosine A2A receptor antagonist is a compound of Formula (III) or a pharmaceutically acceptable salt thereof. In embodiments, the adenosine A2A receptor antagonist is a compound of Formula (IIIA) or a pharmaceutically acceptable salt thereof. In embodiments, the adenosine A2A receptor antagonist is a compound of Formula (IIIB) or a pharmaceutically acceptable salt thereof. In embodiments, the subject has cancer. In embodiments, the cancer is lung cancer. In embodiments, the lung cancer is non-small cell lung cancer. In embodiments, the cancer is melanoma. In embodiments, the melanoma is malignant melanoma. In embodiments, the cancer is breast cancer. In embodiments, the breast cancer is triple negative breast cancer. In embodiments, the cancer is colorectal cancer. In embodiments, the cancer is microsatellite instable colorectal cancer. In embodiments, the cancer is bladder cancer. In embodiments, the cancer is head and neck cancer. In embodiments, the cancer is renal cell cancer. In embodiments, the cancer is prostate cancer. In embodiments, the cancer is metastatic castration resistant prostate cancer.

Provided here are methods to select subjects for treatment with a compound of Formula (III), where the method comprises (i) obtaining a biological sample from the patient; and (ii) measuring an adenosine A2A receptor level in the biological sample; wherein if the adenosine A2A receptor level is elevated when compared to a control, the subject is selected for treatment with a compound of Formula (III). In embodiments, the method further comprises administering a therapeutically effective amount of a compound of Formula (III) to treat cancer. In embodiments, the methods to select subjects for treatment with a compound of Formula (III) comprise: (i) obtaining a biological sample from the patient; and (ii) measuring an adenosine A2A receptor level and a CD73 level in the biological sample; wherein if the adenosine A2A receptor level and the CD73 level are elevated when compared to a control, the subject is selected for treatment with a compound of Formula (III). In embodiments, the method further comprises administering a therapeutically effective amount of a compound of Formula (III) to treat cancer. In embodiments, the methods to select subjects for treatment with a compound of Formula (III) comprise: (i) obtaining a biological sample from the patient; and (ii) measuring an adenosine A2A receptor level, a CD73 level, and a PD-L1 level in the biological sample; wherein if the adenosine A2A receptor level, the CD73 level, and the PD-L1 level are elevated when compared to a control, the subject is selected for treatment with a compound of Formula (III). In embodiments, the method further comprises administering a therapeutically effective amount of a compound of Formula (III) to treat cancer. In embodiments, the methods to select subjects for treatment with a compound of Formula (III) comprise: (i) obtaining a biological sample from the patient; and (ii) measuring an adenosine A2A receptor level and a PD-L1 level in the biological sample; wherein if the adenosine A2A receptor level and the PD-L1 level are elevated when compared to a control, the subject is selected for treatment with a compound of Formula (III). In embodiments, the method further comprises administering a therapeutically effective amount of a compound of Formula (III) to treat cancer. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the subject was responsive to prior PD-1 pathway inhibitor therapy. In embodiments, the subject is naïve to PD-1 pathway inhibitor therapy. In embodiments, the subject has cancer. In embodiments, the cancer is lung cancer. In embodiments, the lung cancer is non-small cell lung cancer. In embodiments, the cancer is melanoma. In embodiments, the melanoma is malignant melanoma. In embodiments, the cancer is breast cancer. In embodiments, the breast cancer is triple negative breast cancer. In embodiments, the cancer is colorectal cancer. In embodiments, the cancer is microsatellite instable colorectal cancer. In embodiments, the cancer is bladder cancer. In embodiments, the cancer is head and neck cancer. In embodiments, the cancer is renal cell cancer. In embodiments, the cancer is prostate cancer. In embodiments, the cancer is metastatic castration resistant prostate cancer.

Provided herein are methods to determine whether a cancer patient expresses high adenosine A2A receptor levels by: (i) obtaining a biological sample from the patient; and (ii) measuring the adenosine A2A receptor levels in the biological sample. The method may further comprise administering a therapeutically effective amount of an adenosine pathway inhibitor. Provided herein are methods to determine whether a cancer patient expresses high adenosine A2A receptor levels and high CD73 levels by: (i) obtaining a biological sample from the patient; and (ii) measuring the adenosine A2A receptor levels and the CD73 levels in the biological sample. The method may further comprise administering a therapeutically effective amount of an adenosine pathway inhibitor. Provided herein are methods to determine whether a cancer patient expresses high adenosine A2A receptor levels, high CD73 levels, and high PD-L1 levels by: (i) obtaining a biological sample from the patient; and (ii) measuring the adenosine A2A receptor levels, the CD73 levels, and the PD-L1 levels in the biological sample. The method may further comprise administering a therapeutically effective amount of an adenosine pathway inhibitor. Provided herein are methods to determine whether a cancer patient expresses high adenosine A2A receptor levels and high PD-L1 levels by: (i) obtaining a biological sample from the patient; and (ii) measuring the adenosine A2A receptor levels and the PD-L1 levels in the biological sample. The method may further comprise administering a therapeutically effective amount of an adenosine pathway inhibitor. In embodiments, the biological sample is a tumor sample. In embodiments, the biological sample is a resected tumor sample. In embodiments, the biological sample is a resected tumor sample from a primary tumor. In embodiments, the biological sample is a resected tumor sample from a metastisic tumor. In embodiments, the biological sample is a tumor biopsy sample. In embodiments, the biological sample is a tumor biopsy sample from a primary tumor. In embodiments, the biological sample is a tumor biopsy sample from a metastisic tumor. In embodiments, the biological sample is a blood sample. In embodiments, the biological sample is a peripheral blood sample. In embodiments, the subject has been previously treated with PD-1 pathway inhibitor therapy, such as a PD-1 inhibitor and/or a PD-L1 inhibitor. In embodiments, the subject is an anti-PD-1 refractory subject. In embodiments, the subject is an anti-PD-1 resistant subject. In embodiments, the subject was responsive to prior PD-1 pathway inhibitor therapy. In embodiments, the subject is naïve to PD-1 pathway inhibitor therapy. In embodiments, the adenosine pathway inhibitor is an anti-CD73 compound. In embodiments, the adenosine pathway inhibitor is an anti-CD39 compound. In embodiments, the adenosine pathway inhibitor comprises an anti-CD73 compound and an anti-CD39 compound. In embodiments, the adenosine pathway inhibitor comprises an anti-CD73 compound and an adenosine A2A receptor antagonist. In embodiments, the adenosine pathway inhibitor comprises an adenosine A2A receptor antagonist and an anti-CD39 compound. In embodiments, the adenosine pathway inhibitor comprises an adenosine A2A receptor antagonist, an anti-CD73 compound, and an anti-CD39 compound. In embodiments, the adenosine pathway inhibitor is an adenosine A2A receptor antagonist. In embodiments, the adenosine A2A receptor antagonist is a compound of Formula (I) or a pharmaceutically acceptable salt thereof. In embodiments, the adenosine A2A receptor antagonist is a compound of Formula (II) or a pharmaceutically acceptable salt thereof. In embodiments, the adenosine A2A receptor antagonist is a compound of Formula (III) or a pharmaceutically acceptable salt thereof. In embodiments, the adenosine A2A receptor antagonist is a compound of Formula (IIIA) or a pharmaceutically acceptable salt thereof. In embodiments, the adenosine A2A receptor antagonist is a compound of Formula (IIIB) or a pharmaceutically acceptable salt thereof. In embodiments, the subject has cancer. In embodiments, the cancer is lung cancer. In embodiments, the lung cancer is non-small cell lung cancer. In embodiments, the cancer is melanoma. In embodiments, the melanoma is malignant melanoma. In embodiments, the cancer is breast cancer. In embodiments, the breast cancer is triple negative breast cancer. In embodiments, the cancer is colorectal cancer. In embodiments, the cancer is microsatellite instable colorectal cancer. In embodiments, the cancer is bladder cancer. In embodiments, the cancer is head and neck cancer. In embodiments, the cancer is renal cell cancer. In embodiments, the cancer is prostate cancer. In embodiments, the cancer is metastatic castration resistant prostate cancer.

Dose and Dosing Regimens

The dosage and frequency (single or multiple doses) of the adenosine pathway inhibitor administered to a subject can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer and severity of such symptoms), kind of concurrent treatment, complications from the disease being treated or other health-related problems. Other therapeutic regimens or agents can be used in conjunction with the methods and adenosine pathway inhibitors described herein. Adjustment and manipulation of established dosages (e.g., frequency and duration) are well within the ability of those skilled in the art.

For any composition and adenosine pathway inhibitor described herein, the therapeutically effective amount can be initially determined from cell culture assays. Target concentrations will be those concentrations of adenosine pathway inhibitor s that are capable of achieving the methods described herein, as measured using the methods described herein or known in the art. As is well known in the art, effective amounts of adenosine pathway inhibitors for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the adenosine pathway inhibitors may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Generally, treatment is initiated with smaller dosages which are less than the optimum dose of the adenosine pathway inhibitor. Thereafter, the dosage is increased by small increments until the optimum effect under circumstances is reached. Dosage amounts and intervals can be adjusted individually to provide levels of the adenosine pathway inhibitor effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of adenosine pathway inhibitor by considering factors such as compound potency, relative bioavailability, patient body weight, presence and severity of adverse side effects.

In embodiments, the adenosine pathway inhibitor is an adenosine A2A receptor antagonist. In embodiments, the adenosine pathway inhibitor is a compound of Formula (I) or a pharmaceutically acceptable salt thereof. In embodiments, the adenosine pathway inhibitor is a compound of Formula (II) or a pharmaceutically acceptable salt thereof. In embodiments, the adenosine pathway inhibitor is a compound of Formula (III) or a pharmaceutically acceptable salt thereof. In embodiments, the adenosine pathway inhibitor is a compound of Formula (IIIA) or a pharmaceutically acceptable salt thereof. In embodiments, the adenosine pathway inhibitor is a compound of Formula (IIIB) or a pharmaceutically acceptable salt thereof. The compounds of Formula (I), Formula (II), Formula (III), Formula (IIIA), and Formula (IIIB) may also be referred to as A2A receptor antagonists or adenosine A2A receptor antagonists.

In embodiments, the A2A receptor antagonist is administered at an amount of about 0.5 mg/kg, 1 mg/kg, 2 mg/kg, 3 mg/kg, 4 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg or 300 mg/kg. In embodiments, the A2A receptor antagonist is administered at an amount of about 0.5 mg/kg. In embodiments, the A2A receptor antagonist is administered at an amount of about 1 mg/kg. In embodiments, the A2A receptor antagonist is administered at an amount of about 5 mg/kg. In embodiments, the A2A receptor antagonist is administered at an amount of about 10 mg/kg. In embodiments, the A2A receptor antagonist is administered at an amount of about 20 mg/kg. In embodiments, the A2A receptor antagonist is administered at an amount of about 30 mg/kg. In embodiments, the A2A receptor antagonist is administered at an amount of about 40 mg/kg. In embodiments, the A2A receptor antagonist is administered at an amount of about 50 mg/kg. In embodiments, the A2A receptor antagonist is administered at an amount of about 60 mg/kg. In embodiments, the A2A receptor antagonist is administered at an amount of about 70 mg/kg. In embodiments, the A2A receptor antagonist is administered at an amount of about 80 mg/kg. In embodiments, the A2A receptor antagonist is administered at an amount of about 90 mg/kg. In embodiments, the A2A receptor antagonist is administered at an amount of about 100 mg/kg. In embodiments, the A2A receptor antagonist is administered at an amount of about 200 mg/kg. In embodiments, the A2A receptor antagonist is administered at an amount of about 300 mg/kg. It is understood that where the amount is referred to as “mg/kg”, the amount is milligram per kilogram body weight of the subject being administered with the A2A receptor antagonist.

In embodiments, the A2A receptor antagonist is administered at an amount of about 0.5 mg/kg, 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg or 300 mg/kg. In embodiments, the A2A receptor antagonist is administered at an amount of about 1 mg/kg. In embodiments, the A2A receptor antagonist is administered at an amount of about 1 mg/kg to 2 mg/kg. In embodiments, the A2A receptor antagonist is administered at an amount of about 1 mg/kg to 3 mg/kg. In embodiments, the A2A receptor antagonist is administered at an amount of about 1 mg/kg to 4 mg/kg. In embodiments, the A2A receptor antagonist is administered at an amount of about 1 mg/kg to 5 mg/kg.

In embodiments, the A2A receptor antagonist is administered at an amount of about 10 mg BID, 20 mg BID, 30 mg BID, 40 mg BID, 50 mg BID, 60 mg BID, 70 mg BID, 80 mg BID, 90 mg BID, 100 mg BID, 110 mg BID, 120 mg BID, 130 mg BID, 140 mg BID, 150 mg BID, 160 mg BID, 170 mg BID, 180 mg BID, 190 mg BID, 200 mg BID, 210 mg BID, 220 mg BID, 230 mg BID, 240 mg BID, 250 mg BID, 260 mg BID, 270 mg BID, 280 mg BID, 290 mg BID, or 300 mg BID. In embodiments, the A2A receptor antagonist is administered at an amount of about 10 mg BID. In embodiments, the A2A receptor antagonist is administered at an amount of about 20 mg BID. In embodiments, the A2A receptor antagonist is administered at an amount of about 30 mg BID. In embodiments, the A2A receptor antagonist is administered at an amount of about 40 mg BID. In embodiments, the A2A receptor antagonist is administered at an amount of about 50 mg BID. In embodiments, the A2A receptor antagonist is administered at an amount of about 60 mg BID. In embodiments, the A2A receptor antagonist is administered at an amount of about 70 mg BID. In embodiments, the A2A receptor antagonist is administered at an amount of about 80 mg BID. In embodiments, the A2A receptor antagonist is administered at an amount of about 90 mg BID. In embodiments, the A2A receptor antagonist is administered at an amount of about 100 mg BID. In embodiments, the A2A receptor antagonist is administered at an amount of about 110 mg BID. In embodiments, the A2A receptor antagonist is administered at an amount of about 120 mg BID. In embodiments, the A2A receptor antagonist is administered at an amount of about 130 mg BID. In embodiments, the A2A receptor antagonist is administered at an amount of about 140 mg BID. In embodiments, the A2A receptor antagonist is administered at an amount of about 150 mg BID. In embodiments, the A2A receptor antagonist is administered at an amount of about 160 mg BID. In embodiments, the A2A receptor antagonist is administered at an amount of about 170 mg BID. In embodiments, the A2A receptor antagonist is administered at an amount of about 180 mg BID. In embodiments, the A2A receptor antagonist is administered at an amount of about 190 mg BID. In embodiments, the A2A receptor antagonist is administered at an amount of about 200 mg BID. In embodiments, the A2A receptor antagonist is administered at an amount of about 210 mg BID. In embodiments, the A2A receptor antagonist is administered at an amount of about 220 mg BID. In embodiments, the A2A receptor antagonist is administered at an amount of about 230 mg BID. In embodiments, the A2A receptor antagonist is administered at an amount of about 240 mg BID. In embodiments, the A2A receptor antagonist is administered at an amount of about 250 mg BID. In embodiments, the A2A receptor antagonist is administered at an amount of about 260 mg BID. In embodiments, the A2A receptor antagonist is administered at an amount of about 270 mg BID. In embodiments, the A2A receptor antagonist is administered at an amount of about 280 mg BID. In embodiments, the A2A receptor antagonist is administered at an amount of about 290 mg BID. In embodiments, the A2A receptor antagonist is administered at an amount of about 300 mg BID. It is understood that where the amount is referred to as “BID” which stands for “bis in die”, the amount is administered twice a day.

In embodiments, the A2A receptor antagonist is administered at an amount of about 10 mg QD, 20 mg QD, 30 mg QD, 40 mg QD, 50 mg QD, 60 mg QD, 70 mg QD, 80 mg QD, 90 mg QD, 100 mg QD, 110 mg QD, 120 mg QD, 130 mg QD, 140 mg QD, 150 mg QD, 160 mg QD, 170 mg QD, 180 mg QD, 190 mg QD, 200 mg QD, 210 mg QD, 220 mg QD, 230 mg QD, 240 mg QD, 250 mg QD, 260 mg QD, 270 mg QD, 280 mg QD, 290 mg QD, or 300 mg QD. In embodiments, the A2A receptor antagonist is administered at an amount of about 10 mg QD. In embodiments, the A2A receptor antagonist is administered at an amount of about 20 mg QD. In embodiments, the A2A receptor antagonist is administered at an amount of about 30 mg QD. In embodiments, the A2A receptor antagonist is administered at an amount of about 40 mg QD. In embodiments, the A2A receptor antagonist is administered at an amount of about 50 mg QD. In embodiments, the A2A receptor antagonist is administered at an amount of about 60 mg QD. In embodiments, the A2A receptor antagonist is administered at an amount of about 70 mg QD. In embodiments, the A2A receptor antagonist is administered at an amount of about 80 mg QD. In embodiments, the A2A receptor antagonist is administered at an amount of about 90 mg QD. In embodiments, the A2A receptor antagonist is administered at an amount of about 100 mg QD. In embodiments, the A2A receptor antagonist is administered at an amount of about 110 mg QD. In embodiments, the A2A receptor antagonist is administered at an amount of about 120 mg QD. In embodiments, the A2A receptor antagonist is administered at an amount of about 130 mg QD. In embodiments, the A2A receptor antagonist is administered at an amount of about 140 mg QD. In embodiments, the A2A receptor antagonist is administered at an amount of about 150 mg QD. In embodiments, the A2A receptor antagonist is administered at an amount of about 160 mg QD. In embodiments, the A2A receptor antagonist is administered at an amount of about 170 mg QD. In embodiments, the A2A receptor antagonist is administered at an amount of about 180 mg QD. In embodiments, the A2A receptor antagonist is administered at an amount of about 190 mg QD. In embodiments, the A2A receptor antagonist is administered at an amount of about 200 mg QD. In embodiments, the A2A receptor antagonist is administered at an amount of about 210 mg QD. In embodiments, the A2A receptor antagonist is administered at an amount of about 220 mg QD. In embodiments, the A2A receptor antagonist is administered at an amount of about 230 mg QD. In embodiments, the A2A receptor antagonist is administered at an amount of about 240 mg QD. In embodiments, the A2A receptor antagonist is administered at an amount of about 250 mg QD. In embodiments, the A2A receptor antagonist is administered at an amount of about 260 mg QD. In embodiments, the A2A receptor antagonist is administered at an amount of about 270 mg QD. In embodiments, the A2A receptor antagonist is administered at an amount of about 280 mg QD. In embodiments, the A2A receptor antagonist is administered at an amount of about 290 mg QD. In embodiments, the A2A receptor antagonist is administered at an amount of about 300 mg QD. It is understood that where the amount is referred to as “QD” which stands for “quaque die”, the amount is administered once a day.

The A2A receptor antagonist may be administered at an amount as provided herein on 28 consecutive days. The A2A receptor antagonist may be administered at an amount as provided herein on 14 consecutive days. In embodiments, the A2A receptor antagonist is administered at 50 mg BID, 100 mg BID or 200 mg QD. In embodiments, the A2A receptor antagonist is administered at 50 mg BID. In embodiments, the A2A receptor antagonist is administered at 100 mg BID. In embodiments, the A2A receptor antagonist is administered at 200 mg QD.

In embodiments, the A2A receptor antagonist is administered at an amount of about 0.5 mg/kg, 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg or 300 mg/kg. In embodiments, the A2A receptor antagonist is administered at an amount of about 1 mg/kg.

Pharmaceutical Compositions

Provided herein are pharmaceutical compositions comprising an adenosine pathway inhibitor and a pharmaceutically acceptable excipient. The term “active ingredient” refers to adenosine pathway inhibitors. The provided compositions are suitable for formulation and administration in vitro or in vivo. Suitable carriers and excipients and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

Compositions can be administered for therapeutic or prophylactic treatments. In therapeutic applications, compositions are administered to a patient suffering from a disease (e.g., cancer) in a “therapeutically effective dose.” Amounts effective for this use will depend upon the severity of the disease and the general state of the patient's health. Single or multiple administrations of the compositions may be administered depending on the dosage and frequency as required and tolerated by the patient.

Pharmaceutical compositions provided herein include compositions wherein the active ingredient (e.g. compositions described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, the compounds described herein will contain an amount of active ingredient effective to achieve the desired result, e.g., modulating the activity of a target molecule, and/or reducing, eliminating, or slowing the progression of disease symptoms. Determination of a therapeutically effective amount of a compound described herein is well within the capabilities of those skilled in the art, especially in light of the detailed disclosure herein.

Provided compositions can include a single agent or more than one agent. The compositions for administration will commonly include an agent as described herein dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary, and will be selected primarily based on fluid volumes, viscosities, body weight and the like in accordance with the particular mode of administration selected and the subject's needs.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In some embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active compounds in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. DMSO can be used as solvent for extremely rapid penetration, delivering high concentrations of the active agents to a small area.

The formulations of compounds can be presented in unit-dose or multi-dose sealed containers, such as ampules and vials. Thus, the composition can be in unit dosage form. In such form the preparation is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

“Pharmaceutically acceptable excipient” and “pharmaceutically acceptable carrier” refer to a substance that aids the administration of an active agent to and absorption by a subject and can be included in the compositions herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

In one aspect, a pharmaceutical composition including an A2A receptor antagonist and a pharmaceutically acceptable excipient is provided.

Additional Therapeutic Agents

In embodiments of the methods described herein, the patient is administered a therapeutically effective amount of an adenosine pathway inhibitor described herein, but is not administered or treated with any other active agents. In embodiments of the methods of described herein, the patient is administered therapeutically effective amount of: (i) an adenosine pathway inhibitor described herein, and (ii) a chemotherapeutic agent. In embodiments of the methods of described herein, the patient is administered therapeutically effective amount of: (i) an adenosine A2A receptor antagonist, and (ii) an anti-CD73 compound. In embodiments of the methods of described herein, the patient is administered therapeutically effective amount of: (i) an adenosine A2A receptor antagonist, (ii) an anti-CD73 compound, and (ii) a chemotherapeutic agent. In embodiments of the methods described herein, the patient is not administered or treated with an anti-CD73 compound, such as an anti-CD73 antibody. In embodiments of the methods described herein, the patient is not administered or treated with an anti-CD39 compound, such as an anti-CD39 antibody. In embodiments of the methods described herein, the patient is not administered or treated with an anti-CD73 compound or an anti-CD39 compound. In embodiments of the methods described herein, the patient is not administered or treated with an anti-CD73 compound, an anti-CD39 compound, or a PD-1 pathway inhibitor. In embodiments of the methods described herein, the patient is not administered or treated with a PD-1 pathway inhibitor, such a PD-1 inhibitor or a PD-L1 inhibitor.

The additional therapeutic agent useful for the methods provided herein may be a compound, drug, antagonist, inhibitor, or modulator, having antineoplastic properties or the ability to inhibit the growth or proliferation of cells. In embodiments, the additional therapeutic agent is a chemotherapeutic. “Chemotherapeutic” or “chemotherapeutic agent” is used in accordance with its plain ordinary meaning and refers to a chemical composition or compound having antineoplastic properties or the ability to inhibit the growth or proliferation of cells. In embodiments, the second therapeutic agent is radiation therapy. In embodiments, the third therapeutic agent is an agent approved by the FDA or similar regulatory agency of a country other than the USA, for treating cancer.

In the provided methods of treatment, additional therapeutic agents can be used that are suitable to the disease (e.g., cancer) being treated. Suitable additional therapeutic agents include, but are not limited to analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopaminergics, hematological agents, immunological agents, muscarinics, protease inhibitors, vitamins, growth factors, and hormones. The choice of agent and dosage can be determined readily by one of skill in the art based on the given disease being treated.

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

The combined administrations contemplates co-administration, using separate formulations or a single pharmaceutical formulation, and consecutive administration in either order, wherein preferably there is a time period while both (or all) active agents simultaneously exert their biological activities.

Detection, Assay, and Diagnostic Methods

In embodiments, methods described herein may include detecting a level of, e.g., adenosine A2A receptors, CD73, PD-L1, e.g., with a specific binding agent (e.g., an agent that binds to a protein or nucleic acid molecule). Exemplary binding agents include an antibody or a fragment thereof, a detectable protein or a fragment thereof, a nucleic acid molecule such as an oligonucleotide/polynucleotide comprising a sequence that is complementary to patient genomic DNA, mRNA or a cDNA produced from patient mRNA, or any combination thereof. In embodiments, an antibody is labeled with detectable moiety, e.g., a fluorescent compound, an enzyme or functional fragment thereof, or a radioactive agent. In embodiments, an antibody is detectably labeled by coupling it to a chemiluminescent compound. In embodiments, the presence of the chemiluminescent-tagged antibody is then determined by detecting the presence of luminescence that arises during the course of chemical reaction. Non-limiting examples of particularly useful chemiluminescent labeling compounds are luminol, isoluminol, theromatic acridinium ester, imidazole, acridinium salt and oxalate ester.

In embodiments, a specific binding agent is an agent that has greater than 10-fold, preferably greater than 100-fold, and most preferably, greater than 1000-fold affinity for the target molecule as compared to another molecule. As the skilled artisan will appreciate the term specific is used to indicate that other biomolecules present in the sample do not significantly bind to the binding agent specific for the target molecule. In embodiments, the level of binding to a biomolecule other than the target molecule results in a binding affinity which is at most only 10% or less, only 5% or less only 2% or less or only 1% or less of the affinity to the target molecule, respectively. A preferred specific binding agent will fulfill both the above minimum criteria for affinity as well as for specificity. For example, in embodiments an antibody has a binding affinity (e.g., Kd) in the low micromolar (10⁻⁶), nanomolar (10⁻⁷-10⁻⁹), with high affinity antibodies in the low nanomolar (10⁻⁹) or pico molar (10⁻¹²) range for its specific target ligand.

In embodiments, the present subject matter provides a composition comprising a binding agent, wherein the binding agent is attached to a solid support, (e.g., a strip, a polymer, a bead, a nanoparticle, a plate such as a multiwell plate, or an array such as a microarray). In embodiments relating to the use of a nucleic acid probe attached to a solid support (such as a microarray), a nucleic acid in a test sample may be amplified (e.g., using PCR) before or after the nucleic acid to be measured is hybridized with the probe. In embodiments, reverse transcription polymerase chain reaction (RT-PCR) is used to detect mRNA levels. In embodiments, a probe on a solid support is used, and mRNA (or a portion thereof) in a biological sample is converted to cDNA or partial cDNA and then the cDNA or partial cDNA is hybridized to a probe (e.g., on a microarray), hybridized to a probe and then amplified, or amplified and then hybridized to a probe. In embodiments, a strip may be a nucleic acid-probe coated porous or non-porous solid support strip comprising linking a nucleic acid probe to a carrier to prepare a conjugate and immobilizing the conjugate on a porous solid support. In embodiments, the support or carrier comprises glass, polystyrene, polypropylene, polyethylene, dextran, nylon, amylases, natural and modified celluloses, polyacrylamides, gabbros, and magnetite. In embodiments, the nature of the carrier can be either soluble to some extent or insoluble for the purposes of the present subject matter. In embodiments, the support material may have any structural configuration so long as the coupled molecule is capable of binding to a binding agent (e.g., an antibody). In embodiments, the support configuration may be spherical, as in a bead, or cylindrical, as in the inside surface of a test tube, or the external surface of a rod. In embodiments, the surface may be flat such as a plate (or a well within a multiwell plate), sheet, or test strip, etc. polystyrene beads. Those skilled in the art will know many other suitable carriers for binding antibody or antigen, or will be able to ascertain the same by use of routine experimentation.

In embodiments, a solid support comprises a polymer, to which an agent is chemically bound, immobilized, dispersed, or associated. In embodiments, a polymer support may be, e.g., a network of polymers, and may be prepared in bead form (e.g., by suspension polymerization). In embodiments, the location of active sites introduced into a polymer support depends on the type of polymer support. In embodiments, in a swollen-gel-bead polymer support the active sites are distributed uniformly throughout the beads, whereas in a macroporous-bead polymer support they are predominantly on the internal surfaces of the macropores. In embodiments, the solid support, e.g., a device, may contain an adenosine A2A receptor binding agent alone or together with a binding agent for at least one, two, three or more other molecules, e.g., CD73, PD-L1 or both.

In embodiments, detection is accomplished using an ELISA or Western blot format. In embodiments, the binding agent comprises an nucleic acid (e.g., a probe or primers that are complementary for mRNA or cDNA), and the detecting step is accomplished using a polymerase chain reaction (PCR) or Northern blot format, or other means of detection. In embodiments, a probe or primer is about 10-20, 15-25, 15-35, 15-25, 20-80, 50-100, or 10-100 nucleotides in length, e.g., about 10, 12, 15, 20, 25, 30, 35, 40, 45, 50, 55, 60, 70, 80, 90, or 100 nucleotides in length or less than about 30, 35, 40, 45, 50, 55, 60, 70, 80, 90, or 100 nucleotides in length.

As used herein, “assaying” means using an analytic procedure to qualitatively assess or quantitatively measure the presence or amount or the functional activity of a target entity. For example, assaying the level of a compound (such as a protein or an mRNA molecule) means using an analytic procedure (such as an in vitro procedure) to qualitatively assess or quantitatively measure the presence or amount of the compound.

In embodiments, the cells in a biological sample are lysed to release a protein or nucleic acid. Numerous methods for lysing cells and assessing protein and nucleic acid levels are known in the art. In embodiments, cells are physically lysed, such as by mechanical disruption, liquid homogenization, high frequency sound waves, freeze/thaw cycles, with a detergent, or manual grinding. Non-limiting examples of detergents include Tween 20, Triton X-100, and Sodium Dodecyl Sulfate (SDS). Non-limiting examples of assays for determining the level of a protein include HPLC, LC/MS, ELISA, immunoelectrophoresis, Western blot, immunohistochemistry, and radioimmuno assays. Non-limiting examples of assays for determining the level of an mRNA include Northern blotting, RT-PCR, RNA sequencing, and qRT-PCR.

In embodiments, the tumor sample can be obtained by a variety of procedures including, but not limited to, surgical excision, aspiration or biopsy. In embodiments, the tissue sample may be sectioned and assayed as a fresh specimen; alternatively, the tissue sample may be frozen for further sectioning. In embodiments, the tissue sample is preserved by fixing and embedding in paraffin or the like.

In embodiments, once a suitable biological sample (e.g., tumor) has been obtained, it is analyzed to quantitate the expression level of each of the genes, e.g. adenosine A2A receptors, CD73, PD-L1, and the like. In embodiments, determining the expression level of a gene comprises detecting and quantifying RNA transcribed from that gene or a protein translated from such RNA. In embodiments, the RNA includes mRNA transcribed from the gene, and/or specific spliced variants thereof and/or fragments of such mRNA and spliced variants.

In embodiments, raw expression values are normalized by performing quantile normalization relative to the reference distribution and subsequent log 10-transformation. In embodiments, when the gene expression is detected using the nCounter® Analysis System marketed by NanoString® Technologies, the reference distribution is generated by pooling reported (i.e., raw) counts for the test sample and one or more control samples (preferably at least 2 samples, more preferably at least any of 4, 8 or 16 samples) after excluding values for technical (both positive and negative control) probes and without performing intermediate normalization relying on negative (background-adjusted) or positive (synthetic sequences spiked with known titrations). In embodiments, the T-effector signature score is then calculated as the arithmetic mean of normalized values for each of the genes in the gene signature, e.g., adenosine A2A receptors, CD73, PD-L1.

In embodiments, oligonucleotides in kits are capable of specifically hybridizing to a target region of a polynucleotide, such as for example, an RNA transcript or cDNA generated therefrom. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. The composition and length of each oligonucleotide in the kit will depend on the nature of the transcript containing the target region as well as the type of assay to be performed with the oligonucleotide and is readily determined by the skilled artisan.

### Embodiments

Embodiment 1. A method of treating cancer in a subject in need thereof, the method comprising administering a therapeutically effective amount of an adenosine pathway inhibitor to the subject to treat the cancer; wherein the subject has an elevated level of adenosine A2A receptors when compared to a control.

Embodiment 2. A method of treating cancer in a subject in need thereof, the method comprising administering a therapeutically effective amount of an adenosine pathway inhibitor to the subject to treat the cancer; wherein the subject has: (i) an elevated level of adenosine A2A receptors when compared to a control; and (ii) an elevated level of CD73 when compared to a control.

Embodiment 3. A method of treating cancer in a subject in need thereof, the method comprising administering a therapeutically effective amount of an adenosine pathway inhibitor to the subject to treat the cancer; wherein the subject has: (i) an elevated level of adenosine A2A receptors when compared to a control; (ii) an elevated level of CD73 when compared to a control; and (iii) an elevated level of PD-L1 when compared to a control.

Embodiment 4. A method of treating cancer in a subject in need thereof, the method comprising administering a therapeutically effective amount of an adenosine pathway inhibitor to the subject to treat the cancer; wherein the subject has: (i) an elevated level of adenosine A2A receptors when compared to a control; and (ii) an elevated level of PD-L1 when compared to a control.

Embodiment 5. The method of anyone of Embodiments 1 to 4, wherein the subject has previously been treated with PD-1 pathway inhibitor therapy.

Embodiment 6. The method of Embodiment 5, wherein the PD-1 pathway inhibitor therapy is a PD-L1 inhibitor therapy.

Embodiment 7. The method of Embodiment 5, wherein the PD-1 pathway inhibitor therapy is a PD-1 inhibitor therapy.

Embodiment 8. A method of treating cancer in a subject in need thereof, the method comprising (i) measuring an adenosine A2A receptor level in a biological sample obtained from the subject, and (ii) administering a therapeutically effective amount of an adenosine pathway inhibitor to the subject to treat the cancer.

Embodiment 9. The method of Embodiment 8, further comprising measuring a CD73 level in the biological sample.

Embodiment 10. The method of Embodiment 8 or 9, further comprising measuring a PD-L1 level in a the biological sample.

Embodiment 11. The method of anyone of Embodiments 8 to 10, wherein the biological sample is a tumor sample.

Embodiment 12. The method of Embodiment 11, wherein the tumor sample is a resected tumor sample.

Embodiment 13. The method of Embodiment 11, wherein the tumor sample is a tumor biopsy sample.

Embodiment 14. The method of any one of Embodiments 11 to 13, wherein the tumor sample is from a primary tumor.

Embodiment 15. The method of any one of Embodiments 11 to 13, wherein the tumor sample is from a metastisic tumor.

Embodiment 16. The method of anyone of Embodiments 8 to 10, wherein the biological sample is a blood sample.

Embodiment 17. The method of Embodiment 16, wherein the blood sample is a peripheral blood sample.

Embodiment 18. The method of anyone of Embodiments 1 to 17, wherein the subject is an anti-PD-1 resistant subject.

Embodiment 19. The method of any one of Embodiments 1 to 18, wherein the adenosine pathway inhibitor is an adenosine A2A receptor antagonist, an anti-CD73 compound, an anti-CD39 compound, or a combination of two or more thereof.

Embodiment 20. The method of anyone of Embodiments 1 to 18, wherein the adenosine pathway inhibitor is an adenosine A2A receptor antagonist.

Embodiment 21. The method of anyone of Embodiments 1 to 18, wherein the adenosine pathway inhibitor is a compound of Formula (I) or a pharmaceutically acceptable salt thereof.

Embodiment 22. The method of any one of Embodiments 1 to 18, wherein the adenosine pathway inhibitor is a compound of Formula (II) or a pharmaceutically acceptable salt thereof.

Embodiment 23. The method of any one of Embodiments 1 to 18, wherein the adenosine pathway inhibitor is a compound of Formula (III) or a pharmaceutically acceptable salt thereof.

Embodiment 24. The method of any one of Embodiments 1 to 18, wherein the adenosine pathway inhibitor is a compound of Formula (IIIA) or a pharmaceutically acceptable salt thereof.

Embodiment 25. The method of any one of Embodiments 1 to 18, wherein the adenosine pathway inhibitor is a compound of Formula (IIIB) or a pharmaceutically acceptable salt thereof.

Embodiment 26. The method of any one of Embodiments 1 to 25, wherein the method of treating cancer is a method of increasing CD8-positive cells relative to the amount of regulatory T cells.

Embodiment 27. The method of any one of Embodiments 1 to 25, wherein the method of treating cancer is a method of decreasing tumor volume.

Embodiment 28. The method of any one of Embodiments 1 to 25, wherein the method of treating cancer is a method of enhancing anti-tumor immune memory.

Embodiment 29. The method of any one of Embodiments 1 to 25, wherein the method of treating cancer is a method of treating a cancer tumor.

Embodiment 30. The method of any one of Embodiments 1 to 29, wherein the cancer is lung cancer.

Embodiment 31. The method of Embodiment 30, wherein the lung cancer is non-small cell lung cancer.

Embodiment 32. The method of any one of Embodiments 1 to 29, wherein the cancer is melanoma.

Embodiment 33. The method of Embodiment 32, wherein the melanoma is malignant melanoma.

Embodiment 34. The method of any one of Embodiments 1 to 29, wherein the cancer is breast cancer.

Embodiment 35. The method of Embodiment 34, wherein the breast cancer is triple negative breast cancer.

Embodiment 36. The method of any one of Embodiments 1 to 29, wherein the cancer is colorectal cancer.

Embodiment 37. The method of any one of Embodiments 1 to 29, wherein the cancer is bladder cancer.

Embodiment 38. The method of anyone of Embodiments 1 to 29, wherein the cancer is head and neck cancer.

Embodiment 39. The method of any one of Embodiments 1 to 29, wherein the cancer is renal cell cancer.

Embodiment 40. The method of any one of Embodiments 1 to 29, wherein the cancer is prostate cancer.

Embodiment 41. The method of anyone of Embodiments 1 to 40, further comprising administering a therapeutically effective amount of a chemotherapeutic agent to the subject.

Embodiment 42. A method to identify a subject responsive to an adenosine pathway inhibitor, the method comprising: (i) obtaining a biological sample from the patient; and (ii) measuring an adenosine A2A receptor level in the biological sample; wherein if the adenosine A2A receptor level is elevated when compared to a control, the subject is identified as responsive to the adenosine pathway inhibitor.

Embodiment 43. A method of selecting a subject for treatment with an adenosine pathway inhibitor, the method comprising: (i) obtaining a biological sample from the patient; and (ii) measuring an adenosine A2A receptor level in the biological sample; wherein if the adenosine A2A receptor level is elevated when compared to a control, the subject is selected for treatment with the adenosine pathway inhibitor.

Embodiment 44. The method of Embodiment 42 or 43, further comprising measuring a CD73 level in the biological sample.

Embodiment 45. The method of any one of Embodiments 42 to 44, further comprising measuring a PD-L1 level in the biological sample.

Embodiment 46. The method of any one of Embodiments 42 to 45, wherein the biological sample is a tumor sample.

Embodiment 47. The method of Embodiment 46, wherein the tumor sample is a resected tumor sample.

Embodiment 48. The method of Embodiment 46, wherein the tumor sample is a tumor biopsy sample.

Embodiment 49. The method of any one of Embodiments 46 to 48, wherein the tumor sample is from a primary tumor.

Embodiment 50. The method of any one of Embodiments 46 to 48, wherein the tumor sample is from a metastisic tumor.

Embodiment 51. The method of anyone of Embodiments 42 to 45, wherein the biological sample is a blood sample.

Embodiment 52. The method of Embodiment 51, wherein the blood sample is a peripheral blood sample.

Embodiment 53. The method of anyone of Embodiments 42 to 52, wherein the subject is an anti-PD-1 resistant subject.

Embodiment 54. The method of any one of Embodiments 42 to 53, wherein the adenosine pathway inhibitor is an adenosine A2A receptor antagonist, an anti-CD73 compound, an anti-CD39 compound, or a combination of two or more thereof.

Embodiment 55. The method of any one of Embodiments 42 to 53, wherein the adenosine pathway inhibitor is an adenosine A2A receptor antagonist.

Embodiment 56. The method of any one of Embodiments 42 to 53, wherein the adenosine pathway inhibitor is a compound of Formula (I) or a pharmaceutically acceptable salt thereof.

Embodiment 57. The method of any one of Embodiments 42 to 53, wherein the adenosine pathway inhibitor is a compound of Formula (II) or a pharmaceutically acceptable salt thereof.

Embodiment 58. The method of any one of Embodiments 42 to 53, wherein the adenosine pathway inhibitor is a compound of Formula (III) or a pharmaceutically acceptable salt thereof.

Embodiment 59. The method of any one of Embodiments 42 to 53, wherein the adenosine pathway inhibitor is a compound of Formula (IIIA) or a pharmaceutically acceptable salt thereof.

Embodiment 60. The method of any one of Embodiments 42 to 53, wherein the adenosine pathway inhibitor is a compound of Formula (IIIB) or a pharmaceutically acceptable salt thereof.

Embodiment 61. The method of anyone of Embodiments 42 to 60, wherein the subject has cancer.

Embodiment 62. The method of Embodiment 61, wherein the cancer is lung cancer, melanoma, breast cancer, colorectal cancer, renal cancer, bladder cancer, a head and neck cancer, or prostate cancer.

Embodiment 63. The method of Embodiment 61, wherein the cancer is non-small cell lung cancer, malignant melanoma, or triple negative breast cancer.

Embodiment 64. The method of any one of Embodiments 61 to 63, further comprising administering a therapeutically effective amount of an adenosine pathway inhibitor to the subject to treat the cancer.

Embodiment 65. The method of Embodiment 64, wherein the adenosine pathway inhibitor is an adenosine A2A receptor antagonist, an anti-CD73 compound, an anti-CD39 compound, or a combination of two or more thereof.

Embodiment 66. The method of Embodiment 64, wherein the adenosine pathway inhibitor is an adenosine A2A receptor antagonist.

Embodiment 67. The method of Embodiment 64, wherein the adenosine pathway inhibitor is a compound of Formula (I) or a pharmaceutically acceptable salt thereof.

Embodiment 68. The method of Embodiment 64, wherein the adenosine pathway inhibitor is a compound of Formula (II) or a pharmaceutically acceptable salt thereof.

Embodiment 69. The method of Embodiment 64, wherein the adenosine pathway inhibitor is a compound of Formula (III) or a pharmaceutically acceptable salt thereof.

Embodiment 70. The method of Embodiment 64, wherein the adenosine pathway inhibitor is a compound of Formula (IIIA) or a pharmaceutically acceptable salt thereof.

Embodiment 71. The method of Embodiment 64, wherein the adenosine pathway inhibitor is a compound of Formula (IIIB) or a pharmaceutically acceptable salt thereof.

Embodiment 72. The method of anyone of Embodiments 64 to 71, further comprising administering a therapeutically effective amount of a chemotherapeutic agent to the subject.

Embodiment 73. A method of determining whether a cancer patient expresses high adenosine A2A receptor levels, the method comprising: (i) obtaining a biological sample from the patient; and (ii) measuring the adenosine A2A receptor levels in the biological sample.

Embodiment 74. A method of determining whether a cancer patient expresses high adenosine A2A receptor levels and high CD73 levels, the method comprising: (i) obtaining a biological sample from the patient; and (ii) measuring the adenosine A2A receptor levels and the CD73 levels in the biological sample.

Embodiment 75. The method of Embodiment 73 or 74, further comprising measuring PD-L1 levels in the biological sample to determine whether the cancer patient expresses high PD-L1 levels.

Embodiment 76. The method of any one of Embodiments 73 to 75, wherein the biological sample is a tumor sample.

Embodiment 77. The method of Embodiment 76, wherein the tumor sample is a resected tumor sample.

Embodiment 78. The method of Embodiment 76, wherein the tumor sample is a tumor biopsy sample.

Embodiment 79. The method of any one of Embodiments 76 to 78, wherein the tumor sample is from a primary tumor.

Embodiment 80. The method of any one of Embodiments 76 to 78, wherein the tumor sample is from a metastisic tumor.

Embodiment 81. The method of anyone of Embodiments 73 to 75, wherein the biological sample is a blood sample.

Embodiment 82. The method of Embodiment 81, wherein the blood sample is a peripheral blood sample.

### Examples

The following examples are for purposes of illustration only and are not intended to limit the scope of the disclosure or claims.

The compound of Formula (III), also known as CPI-444, inhibits suppression of T cell function by adenosine and is active in multiple preclinical models. The compound of Formula (III) is being investigated in an ongoing Phase 1/1b clinical trial in patients with advanced cancers (NCT02655822 at www.clinicaltrials.gov). Biomarker investigations were conducted to explore immune modulation in serial tumor biopsies and peripheral blood as well associations between adenosine pathway genes and clinical activity.

Cancer patients having renal cell cancer, lung cancer (e.g., non-small cell lung cancer), melanoma, breast cancer (e.g., triple negative breast cancer), prostate cancer, bladder cancer, colorectal cancer, and other types of cancer entered into clinical trials. Cancer patients were permitted to enter the trial if they were anti-PD-1 resistant or anti-PD-1 refractory. Prior to treatment, the patients' expression levels of adenosine A2A receptors, CD73, and CD39 were analyzed and the results are shown in FIG. 1A, FIG. 1B, and FIG. 1C, respectively. Similarly, the patients' expression levels of adenosine A2A receptors, CD73, and CD39 were analyzed and are shown in FIG. 2A, FIG. 2B, and FIG. 2C, respectively, based on patients with renal cell cancer, non-small cell lung cancer, and other cancers. Tumor expression of adenosine A2A receptors, CD73, and CD39 are increased in patients that are resistant to prior treatment with PD-L1 inhibitors. Renal cell cancer and non-small cell lung cancer have high tumor expression of adenosine A2A receptors genes, CD73, and CD79.

The patients were orally administered the compound of Formula (III) at a dose of 100 mg, twice a day, for twenty-eight days. Tumor biopsies and blood samples were analyzed from patients with renal cell carcinoma, non-small cell lung cancer, triple negative breast cancer, microsatellite instable colorectal cancer treated with the compound of Formula (III). Paired tumor biopsies were analyzed: gene expression profiles (Nanostring), CD8, PD-L1 and CD73 (IHC). T cell repertoires were examined by sequencing of the T cell receptor beta chain gene in PBMCs and tumors.

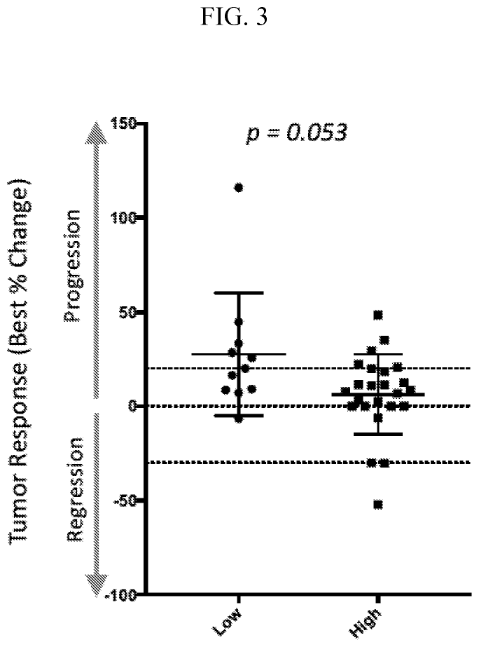

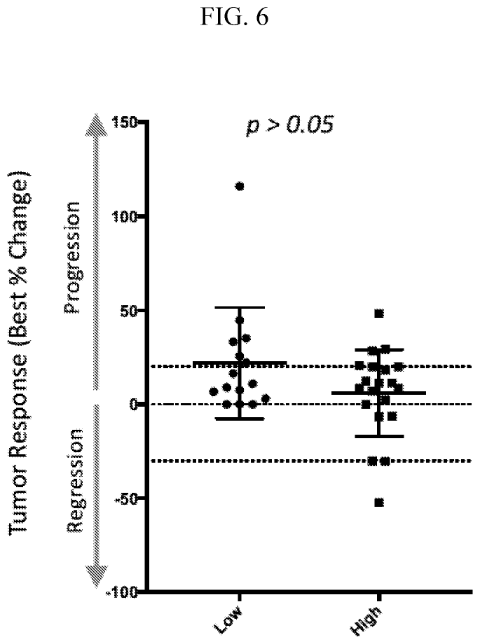

With reference to FIGS. 3-6, the range of A2AR gene expression, CD73 gene expression, PD-L1 gene expression, and PD-L1 staining on immune cells was determined for all screened subjects, and cut-offs were established as follows: (i) ≥the first quartile of CD73 was high for CD73; (ii) ≥the median of adenosine A2A receptors was high for adenosine A2A receptors; and (iii) the 4^{th} quartile of PD-L1 was high for PD-L1 high. The best observed percent change in the sum of longest dimensions of their assessed target tumor lesions was plotted.

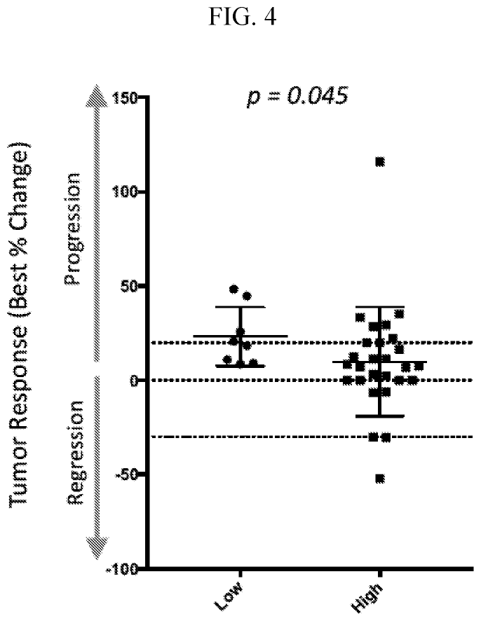

FIG. 3 and FIG. 4 show that patients having elevated expression levels of adenosine A2A receptors and CD73 had better treatment results than patients who had low expression levels of adenosine A2A receptors and CD73.

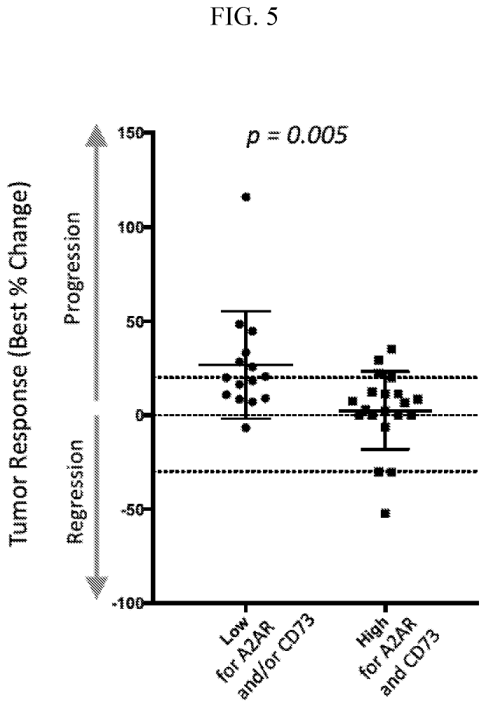

FIG. 5 shows the results from the treatment of patients with double positive adenosine A2A receptors and CD73 (i.e., high CD73 and high adenosine A2A receptors) when compared to patients with low expression of adenosine A2A receptors and/or CD73.

As shown in FIGS. 3-7, patients with elevated expression of adenosine A2A receptor genes, CD73, and PD-L1 in baseline tumor samples experienced significant tumor regression from treatment with the compound of Formula (III) when compared to patients having low expression of adenosine A2A receptor genes, CD73, and PD-L1 in baseline tumor samples

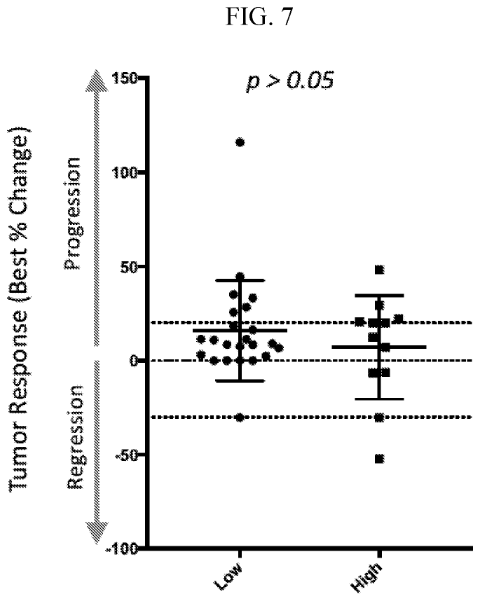

For PD-L1 IHC, immune cell staining ≥1% of tumor are was considered high and <1% is low, as shown in FIG. 7.

The anti-tumor activity of the treatment was associated with immune-modulation of T cells in tumor and periphery, including IO-refractory/IO-resistant and PD-L1 negative patients. Adenosine pathway is upregulated in IO-refractory/IO-resistant patients, and was associated with clinical response to the treatment of IO-refractory/IO-resistant tumors.

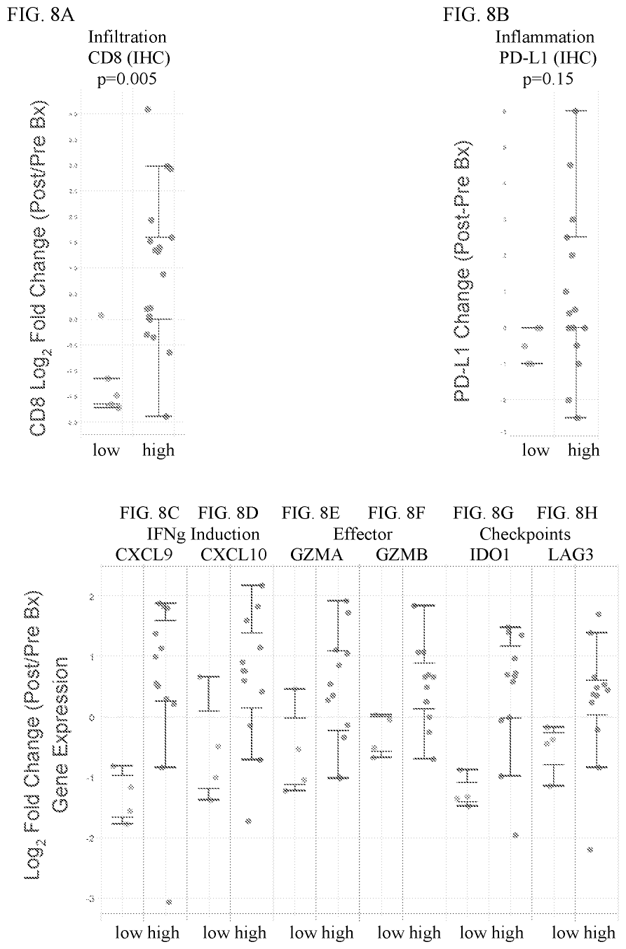

FIGS. 8A-8H show the changes in immunohistochemistry and gene expression by Nanostring on on-treatment biopsies compared to pre-treatment biopsies and identifies pharmacodynamics changes that are observed only in CD73-high tumors treated with the compound of Formula (III).

While various embodiments and aspects of the disclosure are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments described herein may be employed.

1-55. (canceled)

56. A method of treating cancer in a subject in need thereof, the method comprising administering a therapeutically effective amount of an adenosine pathway inhibitor to the subject to treat the cancer; wherein the subject has an elevated level of adenosine A2A receptors when compared to a control; and wherein the subject optionally has (i) an elevated level of CD73 when compared to a control; (ii) an elevated level of PD-L1 when compared to a control; or (iii) an elevated level of CD73 when compared to a control and an elevated level of PD-L1 when compared to a control.

57. The method of claim 56, wherein the subject has previously been treated with PD-1 pathway inhibitor therapy.

58. The method of claim 56, further comprising measuring an adenosine A2A receptor level in a biological sample obtained from the subject, measuring a CD73 level in a biological sample, measuring a PD-L1 level in a biological sample, or a combination of two or more thereof.

59. The method of claim 58, wherein the biological sample is a tumor sample.

60. The method of claim 59, wherein the tumor sample is a resected tumor sample or a tumor biopsy sample.

61. The method of claim 58, wherein the biological sample is a blood sample.

62. The method of claim 61, wherein the blood sample is a peripheral blood sample.

63. The method of claim 56, wherein the subject is an anti-PD-1 resistant subject.

64. The method of claim 56, wherein the adenosine pathway inhibitor is an adenosine A2A receptor antagonist, an anti-CD73 compound, an anti-CD39 compound, or a combination of two or more thereof.

65. The method of claim 64, wherein the adenosine pathway inhibitor is an adenosine A2A receptor antagonist.

66. The method of claim 56, wherein the adenosine pathway inhibitor is a compound of Formula (II) or a pharmaceutically acceptable salt thereof: 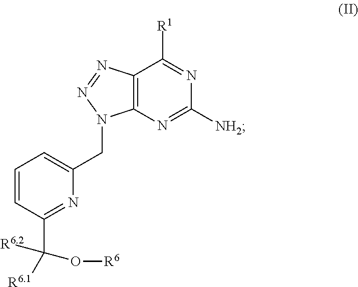
wherein R¹ is independently hydrogen, halogen, —CX^{a}₃, —CN, —SO₂Cl, —SOₙ₁R⁹, —SOᵥ₁NR⁹R¹⁰, —NHNH₂, —ONR⁹R¹⁰, —NHC═(O)NHNH₂, —NHC═(O)NR⁹R¹⁰, —N(O)ₘ₁, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶, R^{6.1} and R^{6.2} are independently hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ and R¹⁰ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X^{a} is —F, —Cl, —Br, or —I; n₁ is an integer from 0 to 4; m₁ is 1 or 2; and v₁ is 1 or 2.

67. The method of claim 56, wherein the adenosine pathway inhibitor is a compound of Formula (III) or a pharmaceutically acceptable salt thereof:

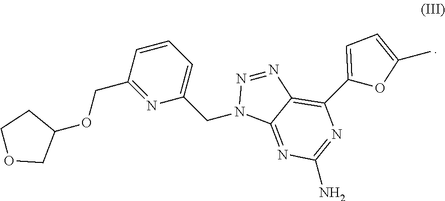 [SMILES: Cc1ccc(-c2nc(N)nc3c2nnn3Cc2cccc(COC3CCOC3)n2)o1]

68. The method of claim 56, wherein the method of treating cancer is a method of increasing CD8-positive cells relative to the amount of regulatory T cells or a method of enhancing anti-tumor immune memory.

69. The method of claim 56, wherein the method of treating cancer is a method of decreasing tumor volume.

70. The method of claim 56, wherein the cancer is lung cancer, melanoma, breast cancer, colorectal cancer, bladder cancer, head and neck cancer, renal cell cancer, or prostate cancer.

71. The method of claim 56, wherein the cancer is renal cell cancer.

72. The method of claim 56, further comprising administering a therapeutically effective amount of a chemotherapeutic agent to the subject.

73. A method to treat cancer in a subject responsive to an adenosine pathway inhibitor, the method comprising:
(i) obtaining a biological sample from the patient;
(ii) measuring an adenosine A2A receptor level in the biological sample; wherein if the adenosine A2A receptor level is elevated when compared to a control, the subject is identified as responsive to the adenosine pathway inhibitor; and
(iii) administering to the subject an effective amount of the adenosine pathway inhibitor.

74. The method of claim 73, wherein the cancer is lung cancer, melanoma, breast cancer, colorectal cancer, renal cancer, bladder cancer, a head and neck cancer, or prostate cancer.

75. The method of claim 73, wherein the adenosine pathway inhibitor is a compound of Formula (III) or a pharmaceutically acceptable salt thereof, and wherein the PD-1 pathway inhibitor is atezolizumab; wherein the compound of Formula (III) is: 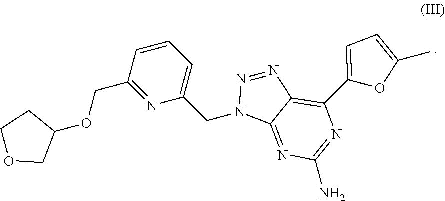 [SMILES: Cc1ccc(-c2nc(N)nc3c2nnn3Cc2cccc(COC3CCOC3)n2)o1]
